(19) United States
(12) Patent Application Publication
(10) Pub. No.: US 2021/0228144 A1
(43) Pub. Date: Jul. 29, 2021

(54) SYSTEM, METHOD AND COMPUTER-ACCESSIBLE MEDIUM FOR NEUROMELANIN-SENSITIVE MAGNETIC RESONANCE IMAGING AS A NON-INVASIVE PROXY MEASURE OF DOPAMINE FUNCTION IN THE HUMAN BRAIN

(71) Applicants: The Trustees of Columbia University in the City of New York, New York, NY (US); THE RESEARCH FOUNDATION FOR MENTAL HYGIENE, INC., MENANDS, NY (US)

(72) Inventors: GUILLERMO HORGA HERNANDEZ, Brooklyn, NY (US); CLIFFORD MILLS CASSIDY, Ottawa (CA)

(73) Assignees: The Trustees of Columbia University in the City of New York; THE RESEARCH FOUNDATION FOR MENTAL HYGIENE, INC.

(21) Appl. No.: 17/226,375

(22) Filed: Apr. 9, 2021

Related U.S. Application Data
(63) Continuation of application No. PCT/US2019/055652, filed on Oct. 10, 2019.
(60) Provisional application No. 62/743,916, filed on Oct. 10, 2018.

(51) Int. Cl. A61B 5/00; A61B 5/055; G06T 7/00; G06T 7/60; G01R 33/48; G01R 33/483

(52) U.S. Cl. CPC A61B 5/4082; A61B 5/055; A61B 5/0042; G06T 7/0014; G06T 2207/30016; G01R 33/4806; G01R 33/4835; G06T 2207/10088; G06T 7/60

(57) ABSTRACT

An exemplary system, method and computer-accessible medium for determining a dopamine function of a patient(s) can include, for example, receiving imaging information of a brain of the patient(s), determining a Neuromelanin (NM) concentration of the patient(s) based on the imaging information, and determining the dopamine function based on the NM concentration. The NM concentration can be determined using a voxel-wise analysis procedure. The voxel-wise analysis procedure can be used to determine a topographical pattern(s) within a substantia nigra (SN) of the brain of the patient(s). The topographical pattern(s) can include a pattern(s) of cell loss in the SN. The NM concentration can be based on a NM loss in the brain of the patient(s).

### CROSS-REFERENCE TO RELATED APPLICATION(S)

This application relates to and claims priority from U.S. Patent Application No. 62/743,916, filed on Oct. 10, 2018, the entire disclosure of which is incorporated herein by reference.

### STATEMENT REGARDING FEDERALLY SPONSORED RESEARCH

This invention was made with government support under Grant No. R01MH17323, awarded by the National Institute of Mental Health. The government has certain rights in the invention

### FIELD OF THE DISCLOSURE

The present disclosure relates generally to magnetic resonance imaging (“MRI”), and more specifically, to exemplary embodiments of an exemplary system, method and computer-accessible medium for neuromelanin-sensitive MRI as a non-invasive proxy measure of dopamine function in the human brain.

### BACKGROUND INFORMATION

Its vivo measurements of dopamine activity are used for understanding how neuromodulator contributes to cognition, neurodevelopment, aging, and neuropsychiatric disease in humans. In medicine, such measurements can result in objective biomarkers that predict clinical outcomes in dopamine-related illness, including Parkinson's disease (“PD”) and psychotic disorders, ideally by using procedures that capture the underlying pathophysiology while being easy to acquire in clinical settings. (See, e.g., Reference 1).

Neuromelanin (“NM”) is a dark pigment synthesized via iron-dependent oxidation of cytosolic dopamine and subsequent relation with proteins and lipids in midbrain dopamine neurons. (See, e.g., Reference 2). NM pigment accumulates inside specific autophagic organelles, which contain. NM-iron complexes, along with lipids and various proteins. (See, Reference 3). NM-containing organelles accumulate gradually over the lifespan in the soma of dopamine neurons in the substantia nigra (“SN”) (see, e.g., Reference 4), a nucleus that owes its name to its dark appearance due to the high concentration of NM, and are only cleared from tissue following cell death through the action of microglia, as in neurodegenerative conditions such as PD. (See, e.g., References 5 and 6). Given that NM-iron complexes are paramagnetic (see, e.g. References 6 and 7), they can be imaged using MRI. (See, e.g., References 8-11).

A family of MRI sequences known as NM-MRI captures groups of neurons with high NM content, such as those in the SN, as hyperintense regions. (See, e.g., References 8 and 9). NM-MRI signal is reliably decreased in the SN of patients with PD (see, e.g., References 8 and 10, and 12-15), consistent with the degeneration of NM-positive SN dopamine cells (see, e.g., Reference 16) and with the decrease in NM concentration in post mortem SN tissue of PD patients compared to age-matched controls. (See, e.g., Reference 17). While this evidence supports the use of NM-MRI for in vivo detection of SN neuron loss in neurodegenerative illness, direct demonstrations that this MRI procedure is sensitive to regional variability in NM concentration even in the absence of neurodegenerative SN pathology are lacking. Furthermore, although induction of dopamine synthesis via L-DOPA administration is known to induce NM accumulation in rodent SN cells (see, e.g., References 18 and 19), and although prior work assumed that NM-MR signal in the SN indexes dopamine neuron function in humans (see, e.g., References 20 and 21), direct evidence is lacking to support the assumption that inter-individual differences in dopamine function could lead to MRI-detectable differences in NM accumulation. Such evidence is needed to support the utility of NM-MRI for psychiatric and neuroscientific applications beyond those related to neurodegenerative illness.

The paramagnetic nature of the NM-iron complexes within the NM-containing organelles, (see, e.g., References 156 and 157), facilitates them to be nonvasively imaged using MRI. (See, e.g., References 97, 135, 143 and 146). NM-MRI produces hyperintense signals in neuromelanin-containing regions such as the SN and LC due to the short longitudinal relaxation time (T₁) of the NM-complexes and saturation attic surrounding white matter “WM”) by either direct magnetization transfer (“MT”) pulses, (see. e.g., Reference 99) or indirect MT effects, (See, e.g., References 135 and 146). While many previous NM-MRI studies have used indirect MT effects, images with direct MT pulses achieve greater sensitivity (see, e.g., References 120 and 138), and were recently shown to be directly related to NM concentration. (See, e.g., Reference 8).

NM-MRI has also been validated as a measure of dopaminergic neuron loss in the SN (see, e.g., Reference 118), and several studies have shown that this method can capture the well-known loss of NM-containing neurons in the SN of individuals with Parkinson's disease. (See, e.g., Reference 143). More recently, NM-MRI was validated as a marker eat dopamine function, with the NM-MRI signal in the SN demonstrating a significant relationship to PET measures of dopamine release capacity in the striatum. (See, e.g., Reference 97). Furthermore, a voxelwise-analysis approach was validated to resolve substructures within dopaminergic nuclei thought to have distinct anatomical targets and functional roles. (See, e.g., References 97, 110, 133 and 151), This voxel-wise approach can thus facilitate a more anatomically precise interrogation of specific midbrain circuits encompassing subregions within the SN or small nuclei such as the ventral tegmental area (“VTA”), which can in turn increase the accuracy of NM-MRI markers for clinical or mechanistic research. For example, voxel-wise NM-MRI can facilitate investigations into the specific subregions within the SN/VTA-complex projecting to the head of the caudate, which can be of particular relevance in the study of psychosis, (see, e.g., Reference 151), or assist in capturing the known topography of SN neuronal loss in Parkinson's disease. (See, e.g., References 97, 102 and 107).

An additional benefit of a voxelwise-analysis can include avoiding the circularity that can incur when defining ROIs based on the NM-MRI images that can then be used to read out the signal in those same regions. Most previous studies have used the high signal region in the NM-MRI images to define the SN ROI that can he used for further analysis. While this can be appropriate if the goal of the study can he to measure the volume of the SN, it can be problematic for analysis of the CNR because the selected ROI can be biased towards high CNR voxels.

NM-MRI can be used to nonitivasively interrogate in vivo the dopamine system. However, this can he dependent on a thorough investigation of the performance of the method for various acquisition parameters and preprocessing methods. In particular, most previous studies used relatively thick MRI slices (e.g, approximately 3 mm) (see, e.g., References 121, 134 and 139), compared to the in-plane resolution (approximately 0.5 mm) and to the height of the SN (approximately 15 mm) (see, e.g., Reference 130), to overcome technical limitations such as specific absorption rate and SNR, at the cost of partial-volume effects. Additionally, previous studies acquired multiple measurements that were subsequently averaged to improve the SNR of the exemplary procedure with the expense of increased scanning time. While time in the MRI can be expensive and should be minimized to improve patient compliance, a detailed investigation into how many measurements can be needed for robust NM-MRI has not been reported. A recent study indicated high reproducibility for ROI-analysis. (See, e.g., Reference 121). However, this study did not provide a detailed description of NM-MRI volume placement and the two scans were obtained within a single session although subjects were removed and repositioned in-between scans.

Thus, it may be beneficial to provide an exemplary system, method and computer-accessible medium for neuromelanin-sensitive MRI as a non-invasive proxy measure of dopamine function in the human brain which can overcome at least some of the deficiencies described herein above.

### SUMMARY OF EXEMPLARY EMBODIMENTS

An exemplary system, method and computer-accessible medium for determining a dopamine function of a patient(s) can include, for example, receiving imaging information of a brain of the patient(s), determining a Neuromelanin (NM) concentration of the patient(s) based on the imaging information, and determining the dopamine function based on the NM concentration. The NM concentration can be determined using a voxel-wise analysis procedure. The voxel-wise analysis procedure can be used to determine a topographical pattern(s) within a substantia nigra (SN) of the brain of the patient(s). The topographical pattern(s) can include a pattern(s) of cell loss in the SN. The NM concentration can be based on a NM loss in the brain of the patient(s).

In some exemplary embodiments of the present disclosure, the imaging information can be magnetic resonance imaging (“MRI”) information. A variance in the NM concentration can be determined using a NM-MRI contrast-to-noise ratio (“CNR”). The NM-MRI CNR can be determined at each voxel in the imaging information. The NM-MRI CNR can be determined as a relative change in a NM-MRI signal intensity from a reference region of white matter tracts in the brain of the patient(s). Information correlating with a brain disorder of the patient(s) can be determined based on the dopamine function. The brain disorder can include (i) schizophrenia, (ii) bipolar disorder, or (iii) Parkinson's disease. Further information correlating with a severity of the brain disorder can be determined based on the dopamine function.

In certain exemplary embodiments of the present disclosure, the imaging information can include (i) sagittal three-dimensional (3D) T1w image(s), (ii) a coronal 3D T1w image(s), and (iii) a axial 3D T1w image(s). A magnetic resonance imaging (“MRI”) volume placement can be determined in the imaging information by, for example, (i) identifying a sagittal image showing a largest separation between a midbrain of the patient(s) and a thalamus of the patient(s), (ii) determining a coronal image that has a coronal plane in the sagittal image that identifies a most anterior aspect of the midbrain, (iii) determining an axial plane in the coronal image that identifies an inferior aspect of a third ventricle of the brain of the patient(s), and setting a superior boundary of the NM-MRI volume to be within a particular distance superior to the axial plane. The particular distance can be about 3 mm.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A and 1C are exemplary images of an axial view of a post-mortem specimen of the right hemi-midbrain according to an exemplary embodiment of the present disclosure;

FIGS. 1B and 1D are exemplary NM-MRI images according to an exemplary embodiment of the present disclosure;

FIG. 1E is an exemplary Scatterplot displaying the correlation between NM concentration and NM-MRI contrast-to-noise ratio (“CNR”) for a single specimen according to an exemplary embodiment of the present disclosure;

FIG. 1F is an exemplary scatterplot displaying the correlation between NM concentration and NM-MRI CNR for 7 specimens according to an exemplary embodiment of the present disclosure;

FIG. 2A is an exemplary Template NM-MRI image created by averaging the spatially normalized NM-MRI images according to an exemplary embodiment of the present disclosure:

FIG. 2B is an exemplary image of masks for the substantia nigra and the crus cerebri reference region according to an exemplary embodiment of the present disclosure;

FIG. 2C is a set of exemplary three-dimensional (“3D”) images and signal change diagrams according to an exemplary embodiment of the present disclosure;

FIG. 3A is an exemplary set of raw NM-MRI images of the midbrain according to an exemplary embodiment of the present disclosure;

FIG. 3B is an exemplary image and T-statistic maps of the SN showing the size of the signal decrease in NM-MRI CNR in PD compared to matched controls according to an exemplary embodiment of the present disclosure;

FIG. 4A is an exemplary image and graph of SN voxels where NM-MRI CNR positively correlated with a Positron Emission Tomography (“PET”) measure of dopamine release capacity in the associative striatum overlaid on the NM-MRI template image according to an exemplary embodiment of the present disclosure;

FIG. 4B is an exemplary map and graph of a mean resting cerebral blood flow (“BF”) according to an exemplary embodiment of the present disclosure;

FIG. 5 is an exemplary image and a set of graphs showing how NM-MRI CNR correlates with the severity of psychotic symptoms according to an exemplary embodiment of the present disclosure;

FIG. 6 is a set of exemplary images of a quality check attic spatial normalization procedures according to an exemplary embodiment of the present disclosure;

FIG. 7A is an exemplary map of intraclass correlation coefficient values (“ICC”) across voxels in the SN according to an exemplary embodiment of the present disclosure;

FIG. 7B is an exemplary scatterplot showing agreement in NM-MRI CNR for all voxels and all subjects between two scans according to an exemplary embodiment of the present disclosure;

FIG. 8 is an exemplary map and graph illustrating a comparison of PD patients and matched controls according to an exemplary embodiment of the present disclosure;

FIGS. 9A and 9B are exemplary scatterplots illustrating how NM-MRI CNR correlates with measures of dopamine function across individuals without neurodegenerative illness according to an exemplary embodiment of the present disclosure;

FIG. 10A is an exemplary graph illustrating a comparison of clinical high-risk individuals for psychosis to age-matched healthy controls according to an exemplary embodiment of the present disclosure;

FIG. 10B is an exemplary graph illustrating a comparison of unmedicated patients with schizophrenia to age-matched healthy controls according to an exemplary embodiment of the present disclosure;

FIGS. 11A-11E are exemplary images generated using the exemplary system, method and computer-accessible medium according to an exemplary embodiment of the present disclosure;

FIG. 12 is a set of exemplary images of the final MI-MRI volume placement from a representative subject according to an exemplary embodiment of the present disclosure;

FIGS. 13A-13D are exemplary images showing the ROIs overlaid on a template NM image according to an exemplary embodiment of the present disclosure;

FIGS. 14A-14D are exemplary graphs illustrating ICC_{ROI} and CNR_{ROI} within the manually traced mask as a function of acquisition time for each of the NM-MRI sequences and spatial normalization software according to an exemplary embodiment of the present disclosure;

FIGS. 15A-15D are exemplary graphs illustrating the ICC_{ASV}, ICC_{WSV}, and CNR_{V} within the manually traced is as a function of acquisition time for each of the NM-MRI sequences and spatial normalization software according to an exemplary embodiment of the present disclosure; p FIGS. 16A-16D are exemplary scatterplots of the ICC_{ASV} and CNR_{V} of each voxel within the manually traced mask for each of the NM-MRI sequences and spatial normalization software are shown in the scatter plots according to an exemplary embodiment of the present disclosure;

FIG. 17A is an exemplary graph of the predictive value (R²) of anatomical position ICC_{ASV} and ICC_{ASV} and ICC_{ASV} of voxels within the manually traced mask of the SN/VTA-complex (see e.g., FIG. 13B) for NM-1.5 mm sequence and each of the spatial normalization software according to an exemplary embodiment of the present disclosure;

FIG. 17B is an exemplary histogram of ICC_{ASV} of voxels within the manually traced mask for NM-1.5 mm sequence and ANTS spatial normalization software according to an exemplary embodiment of the present disclosure;

FIG. 17C is an exemplary histogram of ICC_{ASV} of voxels within the manually traced mask for NM-1.5 mm sequence and SPM12 spatial normalization software which can be the worst performing method as shown in FIG. 17A according to an exemplary embodiment of the present disclosure;

FIG. 18 is an exemplary graph illustrating the effect of spatial smoothing on ICC_{ASV} and CNR_{V} of voxels within the manually traced mask of the SN/ATA-complex according to an exemplary embodiment of the present disclosure;

FIGS. 19A-19D are exemplary graphs illustrating ICC_{ROI} and CNR_{ROI} within the probabilistic masks as a function of acquisition tune for the NM-1.5 mm sequence and ANTs spatial normalization software and various probability cutoffs according to an exemplary embodiment of the present disclosure;

FIG. 20 is a set of exemplary graphs of correlations and histograms of the CNR_{ROI} values within the 4 SN/VTA7-complex nuclei according to an exemplary embodiment of the present disclosure;

FIG. 21 is a flow diagram of an exemplary method for determining a dopamine function of a patient according to an exemplary embodiment of the present disclosure; and

FIG. 22 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

To illustrate extending the use of NM-MRI for such applications, a series of validation studies is shown. A first procedure is provided to show that NM-MRI can be sensitive enough to detect regional variability in tissue concentration of NM, which can depend on inter-individual and inter-regional differences in dopamine function (e.g., including synthesis and storage capacity), and not just due to loss of NM-containing neurons due to neurodegeneration. MRI measurements were compared to neurochemical measurements of NM concentration in post-mortem tissue without neurodegenerative SN pathology. Because variability in dopamine function may not occur uniformly throughout all SN tiers (see, e.g., References 22-26), the next procedure was to show that NM-MRI, which has high anatomical resolution compared to standard molecular-imaging procedures, has sufficient anatomical specificity. NM-MRI was used as a marker of degeneration in PD to test the ability of an exemplary voxelwise approach to capture the known topographical pattern of cell loss within the SN in the illness. (See, e.g., References 27 and 28). The next exemplary procedure was then to provide direct evidence for a relationship between NM-MRI and dopamine function using the voxelwise approach.

The NM-MRI signal was correlated to a well-validated PET measure of dopamine release into the striatum the main projection site of SN neurons and to a functional MRI measure of regional blood flow in the SN, an indirect measure of activity in SN neurons, in a group of individuals without neurodegenerative illness. NM-MRI was also tested for non-neurodegenerative psychiatric illness (e.g., illness without known neurodegeneration at the cellular level (see, e.g., References 24 and 29)): this procedure was used in unmedicated patients with schizophrenia and individuals at clinical high risk (“CHR”) for psychosis to test the ability of NM-MRI to capture a psychosis-related functional phenotype consisting of nigrostriatal dopamine excess.

### Exemplary Validation of NM-MRI as a Proxy Measure of Dopamine Function

### Exemplary Relationship to Nm Concentration in Post-Mortem Midbrain Tissue

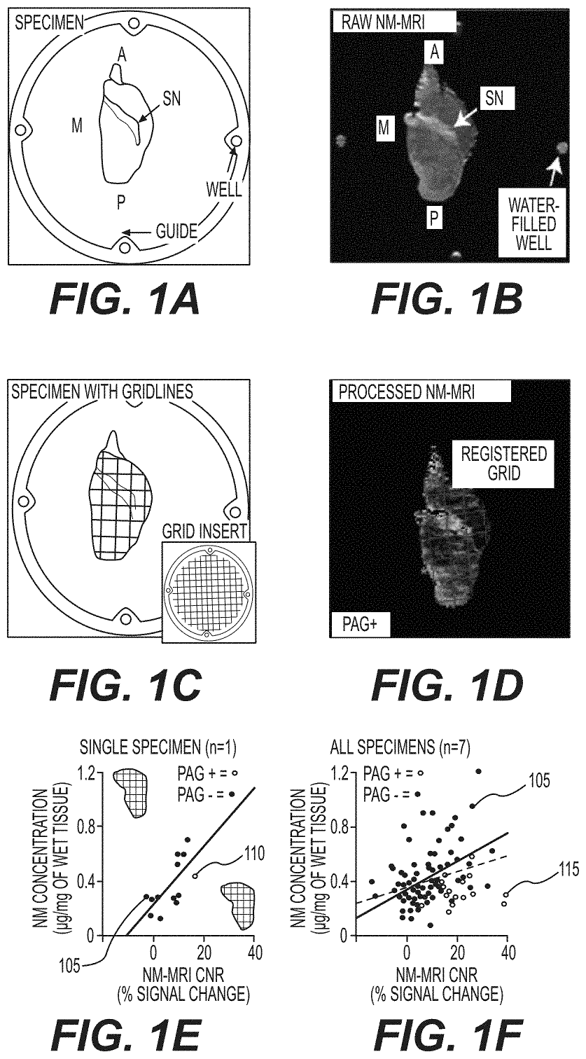

FIGS. 1A and 1C show exemplary images of an axial view of a post-mortem specimen of the right hemi-midbrain according to an exemplary embodiment of the present disclosure. FIGS. 1B and 1D show exemplary NM-MRI images according to an exemplary embodiment of the present disclosure. FIG. 1E shows an exemplary Scatterplot displaying the correlation between NM concentration and NM-MRI CNR for a single specimen according to an exemplary embodiment of the present disclosure. FIG. 1F shows an exemplary scatterplot displaying the correlation between NM concentration and NM-MRI CNR for 7 specimens according to an exemplary embodiment of the present disclosure;

A test was performed to determine whether NM-MRI can be sensitive to variation in NM tissue concentration at levels found in individuals without major neurodegeneration of the SN, a prerequisite tier its use as a marker of inter-individual variability in dopamine function in healthy and psychiatric populations. To this end, this was validated against gold-standard measures of NM concentration by scanning SN-containing midbrain sections from 7 individuals without histopathology compatible with PD or PD-related syndromes (e.g., including absence of Lewy bodies composed of abnormal protein aggregates) using an exemplary NM-MRI sequence. After scanning, each specimen was dissected along gridline markings into 13-20 grid sections. In each grid section, the tissue concentration of was measured using biochemical separation and spectrophotometry determination and also calculated the averaged NM-MRI CNR across voxels within the grid section. (See, e.g., images shown in FIGS. 1A-1D).

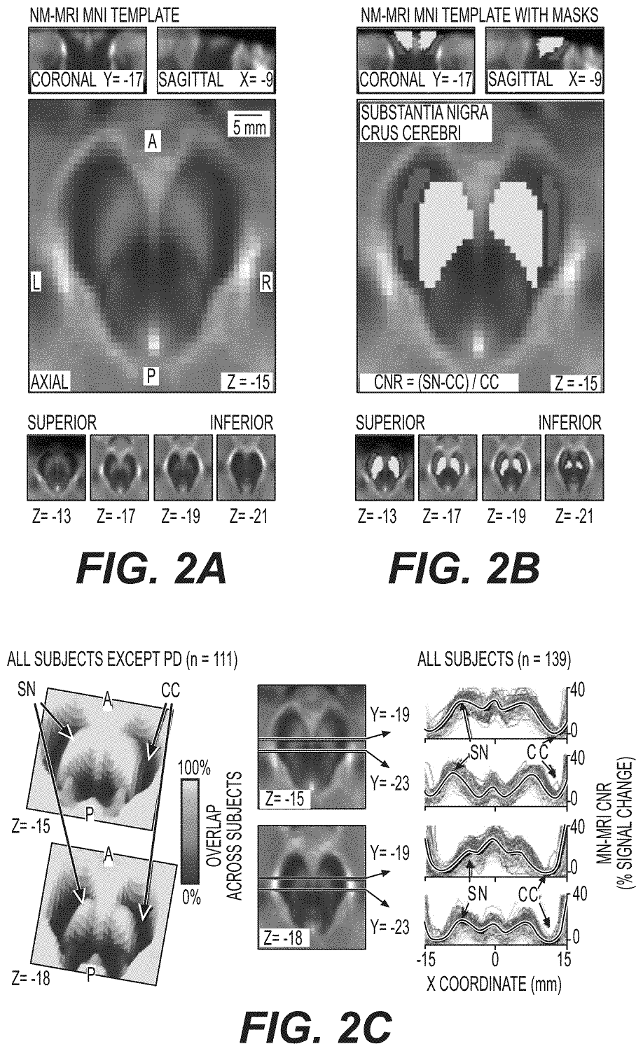

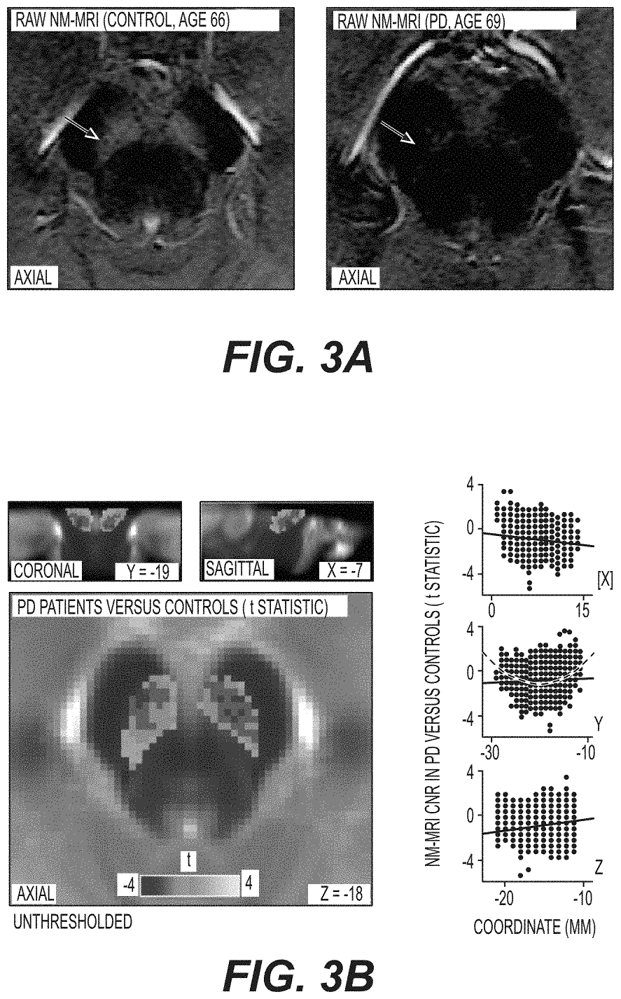

Across all midbrain specimens, grid sections with higher NM-MRI CNR had higher tissue concentration of NM (β₁=0.56, t₁₁₄=3.36, p=0.001, mixed-effects model; 116 grid sections, 7 specimens. (See, e.g., graphs shown in FIGS. 1F-1F). Hyperintensities were most apparent in grid sections corresponding with the NM-rich SN. But, similar to in vivo NM-MRI images (see, e.g., FIGS. 2A-2C, 3A, and 3B), posterior-medial regions of the midbrain around the periaqueductal gray (“PAG”) area tended to appear hyperintense despite relatively low concentrations of NM. Although the source of this hyperintensity, controlling for the presence of PAG (e.g., PAG− shown as element 105 and PAG+ shown as element 110) in grid sections improved the correspondence of NM-MRI CNR to NM concentration, in non-PAG regions (β₁=1.03, t₁₁₂=5.51, p=10⁻⁷) could not he accounted for. Under this model, a 10% increase in NM-MRI CNR corresponds with an estimated increase of 0.10 μg of NM per mg of tissue.

The relationship between NM-MRI CNR and NM concentration remained (β=0.45, t₁₁₁=2.15, p=0.034) in an extended model controlling for the proportion of SN voxels within each grid section (e.g, and again for PIG content). This latter result indicates that NM-MRI CNR can explain the variance in NM concentration in the SN and surrounding areas beyond that explained simply by the increase in both measures in SN compared to non-SN voxels, an increase that can be expected even if NM-MRI could only localize the SN without measuring regional NM concentration. These results thus indicate that NM-MRI signal corresponds to regional tissue concentration of NM, particularly in the midbrain region surrounding the SN, the region focused on. All results held after excluding one specimen in which the neuropathological exam found decreased neuronal density in the SN despite lack of evidence for PD-related pathology (e.g., extended model: β=0.46, t₉₆=2.20, p=0.030), further confirming that the relationship between NM-MRI and NM concentration was not driven by reduced cell counts.

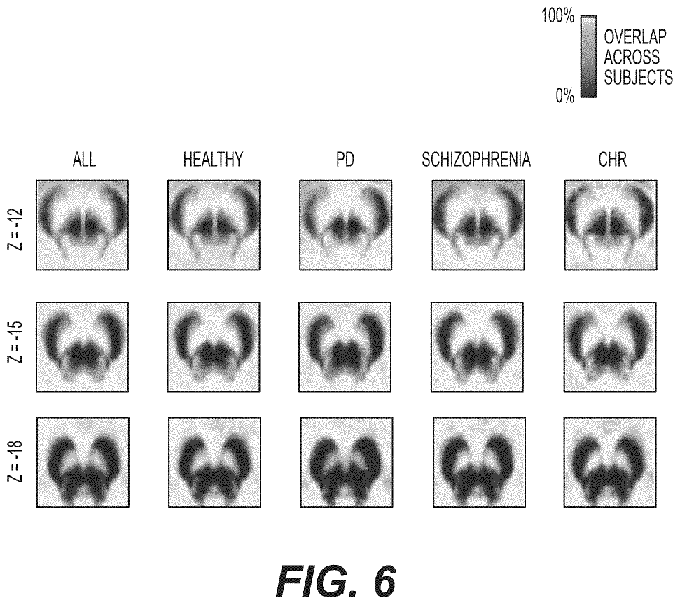

FIG. 6 shows a set of exemplary images of a quality check of the spatial normalization procedures according to an exemplary embodiment of the present disclosure. The exemplary quality check was performed for spatial normalization procedures for all study groups. Overlap images indicate the percentage of subjects with spatially overlapping signal in SN and outside the midbrain for a superior (z=−12), middle (z=−15), and inferior (z=−18) slices (row, from top to bottom), by group (column). These images were generated by creating binary maps of each subject's preprocessed NM-MRI image (e.g., thresholded at CNR=10%) and calculating the percent overlap for each voxel across all subjects in each specific group. As shown in the images of FIG. 6, PD is Parkinson's disease, and CHR is clinical high-risk individuals.

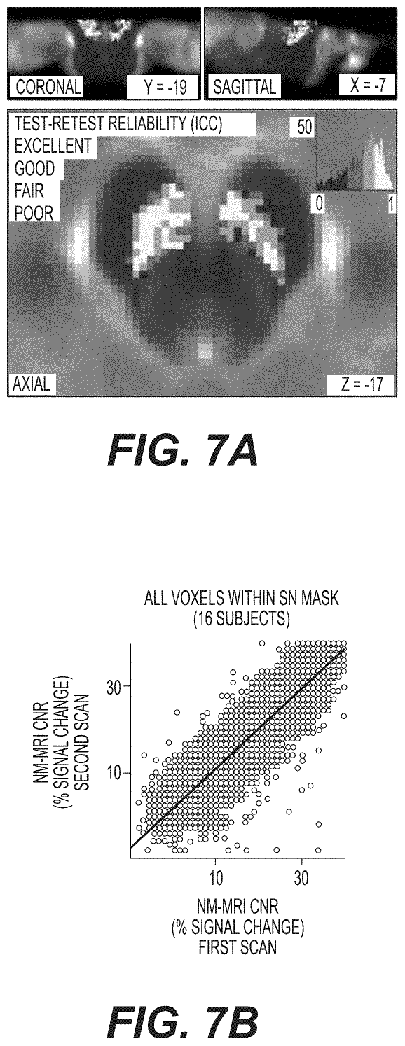

FIG. 7A shows an exemplary map of ICC values across voxels in the SN according to an exemplary embodiment of the present disclosure. The exemplary map of ICC values across voxels in the SN was derived from 2 scans obtained approximately 1 hour apart in the same day (e.g., each of the 2 scans was obtained in 16 subjects). Two-way mixed, single score ICC value was calculated (ICC(3,1); (12)) for each voxel. This ICC score reflects consistency across the first and second scans. Standard thresholds were used for interpretability of ICC values: “excellent” reliability for ICC over 0.75, “good” reliability for ICC between 0.75 and 0.6. “fair” reliability for ICC between 0.6 and 0.4. and “poor” reliability for ICC under 0.4 (13). The inset histogram shown in FIG. 7A shows distribution of ICC (e.g., x axis) values across all in-mask SN voxels (e.g, y axis indicates voxel count). The median ICC across voxels was 0.64 (e,g., 0.35 interquartile range). The ICC for NM-MRI averaged in the whole ICC mask was ‘excellent’ (e.g., ICC =0.95). Calculating absolute agreement across scans for each voxel yielded very similar values (e.g., the median ICC (2,1) was 0.63, 0.35 interquartile range). Note that voxels with poor reliability tend to lie around the edge of the SN mask, which by definition may include voxels outside of SN proper in some subjects.

FIG. 7B shows an exemplary scatterplot showing agreement in NM-MRI CNR for all voxels and all subjects between two scans according to an exemplary embodiment of the present disclosure. The exemplary scatter plot shown in FIG. 7B indicates agreement in NM-MRI CNR for all voxels and all subjects between the 2 scans. This sample consisting of 8 healthy individuals and 8 patients with schizophrenia, with mean age 33.8±13.3 years (a subset of the participants in the current study), was collected as part of a separate study.

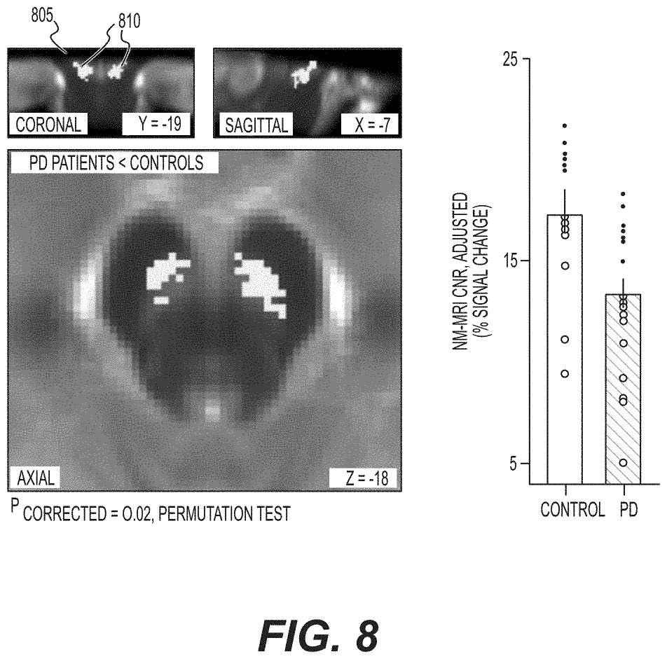

FIG. 8 shows an exemplary map and graph illustrating a comparison of PD patients and matched controls according to an exemplary embodiment of the present disclosure. Map 805 indicates SN voxels where PD patients had decreased NM-MRI CNR (e.g., voxels 810: thresholded at p<0.05, voxel-level), overlaid on the NM-MRI template image. The combined scatterplot and bar plot shows mean NM-MRI CNR values extracted from the voxels plotted by diagnostic group (e.g., PD patients versus sample of matched, healthy controls) for visualization purposes. Each data point is one subject. Error bars are means and SEM. The Cohen's d of size for the group difference in the plotted data was d=1.08. but note that this estimate is biased due to circular voxel selection; the unbiased effect-size was d=0.89 as calculated via a leave-one-out procedure for unbiased voxel selection.

### Exemplary Validation of Voxelwise Approach

Having determined that NM-MRI measures regional concentration of NM in and around the SN, whether regional differences in NM-MRI signal capture biologically meaningful variation across anatomical subregions within the SN was determined. This was performed to interrogate dopamine function, since the heterogeneity of cell populations in the SN (see, e.g., References 22-26) indicates that dopamine function can differ substantially between neuronal tiers projecting to ventral striatum, dorsal striatum or cortical sites. The exemplary system, method and computer-accessible medium, according to an exemplary embodiment of the present disclosure, was used to determine that a voxelwise analysis within the SN can be sensitive to processes affecting specific subregions or likely discontiguous neuronal tiers within the SN (see, e.g., Reference 23) (see, e.g, FIGS. 2A-2C and 6 for information regarding spatial normalization and anatomical masks used in voxelwise analyses). The majority of individual SN voxels exhibited good-to-excellent test-retest reliability (see, e.g., FIGS. 7A and 7B), extending similar demonstrations at the region level. (See, e.g., Reference 30).

To test the anatomical specificity of the voxel-wise NM-MRI approach, the ability of NM-MRI to detect neurodegeneration in PD and the known topography of cell loss in the illness was utilized. Previous PD work has shown decreases in NM concentration (see, e.g., References 16 and 17) and in NM-MRI signal in the whole SN (see, e.g., References 8 and 15) and lateral regions of bisected SN. (See, e.g., References 12-14). Histopathological studies of the SN further support a topographical progression of PD pathology that preferentially affects lateral, posterior, and ventral subregions of SN in mild-to-moderate disease stages. (See, e.g., References 27 and 28). Using NM-MRI data in 28 patients diagnosed with mild-to-moderate PD and 12 age-matched controls, whether a voxelwise analysis would capture this topographic pattern was analyzed. PD patients had significantly lower NM-MRI CNR compared to control individuals (e.g., 439 of 1807 SN voxels at p<0.05, robust linear regression adjusting for age and head coil; p_{corrected} =0.020, permutation test; peak voxel MNL coordinates [x, y, z]: −6, −18, −18 mm; and FIG. 8).

FIG. 3A shows an exemplary set of raw NM-MRI images of the midbrain according to an exemplary embodiment of the present disclosure. FIG. 3B shows an exemplary image and T-statistic maps of the SN showing the size of the signal decrease in NM-MRI CNR in PD compared to matched controls according to an exemplary embodiment of the present disclosure. The exemplary system, method and computer-accessible medium, according to an exemplary embodiment of the present disclosure, was able to capture the known anatomical topography of dopamine neuron loss within the SN (see, e.g., References 27 and 28) (see, e.g., image shown in FIG. 3B): larger CNR decreases in PD tended to predominate in more lateral (β_{|x|}=−0.13, t₁₈₀₃=−14.2, p=10⁻⁴³), posterior (β_{y}−0.05, t₁₈₋₃ =−6.6, p=10⁻¹⁰), and ventral SN voxels (β_{z}=0.17, t₁₈₀₃=16.3, p=10⁻⁵⁵; multiple-linear regression analysis predicting t statistic of group difference across SN voxels as a function of their coordinates in x [absolute distance from the addible], v, and z directions: omnibus F_{3,1803}=111, p=10⁻⁶⁵).

### Exemplary Relationship of NM-MRI Signal to Dopamine Function

Having validated the anatomical sensitivity of the exemplary voxelwise approach, whether NM-MRI signal in the SN correlated with dopamine function in vivo was analyzed. PET imaging was used to measure dopamine release capacity (e.g., ΔBP_{ND}) as the change in D2/D3 radiotracer [¹C] raclopride binding potential between baseline and following administration of dextro-amphetamine (0.5 mg/kg, p.o.). This was used to measure the release of dopamine from the presynaptic sites of dopamine axons, including its vesicular and cytosolic pools (see, e.g., References 31 and 32), into striatal synapses, so it can be relevant that trait-like inter-individual differences in the magnitude of these dopamine pools can be a determinant of NM accumulation. (See, e.g., References 1.9 and 31). Data in a group of 18 individuals without neurodegenerative illness was collected, which included 9 healthy controls and 9 unmedicated patients with schizophrenia. Dopamine release in the associative striatum, part of the dorsal striatum, was focused on to ensure sufficient variability, as patients with schizophrenia tend to exhibit the greatest excess in dopamine release in this subregion. (See, e.g., Reference 33). Also, the dorsal striatum receives projections from the SN (e.g., via the nigrostriatal pathway) while the ventral striatum receives projections predominantly from the ventral tegmental area (e.g., via the mesolimbic pathway) (see, e.g., References 22 and 23), which can be more difficult to visualize in NM-MRI scans due to its lower NM concentration (See. e.g., Reference 16) and smaller size.

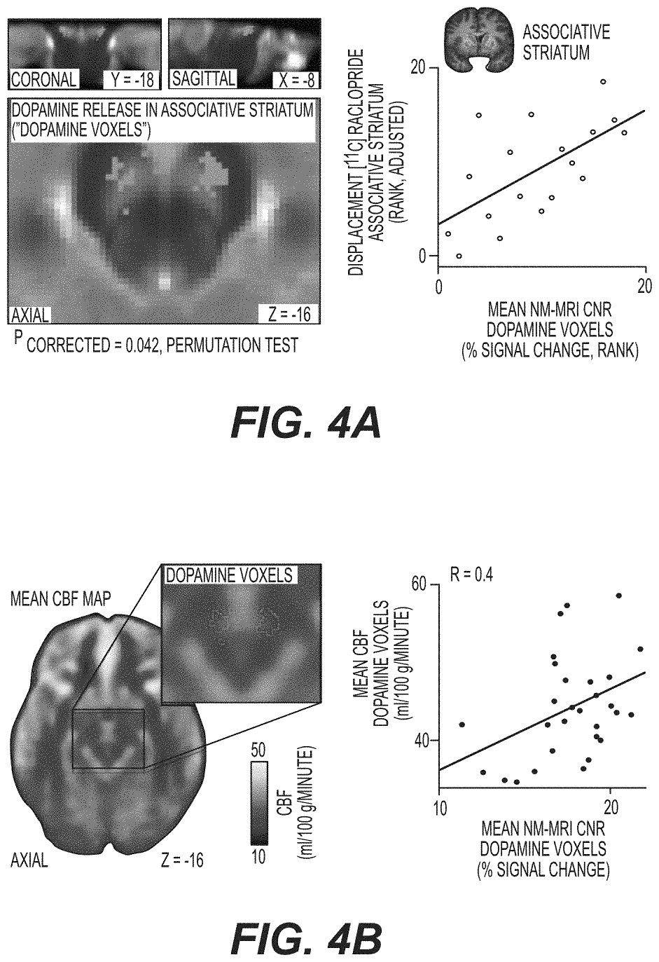

FIG. 4A shows an exemplary image and graph of SN voxels where NM-MRI CNR positively correlated with a PET measure of dopamine release capacity in the associative striatum overlaid on the NM-MRI template image according to an exemplary embodiment of the present disclosure. FIG. 4B shows an exemplary map and graph of a mean resting cerebral blood flow according to an exemplary embodiment of the present disclosure. A voxel wise analysis was performed in which, for each subject, ΔBP_{ND} was measured and correlated to NM-MRI CNR in the SN mask at each voxel. This resulted in a set of SN voxels where NM-MRI CNR correlated positively with dopamine release capacity in the associative striatum (e.g., 225 of 1341 SN voxels at p<0.05, Spearman partial correlation adjusting for diagnosis, age, and head coil; p_{corrected}=0.042, permutation test; peak voxel MNI coordinates [x, y, z]: −1, −18, −16 mm; see, e.g., images and associated graph shown in FIG. 4A). This exemplary effect exhibited. a topographic distribution such that voxels related to dopamine release tended to predominate in anterior and lateral aspects of the SN. This analysis was performed in a smaller SN mask (e.g., 1341 voxels) because relatively few subjects had usable data in the dorsal-most SN. No interaction with diagnosis was found (e.g., p=0.31). The voxelwise results were mirrored by region-of-interest (“ROI”) results showing that mean NM-MRI CNR across the whole SN correlated with mean ΔBP_{ND} in the whole striatum (e.g., p=0.64, p=0.013; partial correlation with the same covariates as the voxelwise analysis and an additional covariate for incomplete SN coverage).

### Exemplary Relationship of NM-MRI Signal to Neural Activity in the SN

Since the latter results indicated that individuals with higher dopamine release from nigrostriatal SN neurons had higher NM accumulation, as measured via NM-MRI, the exemplary system, method and computer-accessible medium was used to determine that NM accumulation can also correlate with local trait-like tendency for increased activity in SN neurons. To test this, arterial spin labeling functional magnetic resonance imaging (“ASL-fMRI”) was used to measure regional cerebral blood flow (“CBF”), a well-established (e.g, indirect) functional measure of neuronal activity (see, e.g., References 34-37) that captures trait-like inter-individual differences in resting activity. (See, e.g., Reference 38). Among 31 individuals without neurodegenerative illness (e.g., 12 healthy individuals, 19 schizophrenia patients) higher CBF in the SN correlated with higher SN NM-MRI CNR. This was true in ROI analyses averaging values in SN voxels related to dopamine release capacity (e.g., “dopamine voxels”, r=0.40, p=0.030; partial correlation controlling for age and diagnosis; see, e.g., CBF maps and associated graph shown in FIG. 4B) and in the whole SN (e.g., r=0.48, p=0.008; partial correlations controlling, for age, diagnosis, and incomplete SN coverage). Again, no interactions with diagnosis were found (e.g., all p>0.7).

### Exemplary Relationship of NM-MRI to Psychosis

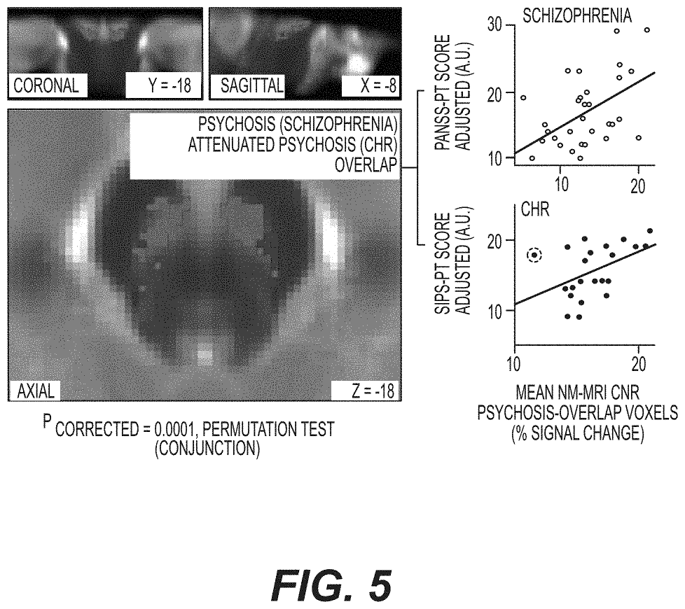

Psychosis can be associated with excessive dopamine release capacity and dopamine synthesis capacity in the striatum (see, e.g., References 23 and 33) in the absence of neurodegeneration of SN neurons. (See, e.g., References 24 and 29). This dopamine dysfunction can be particularly prominent in the associative striatum—which receives projections from discontiguous regions of dorsal and ventral SN tiers through the nigrostriatal pathway (see, e.g., Reference 23) and can be present in schizophrenia (see, e.g. Reference 33), at-risk populations for psychosis (see, e.g., References 39 and 40), and bipolar disorder (see, e.g., Reference 41). indicating a dimensional relationship to psychotic symptoms rather than a specific relationship to schizophrenia or other diagnostic categories. Given this and the evidence presented above supporting that NM-MRI signal indexes dopamine function, it was determined that excess dopamine in SN neurons can result in more accumulation of NM (e.g., in the body of those neurons in the SN (see, e.g., Reference 3)) in individuals with more severe syndromal or subsyndromal psychotic symptoms (e.g., among patients with schizophrenia and among individuals at clinical high-risk [CHR] for psychosis, respectively), Indeed, it was found that more severe (e,g., syndromal) psychotic symptoms in patients with schizophrenia (“PANSS-PT” scores, n=33) and more severe attenuated (e.g. subsyndromal) psychotic symptoms in CHR individuals (e.g., SIPS-PT scores, n=25) both correlated with higher NM-MRI CNR in overlapping SN voxels (e.g., 45 voxels; p_{corrected}=0.00001, permutation test for conjunction effect; and FIG. 5).

FIG. 5 shows an exemplary image and a set of graphs showing how NM-MRI CNR correlates with the severity of psychotic symptoms according to an exemplary embodiment of the present disclosure. The effect shown exhibited a topographic distribution such that psychosis-overlap voxels tended to predominate in ventral and anterior aspects of the SN. Correlations between NM-MRI CNR in these psychosis-overlap voxels and severity of psychosis were specific to positive symptoms of psychosis in schizophrenia (e.g., r=0.38, p=0.044) and CHR (e.g., r=0.57, p=0.006, partial correlations controlling for negative-symptom scores [PANSS-NT or SIPS-NT, respectively], general-symptom scores [PANSS-GT or SIPS-GT, respectively], age, and head coil). Based on the exemplary calibrations with measurements of NM concentration in post-mortem tissue, the estimated difference of NM concentrations in the psychosis-overlap voxels between individuals with the least severe versus the most severe psychotic symptoms would be 0.38 μ/mg versus 0.67 μg/mg in schizophrenia (e.g., estimated concentrations for PANSS-PT scores of 10 versus 29) and 0.31 μg/mg versus 0.62 μg/mg in CHR (e.g., estimated concentrations for SIPS-PT scores of 9 versus 21). Although the exemplary system, method, and computer-accessible medium was used to identify correlates of psychosis rather than of diagnostic categories, the groups were also compared, and no significant differences between the schizophrenia and CHR groups or between either of these groups and matched healthy control groups were found, which is consistent with the notion that the nigrostriatal-dopamine phenotype—at least as captured by NM-MRI—represents a dimensional correlate of psychosis rather than a categorical correlate of diagnosis.

Although a significant overlap between psychosis-overlap voxels and those exhibiting a correlation with dopamine release capacity in the associative striatum (e.g., 6 voxels; p_{corrected}=0.62, permutation test for conjunction) were not found, a significant overlap between voxels where NM-MRI CNR correlated with psychosis in schizophrenia and voxels Where NM-MRI CNR correlated with dopamine release capacity in this striatal subregion (e.g, 80 voxels; p_{corrected}=0.002, permutation test for conjunction) was also found. This indicates that, for example, syndromal psychosis relates to increased NM accumulation in portions of the SN where NM accumulation specifically reflects increased dopamine in the nigrostriatal pathway.

### Exemplary Discussion

NM-MRI as a measure of NM concentration in the SN can be used beyond its use as a marker of neuronal loss in neurodegenerative illness. Consistent with previous preclinical work showing that increased dopamine availability in SN dopamine neurons results in NM accumulation in the soma (see, e.g., References 18 and 19), it was found that an in vivo molecular-imaging readout of dopamine function in these neurons (e.g., striatal dopamine release capacity) correlates with NM-MRI signal in a subregion of the SN among humans without neurodegenerative illness. Cerebral blood flow in the same subregion of the SN also correlated with local increases of NM-MRI CNR, similarly consistent with a link between neural activity in SN and NM accumulation. Altogether, converging evidence from various experiments and different datasets strongly indicates that NM-MRI signal in the SN provides a proxy measure for function of dopamine neurons in this midbrain region, particularly in neuronal tiers of the SN that project to the dorsal striatum via nigrostriatal pathway. (See, e.g., References 22 and 23).

The exemplary system, method, and computer-accessible medium can use NM-MRI measures against a number of gold-standard and well-validated methods (e.g. including high-quality biochemical (see, e.g., Reference 17), PET imaging (see, e.g., References 42 and 43), and clinical measurements (see, e.g., References 44 and 45)) and developed an automated method for regional interrogation of NM-MRI signal within the SN. First, the exemplary post-mortem experiment employed a novel approach for accurate determination of NM concentration across multiple tissue sections throughout the midbrain, which facilitated the confirmation of the ability of NM-MRI to measure regional concentration of NM and to calibrate the NM-MRI signal in subsequent in vivo studies in line with previous recommendations. (See, e.g., Reference 17). Prior work has shown that the NM-MRI contrast mechanism in synthetic NM phantoms depends on the influence of iron-bound melanic NM components on T1-relaxation time and magnetization-transfer ratio (see, e.g., References 9 and 11) and that NM-MRI signal in post-mortem tissue correlates with the density of NM-containing neurons in the SN. (See, e.g., References 46 and 47). The exemplary approach showed that the NM-MRI signal reflects the concentration of NM in tissue, rather than solely reflecting the presence or number of NM-containing SN neurons. Because this observation was apparent in the absence of neurodegeneration of SN neurons, the exemplary system, method and computer-accessible medium use NM-MRI measures of NM concentration, which can be used as a proxy for dopamine function. Second, an exemplary voxelwise method. was utilized that was validated in a cohort of patients with PD (see, e.g., References 8, 10, and 12-15), exhibited a robust reduction of SN CNR, by showing that the exemplary method further revealed a regional pattern of SN signal reduction consistent with the known topographical pattern of neuronal loss in the disease. (See, e.g., References 27 and 28).

The exemplary voxelwise procedure may not only increase the precision and sensitivity of NM-MRI measures but, by virtue of using a standardized space, can also minimize circularity in ROI definitions (see, e.g., Reference 10) and spatial variability between subjects and studies. A correlation between NM-MRI measures again well-validated measure of dopamine function in vivo was established. A PET measure of amphetamine-induced dopamine release which can be thought to reflect the available pools of vesicular and cytosolic dopamine in pre-synaptic dopamine neurons projecting to the striatum. This measure was well-suited to build on preclinical evidence that increased availability of cytosolic dopamine drives NM accumulation. (See, e.g., References 18 and 19). Prior work using different PET dopamine measures in a small sample of young healthy individuals found a correlation between NM-MRI measures and dopamine D2-receptor density in the SN, but not to dopamine synthesis capacity in the midbrain (e.g., via a DOPA measure). (See, e.g., Reference 48). However, such a small and homogeneous sample of young individuals can be unlikely to show substantial variability in dopamine function or NM accumulation and can have thus hampered the sensitivity in detecting an effect, an issue that was circumvented by including individuals with a larger age range and sonic with dopamine dysfunction (e.g., patients). Limitations of PET measures of DOPA in the midbrain (see, e.g., Reference 49) could have also played a role.

By showing that NM-MRI can capture the established dopamine dysfunction associated with psychosis, convergent evidence for the relationship between NM-MRI and dopamine function in the nigrostriatal pathway as well as support for its potential value as a research tool and candidate biomarker for psychosis was shown. Because post-mortem studies have found normal counts of SN dopamine neurons in psychotic patients (see, e.g., References 24 and 29) together with abnormal markers of dopamine function in these neurons (see, References 24, and 50-51) (but see (see, e.g., Reference 52)), increased NM-MRI signal in more severely psychotic individuals likely reflects psychosis-related alterations in dopamine function. This interpretation can also be consistent with PET research in psychosis, which has reliably identified robust increases in dopamine tone in presynaptic dopamine neurons projecting to the striatum, and in particular in nigrostriatal neurons projecting to the dorsal associative striatum. (See, References 23 and 33). This phenotype has been identified in patients with psychotic disorders—including schizophrenia and bipolar disorder—in proportion to the severity of their psychotic symptoms, (See, e.g., References 41 and 53). This dopamine phenotype has also been reported in individuals at high risk for psychosis, particularly in those who go on to develop a psychotic disorder. (See, e.g., References 39 and 40).

The exemplary procedure indicates that the psychosis-related phenotype consisting of .nigrostriatal dopamine excess results in an increase in NM accumulation in the SN that can be captured with NM-MRI. Specifically, a mostly ventral SN subregion was found where NM-MRI CNR can be increased in proportion to severity of psychosis in schizophrenia and to severity of attenuated psychosis in CHR individuals. This mostly ventral subregion of SN (e.g., at least as defined in patients with schizophrenia alone) exhibited a relationship to dopamine. function in the dorsal associative striatum, consistent with the dense projections of the ventral SN tier to this striatal region. (See, e.g., Reference 23). Exploratory analyses failed to detect group differences in NM-MRI CNR between CHR individuals, patients with schizophrenia, and healthy individuals. Consistent with other evidence that dopamine dysfunction can be more closely related to psychosis than to schizophrenia (see, e.g., References 41 and 53), the exemplary data thus supports that NM-MRI captures a psychosis-related (e.g., but not necessarily diagnosis-specific) dysfunction in the nigrostriatal dopamine pathway, with this phenotype antedating the development of full-blown schizophrenia. In contrast, some previous studies found a significant increase in NM-MRI CNR in individuals with schizophrenia (see, e.g. References 20 and 21) that see (see, e.g. References 54 and 55)) but failed to observe a significant relationship between NM-MRI signal and severity of psychotic symptoms. (See, e.g., References 20 and 55). This inconsistency can be explained by the inclusion of patients treated with antidopaminergic medication in these studies. Inclusion of medicated patients can be likely to mask dopaminergic correlates of psychotic symptoms, perhaps by exposing treatment-refractory patients in whom non-dopaminergic alterations can predominate (see, e.g., Reference 56) or perhaps via direct effects of antipsychotic medication on NM accumulation, as some antipsychotics can accumulate in NM organelles (see, e.g., Reference 57) and exhibit a dose-dependent relationship with NM-MRI signals. (See, e.g., Reference 21).

The exemplary findings further underscore the use of NM-MRI as a clinically useful biomarker for non-neurodeszenerative conditions associated with dopamine dysfunction. Such biomarker can have the advantages of being practical (e.g., inexpensive and non-invasive), particularly for pediatric and longitudinal imaging, and of providing high anatomical resolution compared to standard molecular imaging methods, which facilitates it to resolve functionally distinct SN tiers with different pathophysiological roles. (See, e.g., References 22-26). The ability of NM-MRI to index long-term dopamine function, given the slow accumulation of NM in the SN over the lifespan (see, e.g., Reference 17), and the high reproducibility of this procedure (see, e.g., Reference 30) indicates that NM-MRI can be a stable marker insensitive to acute states (e.g., recent sleep loss or substance consumption). This can be a particularly appealing characteristic for a candidate biomarker and one that could complement other markers such as PET-derived measures, which in contrast can better reflect state-dependent dopamine levels. (See, e.g., Reference 53). The lack of significant differences between schizophrenia or CHR and health, together with the observed correlation with severity of psychosis, indicates that NM-MRI better captures a longer-term propensity for psychosis (e.g., compared to more acute psychosis-related states captured by PET measures of dopamine function). Regardless of this, a dimensional marker of psychosis-related dopamine dysfunction can be extremely helpful as a risk biomarker of psychosis. Such a biomarker could further help select a subset of at-risk individuals who, more so than CHR individuals as a whole (see, e.g., References 58 and 59), and can benefit from anti-dopaminergic medication, thus augmenting current risk-prediction procedures based solely on non-biological measures. (See, e.g., Reference 60). NM-metal complexes can also accumulate from oxidation of norepinephrine in the locus coeruleus (see, e.g., References 7 and 61), a nucleus relevant to stress and anxiety disorders (see, e.g., References 62 and 63) as well as to PD and Alzheimer's disease. (See, e.g., Reference 64). The exemplary findings supporting NM-MRI signal in the SN as a measure of dopamine function indicate that NM-MRI signal in the locus coendeus can be a measure of norepinephrine function.

### Exemplary NM-MRI Acquisition

MR images were acquired for all study participants on a GE Healthcare 3T MR750 scanner using a 32-channel, phased-array Nova head coil. A few scans (e.g., 17% of all scans, 24 out of a total of 139) were acquired using an 8-channel in vivo head coil instead. During piloting, various NM-MRI sequences were compared to achieve optimal CNR in the SN using a 2D gradient response echo sequence with magnetization transfer contrast (2D GRE-MT) (see, e.g., Reference 67) with parameters: repetition time (TR)-260 ms, echo time (TE)=2.68 ms, flip angle=40°, in-plane resolution-0.39×0.39 mm², partial brain coverage with field of view (FoV)=162×200, matrix=416×512, number of slices=10, slice thickness=3 mm, slice gap=0 mm, magnetization transfer frequency offset=1200 Hz, number of excitations [NEX]=8, acquisition time=8.04 minutes. The slice-prescription protocol consisted of orienting the image stack along the anterior-commissure-posterior-commissure (“ACPC”) line and placing the top slice 3 mm below the floor of the third ventricle, viewed on a sagittal plane in the middle of the brain. This protocol provided coverage of SN-containing portions of the midbrain (e.g., and cortical and subcortical structures surrounding the brainstem) with high in-plane spatial resolution using a short scan easy to tolerate by clinical populations. Whole-brain, high-resolution structural MRI scans were also acquired pre-processing of the 2D GRE-MT (e.g., NM-MRI) data: a T1-weighted 3D BRAVO sequence (e.g., inversion time=450 ms, TR≈7.85 ms, TE≈3.10 ms, angle=12°, FoV-240×240, matrix-300×300, number of slices=220, isotropic voxel size=0.8 mm³) and a T2-weighted CUBE sequence (e.g., TR=2.50 ms, TE≈0.98 ms , echo train length=120, FoV=256×256, number of slices=1, isotropic voxel size=8 mm³). The quality of NM-MRI images was visually inspected for artifacts immediately upon acquisition, and scans were repeated when necessary, time permitting. Ten participants were excluded due to clearly visible smearing or banding artifacts affecting the midbrain (e.g., due to participant motion, n=4), or incorrect imaging-stack placement (e.g., n=6).

### Exemplary NM-MRI Preprocessing

NM-MRI scans were preprocessed using SPM12 to facilitate voxelwise analyses in standardized MNI space. For example, NM-MRI scans and T2-weighted scans were coregistered to T1-weighted scans. Tissue segmentation was performed using T1- and T2-weighted scans as separate channels (e.g., segmentation was performed based solely on the T1 weighted scan for 15 psychosis controls, 1 PD patient, and 2 schizophrenia patients missing T2-weighted scans). Scans from all study participants were normalized into MNI space using DARTEL routines (see, e.g., Reference 68) with a gray- and white-matter template generated from an initial sample of 40 individuals (e.g., 20 schizophrenia patients and 20 controls). The resampled voxel size of unsmoothed, normalized NM-MRI scans was 1 mm, isotropic. All images were visually inspected following each preprocessing procedure. (See, e.g., FIGS. 2C and 6 for quality checks of spatial normalization). Intensity normalization and spatial smoothing were then performed using custom Matlab scripts. CNR for each subject and voxel v was calculated as the relative change in NM-MRI signal intensity I from a reference region RR of white-matter tracts known to have minimal NM content, the cerebri, as CNR_{V}=(Iᵥ−mode(I_{RR}))/mode(I_{RR}).

FIG. 2A show an exemplary Template NM-MRI image created by averaging the spatially normalized NM-MRI images according to an exemplary embodiment of the present disclosure. FIG. 28 shows an exemplary image of masks for the substantia nigra and the crus cerebri reference region according to an exemplary embodiment of the present disclosure. FIG. 2C shows a set of exemplary 3D images and signal change diagrams according to an exemplary embodiment of the present disclosure;

A template mask of the reference region in MNI space (see, e.g., image shown in FIG. 2B) was created by manual tracing on a template NM-MRI image (e.g., an average of normalized NM-MRI scans from the initial sample of 40 individuals, see, for example, image shown in FIG. 2A). The mode(I_{RR}) was calculated for each participant from kernel-smoothing-function fit of a histogram of all voxels in the mask. The mode rather than mean or median was utilized because it was found it to be more robust to outlier voxels (e.g., due to edge artifacts) and this precluded the need for further modification of the reference-region mask. Images were then spatially smoothed with a 1-mm full-width-at-half-maximum Gaussian kernel.

Further, an over inclusive mask of SN voxels was created by manual tracing on the template NM-MRI image. The mask was subsequently reduced by eliminating edge voxels with extreme values: voxels showing extreme relative values for a given participant (e.g., beyond the 1^{st} or the 99^{th} percentile of the CNR distribution across SN voxels in more than 2 subjects) or voxels that had consistently low signal across participants (e.g., CNR less than 5% in more than 90% of subjects). These procedures removed 9% of the voxels in the manually traced mask, leaving a filial template SN mask containing 1,807 resampled voxels. (See, e.g., image shown in FIG. 28).

### Exemplary NM-MRI Analysis

All analyses were carried out in Matlab (Mathworks, Natick, Mass.) using custom scripts. In general, robust linear regression analyses were performed across subjects for every voxel v within the SN mask, as: CNRᵥ=β₀+β₁·measure of interest++Σᵢ₌₂ⁿβᵢ·nuisance covariate+ε. The measure of interest consisted of either imaging (e.g., dopamine release capacity) or clinical (e.g., psychosis severity) data, depending on the analysis. Nuisance covariates, including diagnosis, head coil, and age, varied for different analyses; while all analyses included an age covariate, head coil and diagnosis covariates were only included in analyses where these variables differed across subjects. Robust linear regression was used to minimize the need for regression diagnostics in the context of mass-univariate, voxelwise analyses. A partial (e.g., non-parametric) Spearman correlation was used instead of linear regression if variables were not normally distributed according to a Lilliefors test at p<0.05 (e.g., which was the case for dopamine release capacity). Voxelwise analyses were carried out within the template SN mask after censoring subject data points with missing values (e.g., due to incomplete coverage of the dorsal SN in a minority of subjects resulting from inter-individual variability in anatomy) or extreme values (e.g., values more extreme than the 1^{st} or the 99^{th} percentile of the CNR distribution across all SN voxels and subjects [CNR values below −9% or above 40%, respectively]). For all voxelwise analyses, the spatial extent of an effect was defined as the number of voxels k (e.g., adjacent or non-adjacent) exhibiting a significant relationship between the measure of interest and CNR voxel-level height threshold for t-test of regression coefficient β₁ of p<0.05one-sided [β₁⁺]).

Hypothesis testing was based on a permutation test in which the measure of interest was randomly shuffled with respect to CNR. This test corrected for multiple comparisons by determining whether an effect's spatial extent k was greater than would be expected by chance (e.g., p_{corrected}<0.05, 10,000 permutations; equivalent to a cluster-level family-wise-error-corrected p-value, although in this case voxels were not required to form a cluster of adjacent voxels, given the small size of the SN and evidence that SN tiers defined by specific projection sites do not necessarily comprise anatomically clustered neurons). (See, e.g., Reference 23). On each iteration, the order of the values of a variable of interest (e.g. dopamine release capacity) was randomly permuted across subjects (e.g., and maintained for the analysis of every voxel within the. SN mask for a given iteration of the permutation test, accounting for spatial dependencies). This provided a measure of spatial extent for each of 10,000 permuted datasets, forming a null distribution against which to calculate the probability of observing the spatial extent k of the effect in the true data by chance (p_{corrected}). For hypothesis testing related to conjunction effects (e.g., overlap of psychosis effects in the two clinical groups), permutation analysis determined if the extent k of overlap for both effects (β₁_{_{effect1}}⁺∩β₁_{_{effect2}}⁺) was greater than would be expected by chance (e.g., p<0.05, 10,000 permutations) based on a null distribution counting the overlap of significant voxels after the location of true significant voxels for each effect was randomly shuffled within the SN mask.

Exemplary Topographical analyses. Multiple-linear regression analysis across SN voxels was used to predict the strength of an effect (e.g., or the presence of a significant conjunction effect) as a function of MNI voxel coordinates in the x (e.g., absolute distance from the midline), y, and z directions.

Exemplary ROI analyses. Post hoc ROI analyses examining mean NM-MRI signal across voxels in the. whole SN mask included the same covariates as used in the respective voxelwise analyses plus an additional dummy covariate indexing subjects with incomplete coverage of dorsal SN, as a dorsal-ventral gradient of signal intensity in SN biased mean CNR values in these subjects. This “incomplete SN coverage” covariate was not used for analyses on NM-MRI signal extracted from “dopamine” voxels or “psychosis-overlap” voxels as these confined sets of voxels had a relatively small contribution from dorsal SN.

### Exemplary Post-Mortem Experiment

Post-mortem specimens of human midbrain tissue were obtained from The New York Brain Bank at Columbia University. Seven specimens were obtained, each from an individual who suffered from Alzheimer's disease or other non-PD dementia at the time of death (e.g., ages 44 to 90). Specimens were approximately 3-mm-thick slices of fresh frozen tissue from the rostral hemi-midbrain containing pigmented SN. These specimens were scanned using the NM-MRI protocol similar to the one used in vivo, after which they were dissected for analyses of NM tissue concentration. The dish containing the specimen included a grid insert, used to keep dissections in register with MR images.

Exemplary Neurochemical measurement of NM concentration in post-mortem tissue. Samples deriving from each grid section were homogenized with titanium tools. NM concentration of each grid. section was then measured according to the exemplary previously described spectrophotometry method (see, e.g., Reference 17), with minor modifications to improve the removal of interfering tissue components from midbrain regions with higher content of fibers and fewer NM-containing neurons compared to sections of SN proper dissected along anatomical boundaries. Additional tests confirmed that the exemplary methods for Fomblin® cleaning were effective and that neither this substance nor the methylene blue dye was likely to influence spectrophotometric measurements of NM. Data from 2% of grid sections (e.g., 2 out of 118) could riot be used due to technical problems with dissection, handling, or measurement.

Exemplary MRI measurement of NM signal in post-mortem tissue. NM-MRI signal was measured in corresponding grid sections using a custom Matlab script. Processing of NM-MRI images included automated removal of voxels showing edge artifacts and signal dropout, averaging over slices to create a two-dimensional (“2D”) image, and registration with a grid of dimensions matching the grid insert. The grid registration was adjusted manually based on the well markers and grid-shaped edge artifacts present in the superior-most slice where the grid insert rested. Signal in the remaining voxels was averaged within each grid section. To normalize signal intensity across specimens, CNR for each grid section was calculated as in the in vivo voxelwise. The reference region for each specimen was defined by the 3 grid sections that best matched the location of the crus cerebri reference region used for in vivo scanning.

Exemplary Statistical analysis of post-mortem data. A generalized linear mixed-effects (“GLME”) model including data across all grid sections g and specimens s was used to predict NM tissue concentration in each aid. section based on mean NM-MRI CNR in the same grid section. GLME analyses used an isotropic covariance matrix and were fitted via maximum pseudo-likelihood estimation, as implemented via the Matlab function fitglme. Likelihood-ratio tests at p<0.05 favored reduced models without random slopes. Therefore, all models included random intercepts but not random slopes, as: [NM]_{gs}β₀+β₁. C̅N̅R̅_{gs}+Σᵢ₌₂ⁿβᵢ·nuisance covariate_{gs}+b_{Os}+ε_{gs}. The basic model only included mean NM-MRI CNR in a given grid section C̅N̅R̅_{gs} as a fixed-effects predictor. Sections near the PAG tended to have relatively high signal intensity but low NM tissue concentration. Thus, an extended model included a binary variable for PAG presence in grid sections (e.g., P.G+, PAG−) and an interaction term of NM-MRI CNR×PAG as additional fixed-effects covariates (e.g., the interaction was significant at p=0.040, confirming that NM-MRI was less strongly related to NM concentration in PAG+ relative to PAG− regions). PAG+ grid sections (e.g., 1 to 5 per specimen) were defined as those situated at the posterior-medial aspect of the specimen and consistent with the anatomical location of the PAG. A control analysis additionally included a fixed-effects covariate, indicating the proportion of voxels containing SN for each grid section, defined as the proportion of voxels with CNR higher than 10% in grid sections deemed to contain SN upon visual inspection. This latter control analysis aimed to test whether regional. variability in NM-MRI CNR can predict regional variability in NM tissue concentration even after accounting for changes in both measures as a mere function of the presence or absence of SN neurons in a given region (e.g., in combination with partial-volume effects).

### Exemplary PET Imaging Procedure

Eighteen (18) subjects without neurodegenerative illness (e.g., 9 healthy controls, 9 unmedicated schizophrenia patients) underwent PET scanning using the radiotracer [¹¹C]raclopride and an amphetamine challenge to quantify dopamine release capacity. All of these subjects also participated in the psychosis study and are described below. A baseline (e.g., pre-amphetamine) PET scan was conducted on one day, and a post-amphetamine PET scan was acquired the next day (see, e.g., Reference 69), 5-7 hours after administration of dextroamphetamine (e.g., 0.5 mg/kg, p.o.). Table 3 below shows PET scan parameters and characteristics of participants in the PET study. For each PET scan, list-mode data were acquired on a Biograph mCT PET-CT scanner (Siemens/CTI, Knoxville Tenn.) over 60 minutes following a single bolus injection of [¹¹C]raclopride, binned into a sequence of frames of increasing duration and reconstructed by filtered back projection using manufacturer-provided software. PET data were motion-corrected and registered to the individuals' T1-weighted MRI scan using SPM2. ROIs were drawn on each subject's T1-weighted MRI scan and transferred to the coregistered PET data. Time-activity curves were formed as the mean activity in each ROI in each frame. The exemplary a priori ROI was the associative striatum, defined as the entire caudate nucleus and the precommissural putamen (see, e.g., References 33 and 70), a part of the dorsal striatum that receives nigrostriatal axonal projections from SN neurons (see, e.g., References 22 and 23) and that has been consistently implicated in psychosis. (See, e.g., Reference 23). Data were analyzed using the simplified reference-tissue model (“SRTM”) (see, e.g. References 71 and 72) with cerebellum as a reference tissue to determine the binding potential relative to the non-displaceable compartment (e.g., BP_{ND}). The primary outcome measure was the relative reduction in BP_{ND} (ΔBP_{ND}), reflecting amphetamine-induced dopamine release, a measure of dopamine release capacity. Amphetamine induces synaptic release of dopamine derived from both cytosolic and vesicular stores. (See, e.g., Reference 31). This results in excessive competition with the radiotracer at the D2 receptor, and, simultaneously, agonist-induced D2-receptor internalization, both of which can cause radiotracer displacement and lower BP_{ND}. (See, References 23 and 73-75). ΔBP_{ND} thus combines both effects and reflects the magnitude of dopamine stores. Since these stores depend on dopamine synthesis, the dopamine release capacity PET measure can be relevant to dopamine function. It can also be relevant to NM given that NM accumulation can be driven by cytosolic dopamine (e.g., or by vesicular dopamine once it can be transported into the cytosol). (See, e.g., References 6, 10, and 19).

### Exemplary Arterial Spin Labeling (“ASL”) Perfusion Imaging Study

Thirty-one (31) subjects without neurodegenerative illness (e.g., 12 healthy controls, 19 schizophrenia patients, 74% male [23/31], mean age 32 years) underwent ASL functional MRI scanning at rest to quantify regional CBF. All of these subjects also participated in the psychosis study and are described below. Pseudo-continuous ASL (e.g., 3D-pCASL) perfusion imaging was performed using a 3D background suppressed fast spin-echo stack-of-spiral readout module with eight in-plane spiral inteleaves (e.g., TR=4463 ms, TE=10.2 ms, labeling duration-1500 ms, post-labeling delay-2500 ms, no flow-crushing gradients, FoV=240×240, NEX-3, slice thickness=4 mm) and an echo train length of 23 to obtain 23 consecutive axial slices. A labeling plane of 10-mm thick was placed 20 mm inferior to the lower edge of the cerebellum. Total scan time was 259 s. The perfusion data were analyzed to create CBF images using Functool software (version 9.4, GE Medical Systems). CBF was calculated as in prior work. (See, e.g., Reference 76).

For preprocessing, CBF images were coregistered to ASL-localizer images, which were then coregistered to T1 images, with the coregistration parameters applied to CBF images. CBF images Were then normalized into MNI space using the same procedures described above for NM-MRI scans. Mean CBF was calculated within the whole SN mask and within the mask of SN voxels significantly related to dopamine release capacity in the associative stratium. ROI-based partial correlation analyses tested the relationship between mean CBF and mean NM-MRI CNR in the same mask, controlling for age and diagnosis.

### Exemplary Psychosis Study

Thirty-three unmedicated patients with schizophrenia and 25 individuals at CHR for psychosis participated in the study. Healthy controls were used for exploratory comparison purposes: one group (e.g., n=30) age-matched to the schizophrenia group and another (e.g., n=15) age-matched to the CHR group. See Tables 2 and 4 for demographic and clinical information for all relevant groups.

### Further Post-Mortem Experiment

Post-mortem specimens of human midbrain tissue were obtained from The New York. Brain Bank at Columbia University. Seven (7) specimens were obtained, each from an individual who suffered. from Alzhemier's disease or other non-PD dementia at the time of death (e.g., ages 44 to 90; for further clinical and demographic information see Table 1 below). None suffered from PD, Parkinsonian syndromes, or any other movement disorder or neurodegenerative illness affecting the SN, based on neuropathological examination for accumulation of abnormal proteins such as alpha-synuclein, beta-amyloid or tau. One case showed marked decrease in neuronal density in the SN despite clearly identifiable NM. Analyses excluding this one case did not change the observed relationship between NM-MRI CNR and NM concentration. Therefore, the data presented include this case to increase statistical power. Specimens were approximately 3-mm-thick slices of fresh frozen tissue from the rostral hemi-midbrain of the right hemisphere containing pigmented SN. They were stored at −80° C. These specimens were scanned using the NM-MRI protocol, after which they were dissected for analyses of NM tissue concentration. For the MRI scanning session, the specimens were progressively thawed to 20° C., as verified via a laser thermometer. Specimens were placed in a custom-made dish 3D-printed from MRI-compatible nylon polymer (NW Rapid Mfg, McMinnville, Oreg.; see, e.g., FIGS. 6A and 6C) and a matching grid-insert lid was placed on top of the specimen and affixed to hold the specimen in place. While secured in the dish, specimens were fully immersed in an MRI-invisible lubricant (Fomblin® perfluoropolyether Y25; Solvay, Thorofare, N.J.) and placed in a desiccator or 30 minutes to remove air from the tissue. Wells in the four cardinal points of the rim of the dish were filled with water to mark its location and orientation in the MRI images. The dishes were then placed on a custom stand inside a 32-channel, phased-array Nova head coil and scanned using the exemplary 2D GRE-MT NM-MRI sequence described above for in vivo imaging. The only changes in the post-mortem scanning protocol were an increase in the resolution (e.g., in-plane resolution 0.3125×0.3125 mm², slice thickness=0.60 mm) and a decrease in the FoV (e.g., 160×80).

After the scanning session, samples were refrozen in place and marked with gridlines by applying methylene blue dye (e.g., 0.05% water solution [5 mg/10 ml]; Sigma-Aldrich, St. Louis, Mo.) to the tissue using the grid insert as a stamp. Guides built into the walls of the dish ensured that the orientation of the grid with respect to the specimen was fixed at all times. Within 4 days post-scanning, partially thawed specimens were dissected along gridlines after extensive removal of Fontblin® by dripping tissue slices, followed by gently roiling the surface of sections on ultractean filter paper. Dissection and manipulation of tissue sections were performed with ceramic blades and titanium-and-plastic forceps to avoid contamination from iron. Each grid section (e.g., 3.5 mm×3.5 mm×approximately 3 mm, depending on the slice thickness), together with any adjacent partial grid sections, was weighed, stored separately in Eppendorf tubes, and frozen. Specimens were thus divided into 13-20 grid sections; the grid column and row number of each dissected grid section was coded.

TABLE 1
Clinical and demographic information
for post-mortem specimens

| | | | | Cold | Frozen |
|---|---|---|---|---|---|
| | | | | post- | post- |
| | | | | mortem | mortem |
| Sample | | | | interval | interval |
| ID | Age | Sex | Diagnosis | (hours) | (hours) |
| 1 | 81 | F | AD | 6.5 | 19.3 |
| 2 | 76 | F | AD | 5.5 | 18.2 |
| 3 | 44 | M | AD | 2.8 | 20.8 |
| 4 | 72 | M | AD | — | 9.0 |
| 5 | >88 | — | AD (probable) | 3.1 | 25.7 |
| 6 | >88 | F | Dementia | 0.8 | 16.5 |
| 7 | 84 | F | AD (possible) | 1.8 | 23.7 |

AD: Alzheimer's disease.
F: female;
M: male.

Exemplary NM-MRI Analysis: Exclusion of Voxels with Few Observations

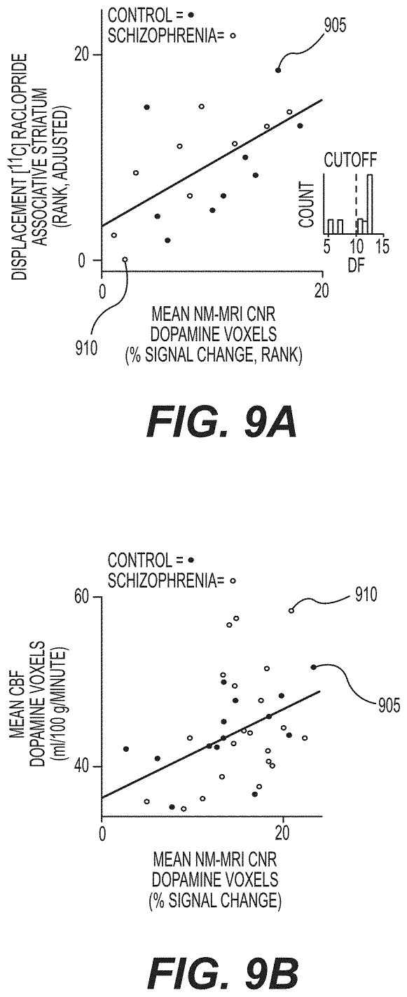

To reduce the risk of type II error, voxels were excluded from the analysis if, after censoring of subject data points with missing or extreme values, the t-test of the regression coefficient β₁ for a particular analysis had fewer than 10 degrees of freedom (e.g., note that the degrees of freedom take into account the sample size with usable data in a given voxel as well as the number of model predictors). This voxel exclusion only applied to the analysis relating NM-MRI signal to dopamine release capacity given the smaller sample size of the PET dataset, and this analysis was thus performed for 1,341 resampled SN voxels (e.g., rather than for the full mask of 1,807 resampled voxels). Selecting exclusion thresholds anywhere between about 8-11 degrees of freedom gave very similar results. See inset shown in the graph of FIG. 9A for distribution of degrees of freedom for all voxels in this analysis.

FIGS. 9A and 9B show exemplary scatterplots illustrating how NM-MRI CNR correlates with measures of dopamine function across individuals without neurodegenerative illness according to an exemplary embodiment of the present disclosure. NM-MRI CNR correlates with measures of dopamine function across individuals without neurodegenerative illness. The scatterplots shown in FIGS. 4A and 4B are shown in FIGS. 9A and 9B, indicating distinct participant groups (e.g., the control group is shown as element 905 and the schizophrenia patients are shown as element 910). No interactions by group were found for either analysis (all p>0.05). The inset histogram in FIG. 9A shows the distribution of degrees of freedom (df; for t-test of the regression coefficient β1) for all analyzed voxels in the voxelwise analysis relating dopamine release capacity to NM-MRI CNR. The presence of some NM-MRI scans lacking full-coverage of the SN (e.g., due to interindividual differences in anatomy) led to reduction in degrees of freedom for some (e.g., more dorsal) voxels. These voxels are represented as the minor mode on the left of the histogram, where all voxels have degrees of freedom below 10. Thus, the cutoff for voxel exclusion was set at df<10. (See e.g., broken line shown in the histogram of FIG. 9A.

### Exemplary NM-MRI Analysis: Non-Circular Voxel Selection for Estimation of Unbiased Effect Size

For voxelwise analyses, an unbiased measure of effect size was generated by using a leave-one-out procedure: for a given subject, voxels where the variable of interest was related to NM-MRI signal were first identified in an analysis including all subjects except for this (e.g., held-out) subject. The mean signal in the held-out subject was then calculated from this set of voxels. This procedure was repeated for all subjects so that each subject had an extracted, mean, NM-MRI signal value obtained from an analysis that excluded them. This unbiased voxel selection and. data extraction thus avoided statistical circularity. Unbiased estimates of effect size (e.g Cohen's d or correlation coefficient) were then determined by relating these extracted NM-MRI signal values to variables of interest across held-out subjects and including the same covariates as in the voxelwise analysis and an additional covariate indexing subjects lacking full dorsal-SN coverage (e.g., due to dorsal-ventral gradient in NM-MRI signal intensity).

### Exemplary Neurochemical Measurement AM Concentration in Post-Mortem Tissue: Examination of Chemical Agents Applied To Post-Mortem Tissue

To test whether Fomblin® influenced NM measurement, small cubes of SN pars compacta with similar levels of pigmentation were dissected from a single healthy subject. Some cubes (e.g, n=3) were immersed in Fomblin®, then cleaned of the Fomblin® (e.g., drained and rolled oil filter paper); the remaining cubes (e.g., n=5) were not immersed in Fomblin® as control samples. NM concentration was comparable in these two sets of cubes (e.g., mean±standard deviation: 0.82±0.08 versus 0.86±0.09 μg NM/mg wet tissue, respectively; t₆=−0.62, p=0.56). The water-soluble methylene blue dye was efficiently removed during washing procedures in the exemplary standard protocol to measure NM concentration; moreover, it was confirmed that the absorption wavelength of this compound (e.g., with a peak near 680 nm) can be far from that used in the determination of NM concentration (e.g., 350 nm).

### Exemplary MRI Measurement of NM Signal in Post-Mortem Tissue: Automated Removal of Voxels Showing Edge Artifacts and Signal Dropout

Processing of NM-MRI images included automated removal of low-signal voxels, including all voxels outside of the specimen or voxels within the specimen showing signal dropout. The threshold for exclusion of low-signal voxels was determined for each specimen based on the histogram of all voxels the image, which was fitted using a kernel smoothing function. The threshold was defined as the signal corresponding to the minimum lying between the leftmost peak in the fitted histogram, corresponding to low-signal voxels outside of the specimen, and the rightmost peak, corresponding to higher-signal voxels within the specimen (e.g., consistent with a bimodal distribution).

To eliminate edge artifacts, the first exemplary procedure was to define the boundaries between the specimen and the surrounding space outside the specimen and between the specimen and areas of signal dropout. These boundaries were defined in 3D and 2D. To do so, boundary voxels of the specimen that lay directly next to low signal voxels, (defined above, were labeled using the bwperim function in Matlab these boundary voxels were defined for the whole volume and also for a 2D flattened image created by averaging over slices. These boundary voxels were removed from the specimen (e.g., first the 3D border voxels were removed from the 3D image, then the 2D boundary voxels, dilated by 2 voxels, were removed from the resulting flattened image). Finally, voxels with extreme signal values (e.g., Cook's distance>4/n in a constant-only linear regression model) relative to other voxels in the same 2D grid section were removed. The resultant 2D image, cleaned of edge artifacts, signal dropout and other outlier voxels, was carried forward to the final analysis procedures.

### Exemplary PET Imaging Study: Timing of Post-Amphetamine PET Scan

Each subject received 2 post-amphetamine PET scans for the purposes of a separate experiment, which was previously published. (See, e.g., Reference 77). This previous study aimed at assessing the time course of receptor internalization after an agonist challenge, measured via prolonged displacement of the D2 radiotracer [¹¹C]raclopride. PET scans were acquired in four sessions: baseline, 3 hours after amphetamine, 5 to 7 hours after amphetamine and 10 hours after amphetamine, However, not all time points post-amphetamine were available for all subjects, Displacement was however highly stable and did not differ between the 3-hours and the 5-7-hour time points (see, e.g., Reference 77) (ΔBP_{ND} indeed strongly correlated across subjects between these two time points; r=0.75). Only one of these post-amphetamine scans was used: the one administered 5-7 hour post-amphetamine. The 5-7-hour time point was selected (see, Reference 78), because this was the time point with the most available data (e.g., missing for only 3/18 participants for whom it was substituted by data from the 3 hour time point). Displacement at 5 to 7 hours post-amphetamine—like displacement at 3 hours post-amphetamine reflects the magnitude of dopamine release due to amphetamine, which can be a combination of competition between dopamine and the radiotracer for binding to the receptor (see, e.g., Reference 79), and agonist-induced receptor internalization, both of which depend on the magnitude of agonist availability. (See, e.g., References 80 and 81). Thus, the 5-7-hour time point can be the optimal time point for this study due to the larger number of subjects with available data and given the observed stability of the displacement between the 3-hour and the 5-7-hour time points. At the 10-hour time point BP_{ND} tended. to be higher, likely due to a decrease in receptor internalization following, recycling of receptors. Examining the 11 subjects with PET data at 3 hours revealed that the effect size of the correlation between NM-MRI CNR and ΔBP_{ND} at this 3 hour time point was similar to that at the 5-7 hour time point.

### Exemplary ASL Perfusion Imaging Study: CBF Calculation

CBF was calculated with the following equation (see, e.g., Reference 82):

$$CBF = 6000 \times λ \frac{( 1 - {e}^{\frac{ST}{{T}_{1 t}}} ) {e}^{\frac{PLD}{{T}_{1 b}}}}{2 {ϵT}_{1 b} ( 1 - {e}^{- \frac{LT}{{T}_{1 b}}} )} ( \frac{PW}{{SF}_{PW} PR} )$$

Here, the longitudinal relaxation time (T1) of blood (T_{1b}) was assumed to be 1.6 s at 3.0T, T1 of tissue (Tₗₜ) 1.2 s, partition coefficient (λ) 0.9, labeling efficiency (ε) 0.6, saturation time (“ST”) 2 s, labeling duration (“LT”) 1.5 s, and post-labeling delay (“PLD”) 1,525 ms. PW can be the perfusion weighted or the raw difference image; PR can be the partial saturation of the reference image, and SF_{PW} can be an empirical scaling factor (e.g., 32) used to increase the dynamic range of the PW.

Exemplary Parkinson's disease study. Twenty-eight patients with idiopathic PD, as per UK Parkinson's Disease Society Brain Bank Criteria, were recruited either from the Center for Parkinson's Disease and other Movement Disorders at the Columbia University Medical Center or from the Michael. J. Fox Foundation Trial Finder website. Patients were in the mild-to-moderate stages of disease (e.g., mean Unified Parkinson's Disease Rating Scale [UPDRS] off-medication score of 30, as administered by a movement disorders neurologist, with an average disease duration of 7.3 years; Table 2 below). All patients had been receiving L-DOPA treatment for at least 6 months. Eleven of the 28 patients were scanned in the off-medication state (e.g., defined as more than 12 hours since the last dose of dopaminergic medication intake). NM-MRI signals did not differ between patients scanned on versus off medication. An age-matched sample of 12 healthy control participants (e.g., 4 of which also participated in the psychosis study described below) was recruited from the local community. (See, Table 2).

TABLE 2
Clinical and demographic characteristics of clinical samples for psychosis and
Parkinson's disease studies.

| | | | | | |
|---|---|---|---|---|---|
| | | | | PD- | |
| | | | | matched | PD vs. |
| | | Clinical | Parkinson's | healthy | matched |
| | schizophrenia | high-risk | disease | controls | controls |
| Characteristic | (n = 33) | (n = 25) | (n = 28) | (n = 12) | (p-value) |

Socio-demographic characteristics

| Age, years [range] | | 33.9 ± 2.2 | 25.6 ± 0.97 | 63 ± 1.1 | 62 ± 2.44 | 0.64 |
|---|---|---|---|---|---|---|
| | | [19-58] | [15-30] | [51-72] | [51-73] | |
| Sex (male) | | 23 (70%) | 13 (52%) | 24 (86%) | 9 (75%) | 0.41 |
| Race/ | African- | 17 | 6 | 0 | 2 | 0.014 |
| ethnicity | American | | | | | |
| | Asian | 2 | 1 | 0 | 2 | |
| | Caucasian | 7 | 10 | 26 | 7 | |
| | Hispanic | 5 | 2 | 2 | 1 | |
| | Mixed | 2 | 6 | 0 | 0 | |

| | | | | | |
|---|---|---|---|---|---|
| Parental SES | 41.7 ± 2.4 | 44.5 ± 2.2 | — | — | — |

Clinical characteristics

| Antipsychotic history | | 17/16 | — | — | — | — |
|---|---|---|---|---|---|---|
| (drug naïve/drug fee)¹ | | | | | | |
| UPDRS | Off | — | — | 30 ± 1.8 | 1.5 ± 0.43 | <0.001 |
| | medication | | | | | |
| | On | — | — | 20 ± 1.5 | — | — |
| | medication | | | | | |
| MoCA | | — | — | 27.4 ± 0.43 | 29 ± 0.34 | 0.18 |
| Disease duration, years | | — | — | 7.3 ± 0.64 | — | — |
| PANSS | Positive total | 16.9 ± 0.93 | — | — | — | — |
| | [range: 7-49] | | | | | |
| | Negative total | 15.4 ± 0.99 | — | — | — | — |
| | [range: 7-49] | | | | | |
| | General total | 32.8 ± 1.8 | — | — | — | — |
| | [range: 16-112] | | | | | |
| SIPS | Positive total | — | 15.6 ± 0.70 | — | — | — |
| | [range: 0-30] | | | | | |
| | Negative total | — | 17.4 ± 0.91 | — | — | — |
| | [range: 0-36] | | | | | |
| | Disorganization | — | 11.2 ± 0.53 | — | — | — |
| | total | | | | | |
| | [range: 0-24] | | | | | |
| | General total | — | 13.7 ± 0.68 | — | — | — |
| | [range: 0-24] | | | | | |

Means ± standard error are given for continuous variables; number (and percentage) are given for categorical variables. P-values for group comparison of Parkinson’s disease patients and healthy controls are given based on two-sample t-tests for continuous variables and X² tests for categorical variables.
¹Antipsychotic medication status was considered “drug-naïve” if lifetime exposure <6 weeks and none in past 3 weeks, and “drug-free” if none in past 3 weeks.
Parental SES: parental socio-economic status as measured via the Hollingshead scale.
UPDRS: Unified Parkinson’s Disease Rating Scale.
MoCA: Montreal Cognitive Assessment.
PANSS: Positive and Negative Syndrome Scale (positive or psychotic symptoms of schizophrenia include hallucinations and delusions; negative symptoms include emotional withdrawal and amotivation).
SIPS: Structured Interview for Prodromal Syndromes.
See Supplement for information on study participants for other studies.

Exemplary Schizophrenia sample. The inclusion criteria were: age 18-55 years; DSM-FV criteria for schizophrenia, sehizophreniform or schizoaffective disorder as per the Structured Clinical Interview for DSM-IV Disorders (“SC1D-FV”) (see, e.g., References 83 and 84); negative urine toxicology; stable, outpatient medication-free status for at toast three weeks. Exclusion criteria were: diagnosis of bipolar disorder, active substance use disorders (e.g., except tobacco use disorders) or current substance use based on urine toxicology. Patients were recruited from the outpatient research facilities at NYSP1. Psychosis severity was measured with the positive subscale of the Positive and Negative Syndrome Scale (“PANSS”; positive total score can be referred to as PANSS-PT) (see, e.g., Reference 85); PANSS measures of negative symptoms and general psychopathology (e.g., PANSS-NT and PANSS-GT, respectively) were used as control variables.

Exemplary CHR sample. CHR individuals were recruited from a longitudinal cohort study at the Center of Prevention and Evaluation (“COPE”) at NYSPI. COPE offers treatment to English-speaking individuals, aged 14 to 30 years, who are deemed to be at high-risk for psychosis. These CHR individuals were help-seeking and met criteria for at least one of three psychosis-risk syndromes. as assessed with the Structured Interview for Prodromal Syndromes (“SIPS”). (See, e.g., Reference 86). This instrument was also used to measure the severity of attenuated positive psychotic symptoms (“SIPS-PT”); SIPS measures of negative symptoms (“SIPS-NT”) and general symptoms (“SIPS-GT”) were used as control variables.

Although the exemplary system, method. and computer-accessible medium was used to assess correlates of psychosis severity within the clinical psychosis groups, two separate, non-overlapping, groups of healthy controls were used for exploratory comparison purposes: one (e.g., n=30) age-matched to the schizophrenia group and another (e.g., n=15) age-matched to the CHR group. These groups were recruited through advertisements and word of mouth. Healthy controls were excluded for: current or past axis I disorder (e.g., except tobacco use disorder; as per the SCID-IV), history of neurological disorders or current major medical illness, and first degree relatives with a history of psychotic disorder.

Table 2 above shows demographic and clinical information for all relevant groups (e.g. information On psychosis controls is shown in Table 4 below). Socio-economic status was measured with the Hollingshead interview. (See, e.g., Reference 8.7).

TABLE 3
Sociodemographic and Positron Emission
Tomography (PET) data for PET study sample

| | | |
|---|---|---|
| | | Patients with |
| | Healthy controls | schizophrenia |
| Characteristic | (n = 9) | (n = 9) |

Sociodemographic characeristics

| Sex (male) | | 6 (66%) | 6 (66%) |
|---|---|---|---|
| Age, years [range] | | 31.9 ± 2.5 | 31.9 ± 3.2 |
| | | [23-59] | [20-47] |
| Race/ | African- | 4 | 5 |
| ethnicity | American | | |
| | Asian | 1 | 1 |
| | Caucasian | 2 | 2 |
| | Hispanic | 2 | 1 |

| | | |
|---|---|---|
| Subject SES | 45.1 ± 5.1 | 20.7 ± 2.8 |
| Parental SES | 47.9 ± 2.0 | 36.3 ± 43 |
| Tobacco Users | 2 (22%) | 3 (33%) |
| Antipsychotic history | — | 5/4 |
| (drug naïve/drug free¹) | | |

PET parameters

| | | |
|---|---|---|
| Hours post-amphetamine² | 5.3 ± 1.3 | 5.3 ± 1.0 |
| Amphetamine level (ng/mL)² | 74.1 ± 13.3 | 70.1 ± 10.4 |
| Radiotracer Mass (μg)³ | 2.3 ± 2.1, 2.5 ± 1.3 | 1.8 ± 0.6, 2.0 ± 0.9 |
| Radiotracer Dose (mCi)³ | 8.4 ± 2.8, 11.2 ± 2.6 | 10.0 ± 3.1, 10.8 ± 2.9 |

Means ± standard error are given for continuous variables; number (and percentage) are given for categorical variables. P-values for group comparisons are given based on two sample t-tests for continuous variables and X² tests for categorical variables.
¹Antipsychotic medication status was considered “drug naïve” if lifetime exposure <6 weeks and none in as 3 weeks, and “drug-free” if one in past 3 weeks,
²Mean at start of scan 5-7 hours post-amphetamine,
³Mean for scans at baseline and 5-7 hours post amphetamine.
SES: socioeconomic status.

TABLE 4
Characteristics of psychosis sample and specific healthy-control groups

| | | | | | | |
|---|---|---|---|---|---|---|
| | | | Clinical | Controls | | |
| | | Controls for | high-risk | for | Schizophrenia | CHR vs. |
| | Schizophrenia | schizophrenia | individuals | CHR | vs. controls | controls |
| Characteristic | (n = 33) | (n = 30) | (n = 25) | (n = 15) | (p-value) | (p-value) |

Socio-demographic characteristics

| Age, years | | 33.9 ± 2.2 | 34 ± 2.2 | 25.6 ± 0.97 | 23.5 ± 0.86 | 0.97 | 0.15 |
|---|---|---|---|---|---|---|---|
| Sex (male) | | 23 (70%) | 18 (60%) | 13 (52%) | 9 (60%) | 0.42 | 0.46 |
| Race/ | African- | 17 (52%) | 12 (40%) | 6 (24%) | 3 (20%) | 0.71 | 0.28 |
| ethnicity | American | | | | | | |
| | Asian | 2 (6%) | 1 (3%) | 1 (4%) | 2 (13%) | | |
| | Caucasian | 7 (21%) | 6 (20%) | 10 (40%) | 3 (20%) | | |
| | Hispanic | 5 (15%) | 7 (23%) | 2 (8%) | 4 (27%) | | |
| | Mixed | 2 (6%) | 4 (13%) | 6 (24%) | 2 (13%) | | |

| | | | | | | |
|---|---|---|---|---|---|---|
| Parental SES | 41.7 ± 2.4 | 41.9 ± 2.2 | 44.5 ± 2.2 | 56.9 ± 3.8 | 0.95 | 0.008 |

Clinical Characteristics

| Nicotine users | | 3 (11%) | 3 (12%) | 1 (5%) | 0 (0%) | 0.96 | 1 |
|---|---|---|---|---|---|---|---|
| PANSS | Positive total | 16.9 ± 0.93 | 7.1 ± 0.09 | — | — | <0.0001 | — |
| | [range, 7-49] | | | | | | |
| | Negative total | 15.4 ± 0.99 | 8.9 ± 0.5 | — | — | <0.0001 | — |
| | [range, 7-49] | | | | | | |
| | General total | 32.8 ± 1.8 | 16.7 ± 0.39 | — | — | <0.0001 | — |
| | [range, 16-112] | | | | | | |
| SIPS | Positive total | — | — | 15.6 ± 0.70 | 2.8 ± 0.65 | — | <0.0001 |
| | [range, 0-30] | | | | | | |
| | Negative total | — | — | 17.4 ± 0.91 | 2.7 ± 1.02 | — | <0.0001 |
| | [range, 0-36] | | | | | | |
| | Disorganization | — | — | 11.2 ± 0.53 | 1.5 ± 0.35 | — | <0.0001 |
| | total | | | | | | |
| | [range, 0-24] | | | | | | |
| | General total | — | — | 13.7 ± 0.68 | 2 ± 0.53 | — | <0.0001 |
| | [range, 0-24] | | | | | | |

Means ± standard error are given for continuous variables; number (and percentage) are given for categorical variables.
P-values for group comparisons are given based on two-sample t-tests for continuous variables and X² tests for categorical variables.
SES; socio-economic status.
PANSS: Positive and Negative Syndrome Scale (positive or psychotic symptoms of schizophrenia include hallucinations and delusions; negative symptoms include emotional withdrawal and amotivation).
SIPS: Structured Interview for Prodromal Syndromes.

### Exemplary Supplemental Results

### Exemplary Topographic Relationships to Dopamine Release Capacity and Psychosis Within SN Voxels

SN voxels with a stronger relationship between NM-MRI CNR and dopamine release capacity tended to be more lateral and anterior, with no clear gradient along the superior-inferior axis (βₓ=0.015, t₁₃₃₇=5.87, p=10⁻⁸; β_{y}=0.036, t₁₃₃₇ =17.1, p=10⁻⁵⁹; β_{z}=0.001, t₁₃₃₇=−0.30. p−0.76; multiple-linear regression analysis predicting partial r across SN voxels as a function of their coordinates in x [absolute distance from the midline], y, and z directions).

Psychosis-overlap voxels, in turn, tended to predominate in ventral and anterior aspects of the SN, and to a lesser extent, in lateral aspects of the. SN (βₓ=0.22, t₁₈₀₃=2.86, p=0.004; β_{y}=0.45; t₁₈₀₃=6.14, p=10⁻⁹; β_{z}=−0.65, t₁₈₀₃=−6.69, p=10⁻¹¹; logistic regression analysis predicting presence of psychosis-overlap voxels as a function of their coordinates in x [absolute distance from the midline], y, and z directions).

### Exemplary Voxelwise Analyses Relating NM-MRI CNR to Psychosis Severity in Each Clinical Group

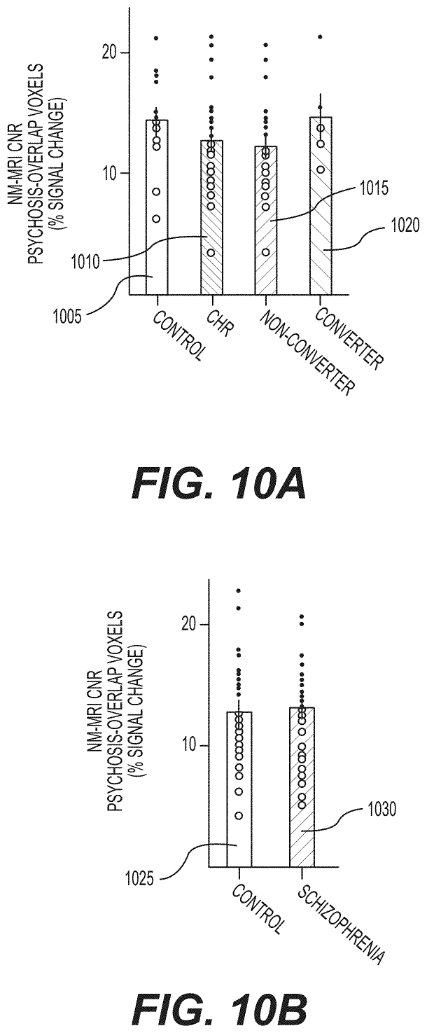

When analyzing each clinical group separately, it was determined that higher CNR in the SN correlated significantly with more severe psychosis in schizophrenia (e.g., PANSS-PT scores: 404 of 1807 SN voxels; p_{corrected}=0.007, permutation test; peak voxel MNI coordinates [x, y, z]: 5, −22, −20 mm) and non-significantly with attenuated psychosis in CHR individuals (e.g., SIPS-PT scores: 116 voxels, p_{corrected}=0.26, permutation test; peak voxel MNI coordinates [x, y, z]: 10, −25, −18 mm; and FIGS. 10A and 10B). Eliminating the CHR individual who was an outlier in the relationship of CNR in psychosis conjunction voxels to SIPS-PT (see, e.g., FIGS. 10A and 10B) increased the number of voxels where higher CNR correlated to SIPS-PT, although this relationship did not reach statistical significance in the permutation test correcting for multiple comparisons (e.g., 189 voxels, p_{corrected}=0.18).

FIG. 10A shows an exemplary graph illustrating a comparison of clinical high-risk individuals fur psychosis to age-matched healthy controls (e.g., bar 1005) according to an exemplary embodiment of the present disclosure. All CHR individuals are shown (e.g., bar 1010) as well as the subgroups of CHR individuals who did and did not subsequently convert to full-blown psychotic illness (e.g., bars 1015 and 1020). FIG. 10B shows an exemplary graph illustrating a comparison of unmedicated patients (e.g., control bar 1025) with schizophrenia (e.g., bar 1030) to age-matched healthy controls according to an exemplary embodiment of the present disclosure. Error bars indicate means and SEM. Individual data points represent subjects. No statistically significant differences in NM-MRI CNR extracted from psychosis-overlap voxels were seen for any group comparisons. Note that this is not representative of all SN voxels or voxels that may have shown trends for group differences (e.g., but that did not survive the corrected threshold). The lack of group differences in psychosis-overlap voxels indicates that SN regions that exhibit psychosis effects do not exhibit diagnosis effects

### Exemplary Comparison of MN-MRI CNR Across Groups

Aged-matched healthy control groups (e.g., n=30 and n=15, respectively) did not significantly differ from patients with schizophrenia (e.g., n=33) or CHR individuals (e.g., n=25) in CNR of the psychosis-overlap voxels, although, numerically, the mean CNR was higher in schizophrenia than in age-matched controls and was higher in CHR individuals who went on to develop schizophrenia compared to those CHR individuals who did not and age-matched controls. (See, e.g., graphs shown in FIGS. 10A and 10B),

The exemplary voxel based analysis procedure based on the dopamine biomarker neuromelanin can be used to detect dopamine based psychosis in patients with schizophrenia. There are currently no approved imaging tests that are able to diagnose mental illness, differentiate between different mental illnesses, predict the course of mental illness, predict future response to treatment, or predict future conversion to mental illness in high risk individuals. The exemplary system, method, and. computer-accessible medium can be performed with a standard hospital MRI machine. The exemplary voxel-based procedure, when method applied to NM-MRI, can be used as a biomarker of dopamine based psychosis in patients with schizophrenia the clinical setting. The exemplary system, method, and computer-accessible medium can also be used to predict conversion to schizophrenia in people who are at high risk. Additionally, the exemplary system, method, and computer-accessible medium can be used to diagnose or predict the development of Parkinson's disease, dementia with lewy bodies, multiple system atrophy, progressive supranuclear palsy, corticobasal degeneration, and parkinsonism-dementia complex of Guam.

### Resonance Imaging Protocols for Region-of-Interest and Voxelwise Analyses

The exemplary effect of various acquisition and preprocessing parameters on the strength can be analyzed. and. a test-retest reliability of the NM-MRI signal can be evaluated to determine optimized protocols for both ROI and voxelwise measures. Three new NM-MRI sequences with slice-thickness of 1.5 mm, 2 mm, and 3 mm were compared to the literature standard sequence with 3 mm slice-thickness. (See, e.g., References 978 and 99). Using the exemplary acquisition protocol, across the two acquired scans, ICC values were obtained indicating excellent reliability and high CNR, which could be achieved with a different set of parameters depending on the measures of interest and experimental constraints such as acquisition time. A detailed analysis of the CNR and ICC provide evidence for the optimal spatial-normalization software, number of measurements (acquisition time), slice thickness, and spatial smoothing.

### Exemplary Methods

Ten (10) healthy subjects underwent 2 MRI exams (e.g., test and re-test) on a 3T Prisma MRI (Siemens, Erlangen, Germany) using a 64-channel head coil. The test-retest scans were separated by a minimum of 2 days. inclusion criteria were: age between 18 and 65 years and no MRI contraindications. Exclusion criteria were: history of neurological or psychiatric diseases, pregnancy or nursing, and inability to provide written consent.

### Exemplary Magnetic Resonance Imaging

A T1-weighted (“T1w”) image was acquired for processing of the NM-MRI image using a 3D magnetization prepared rapid acquisition gradient echo (“MPRAGE”) sequence with the following parameters: spatial resolution=0.8×0.8×0.8 min³; field-of-view (FOV)=166×240×256 mm³; echo time (TE)=2.24 ms; repetition time (TR)=2,400 ms; inversion time (TI)=1060 ms; flip angle=8°; in-plane acceleration, GRAPPA=2, (see, e.g., Reference 109); bandwidth=210 Hz/pixel. A T2-weighted (“T2w”) image was acquired for processing of the NM-MRI image using a 3D sampling perfection with application-optimized contrasts by using flip angle evolution (“SPACE”) sequence with the following parameters: spatial resolution=0.8×0.8×0.8 mm³; FOV=166×240×256 mm³; TE=564 ms; TR=3,200 ms; echo spacing=3.86 ms; echo train duration=1,166 ms; variable flip angle (e.g, T2 var mode); in-plane acceleration=2; bandwidth=744 Hz/pixel. NM-MRI images were acquired using 4 different gradient 2D recalled echo sequences with magnetization transfer contrast (e.g., 2D GRE-MTC). (See, e.g., Reference 99). The following parameters were consistent across the 4 2D GRE-MT sequences: in-plane resolution=0.4×0.4 mm²; FOV=165×220 mm²; flip angle=40°; slice gap=10 mm; bandwidth=390 Hz/pixel; MT frequency offset=1.2 kHz; MT pulse duration=10 ms; MT flip angle=300°; partial k-space coverage of MT pulse. (See, e.g., Reference 99). The partial k-space coverage MT pulses were applied in a trapezoidal fashion, (see, e.g., Reference 129), with ramp-up and ramp-down coverage of 20% and plateau coverage of 40%. Other 2D GRE-MTC sequence parameters that differed across the 4 sequences are listed in Table 5. The order of the 4 NM-MRI sequences was randomized across all subjects and sessions.

TABLE 5
2D GRE-MTC Sequence Parameters Used for NM-MRI.

| | | | Slice- | | Number | Acquisition | |
|---|---|---|---|---|---|---|---|
| | TE | TR | Thickness | Number | of | Time | In-Plane |
| Sequence | (ms) | (ms) | (mm) | of Slices | Acquisitions | (minutes:s) | Acceleration |
| NM-1.5 mm | 4.11 | 444 | 1.5 | 16 | 11 | 10:51 | 2 |
| NM-2 mm | 3.61 | 321 | 2 | 12 | 15 | 10:42 | 2 |
| NM-3 mm | 3.61 | 214 | 3 | 8 | 23 | 10:56 | 2 |
| NM-3 mm | 3.87 | 273 | 3 | 10 | 10 | 11:02 | None |
| Standard | | | | | | | |

### Exemplary Neuromelanin-MRI Placement Protocol

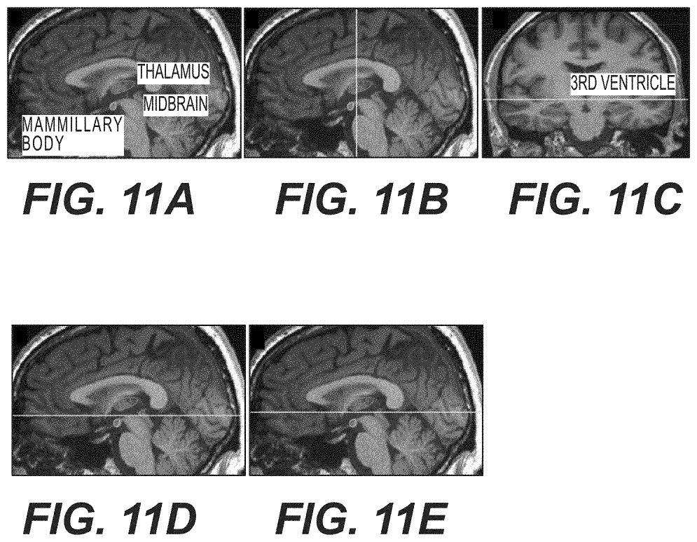

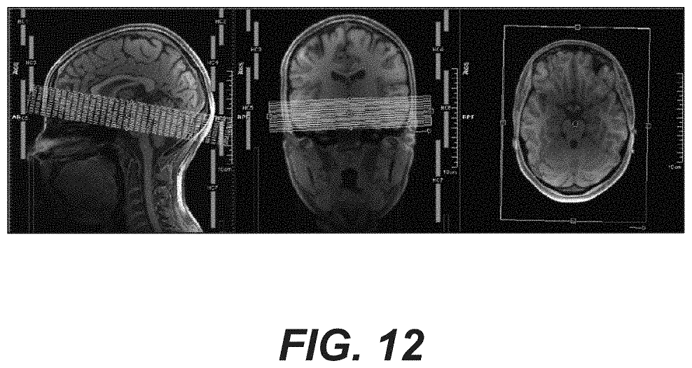

Given the limited coverage of the NM-MRI protocol in the inferior-superior direction (e.g., approximately 30 mm), a detailed NM-MRI volume placement procedure based on distinct anatomical landmarks was developed to improve within-subject and across-subject repeatability. The placement protocol makes use of the sagittal, coronal, and axial 3D T1w images. Furthermore, the coronal and axial images were reformatted along the anterior commissure-posterior commissure (“AC-PC”) line. The following is an exemplary procedure used for NM-MRI volume placement:
1. Identification of the sagittal image showing the greatest separation between the midbrain and thalamus. (See image shown in FIG. 11A).
2. Using the sagittal image from the end of Procedure 1, finding the coronal plane that identities the most anterior aspect of the midbrain. (See image shown in FIG. 11B).
3. Using the coronal image from the end of Procedure 2, finding the axial plane that identifies the inferior aspect of the third ventricle. (See images shown in FIGS. 11C and 11D).
4. Setting the superior boundary of the NM-MRI volume to be about 3 mm (e.g., within about plus or minus 20% of 3 mm) superior to the axial plane from the end of Procedure 3. (See image shown in FIG. 11E).
   An example of the final NM-MRI volume placement from a representative subject is shown in the exemplary images in FIG. 12.

### Exemplary Neuromelanin-MRI Preprocessing

The intra-sequence acquisitions were realigned to the first acquisition to correct for inter-acquisition motion. The motion corrected NM-MRI images were subsequently averaged. The average NM-MRI images were then coregistered to the T1w image. The T1w image was spatially normalized to a standard MNI template using 4 different software: (i) ANTs, (see, e.g., References 95, and 96), (ii) FSL, (see, e.g., References 92 and 115), (iii) SPM12's Unified Segmentation (e.g., referred to as SPM12 throughout), (see, e.g., References 94 and 131), and (iv) SPM12's DARTEL (e.g., referred to as DARTEL throughout). (See, e.g., References 93 and 131). The warping parameters to normalize the T1w image to the MNI template were then applied to the coregistered NM-MRI images using the respective software. The exemplary resampled resolution of the spatially normalized NM-MRI images was 1 mm, isotropic. Additionally, the spatially normalized NM-MRI images were spatially smoothed using 3D Gaussian kernels with full-width-at-half-maximum (“FWHM”) of 0 mm (e.g., no smoothing), 1 mm, 2 mm, and 3 mm. All analyses using manually traced ROIs used the standard 1 mm spatial smoothing. All ROI-analysis results used 0 mm of spatial smoothing and all voxelwise-analysis results used 1 mm of spatial smoothing unless otherwise specified.

### Exemplary Neuromelanin-MRI Analysis

The NM-MRI CNR at each voxel V was calculated as the relative change in NM-MRI signal intensity I from a reference region RR of white matter tracts known to have minimal NM content, the crus cerebri (“CC”), (see, e.g., Reference 97), and CNR_{V}=[I−mode(I_{RR})]/mode(I_{RR}).

Two-way mixed, single score ICC [ICC(3,1)], (see, e.g., Reference 141), were used to assess the test-retest reliability of NM-MRI. This ICC is a measure of consistency between the first and second measurements that does not penalize consistent changes across all subjects (e.g., if the retest CNR can be consistently higher than the test CNR for all subjects). The maximum ICC was 1. indicating perfect reliability, ICC over 0.75 indicates “excellent” reliability, ICC between 0.75 and 0.6 indicates “good” reliability, ICC between 0.6 and 0.4 indicates “fair” reliability, and ICC under 0.4 indicates “poor” reliability. (See, e.g., Reference 100). ICC(3,1) values were calculated for three conditions: the average CNR within a given ROI (e.g., ICC value per ROI; ICC_{ROI}); the across-subject voxelwise CNR (e.g., 1 ICC value per voxel; ICC_{ASV}); the within-subject voxelwise CNR (e.g., 1 ICC value per subject; ICC_{WSV}). ICC_{ROI} provides a measure of the reliability of the average CNR within an ROI across all subjects, thus providing a measure of the reliability of the ROI-analysis approach. ICC_{ASV} provides a measure of the reliability of CNR_{V} at each voxel within an ROI across all subjects, thus providing a measure of the reliability of the voxelwise-analysis approach. ICC_{WSV} provides a measure of the reliability of the spatial pattern of CNR_{V} across voxels within each of the subjects individually which provides a complementary measure of reliability of the voxelwise-analysis approach.

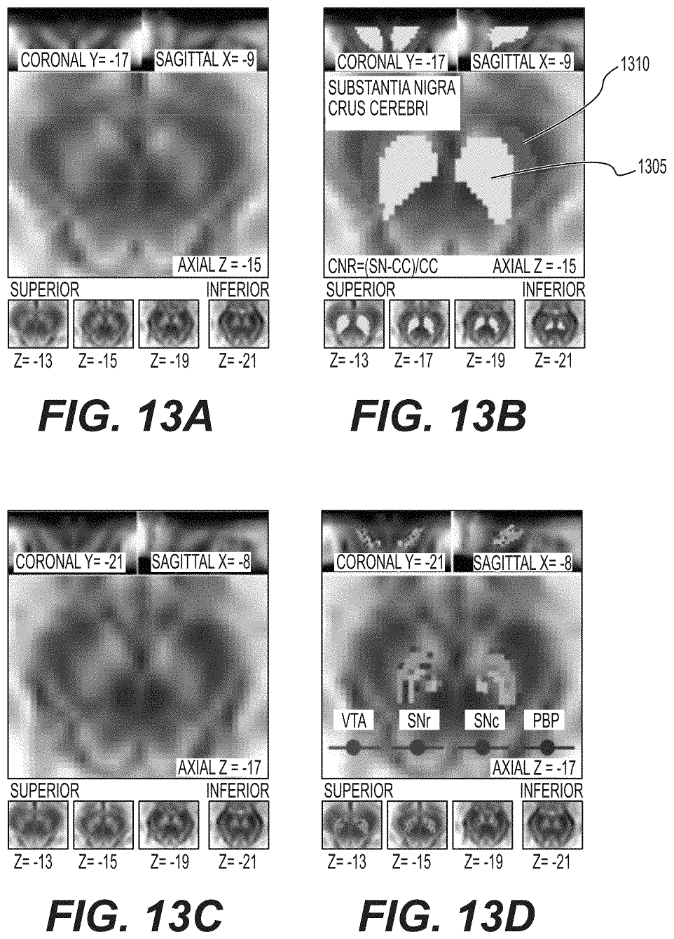

The ROIs used included a manually traced mask of the SN (see, e.g., Reference 97). and ROIs of the SN/VTA-complex nuclei: SN pars compacta (“SNc”). SN pars reticulata (“SNr”), ventral tegmental area (VTA), and parabrachial pigmented nucleus (“PBP”) as defined from a high-resolution probabilistic atlas. (See, e.g., Reference 130). FIGS. 13A-13D show the ROIs overlaid on a template NM image according to an exemplary embodiment of the present disclosure. In particular, FIG. 13A illustrates the average NM-MRI image created by averaging the spatially normalized NM-MRI images from 10 individuals in MNI space. Note the high signal intensity in the SN. FIG. 13B illustrates masks for the SN (e.g., voxels 1305) and the CC (e.g., voxels 1310) reference region (e.g., used in the calculation of CNR) are overlaid onto the template in FIG. 13A. These anatomical masks were made by manual tracing on a NM-MRI template from a previous study. FIG. 13C illustrates the same average NM-MRI image from FIG. 13A. FIG. 13D illustrates probabilistic masks for the VTA, SNr, SNc, and PBP as defined from a high-resolution probabilistic atlas overlaid onto the template in FIG. 13C. The scaling for probabilistic masks goes from P=0.5 to P=0.8.

### Exemplary Results

The test-retest MRI exams were separated by 13±13 (e.g., mean±standard deviation) days on average with a median of 8 days, minimum of 2 days, and maximum of 38 days. Of the 10 subjects, 4 were male and 6 were female; 4 were Caucasian and 6 were Asian; 9 were right-handed and 1 was left-handed. The average age was 27±5 years (e.g., mean±standard deviation). None of the subjects reported current cigarette smoking or recreational drug use.

### Exemplary Acquisition Time

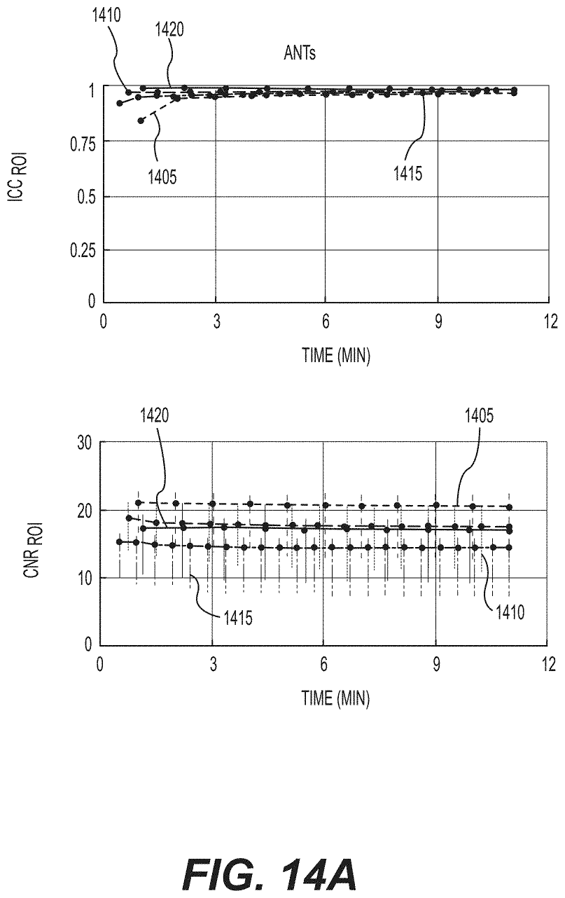

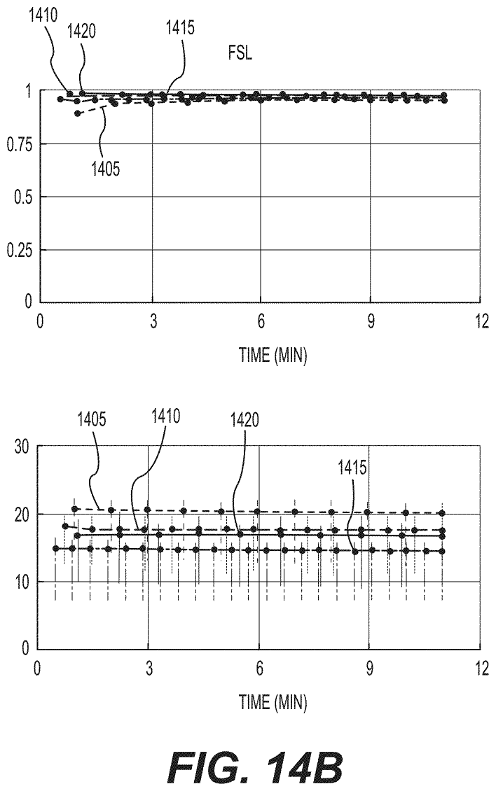

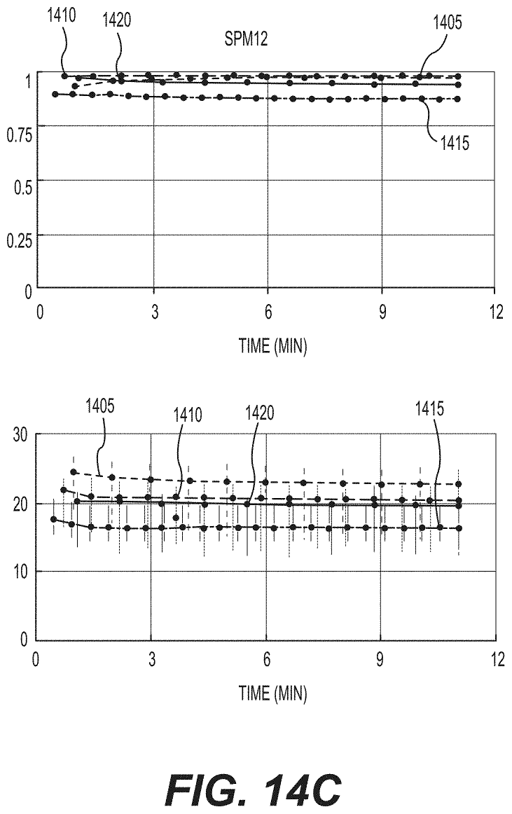

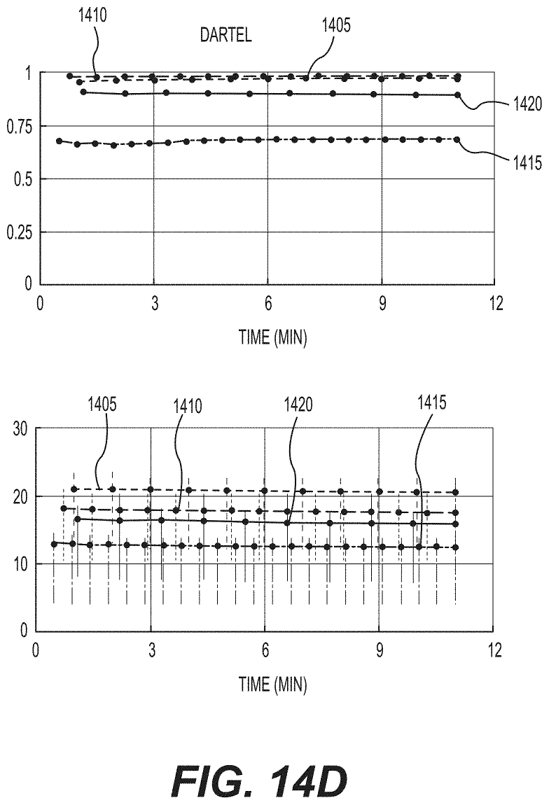

Plots of ICC_{ROI} and CNR_{ROI} within the manually traced mask as a function of acquisition time for each of the NM-MRI sequences and spatial normalization software are shown in the graphs of FIGS. 14A-14D, which illustrate NM-1.5 mm (e.g., line 1405), NM-2mm (e.g., line 1410), NM-3 mm (e.g., line 1415) and NM-3 mm standard (e.g., line 1420). For example, the top graph of each of FIGS. 14A-14C illustrates ICC_{ROI} and the bottom graph of each of FIGS. 14A-14C illustrates and CNR_{ROI} within the manually traced mask of the SN/VTA-complex (see e.g., FIG. 13B) as a function of acquisition time. Exemplary data points denote the median and error bars indicate the 25^{th} and 75^{th} percentiles. In general, all NM-MRI sequences and spatial normalization software achieved excellent test-retest reliability within 3 minutes of acquisition time and CNR_{ROI} was not affected by acquisition time. The NM-1.5 mm sequence had the highest CNR_{ROI} for all spatial normalization software while the NM-3 mm sequence had the lowest.

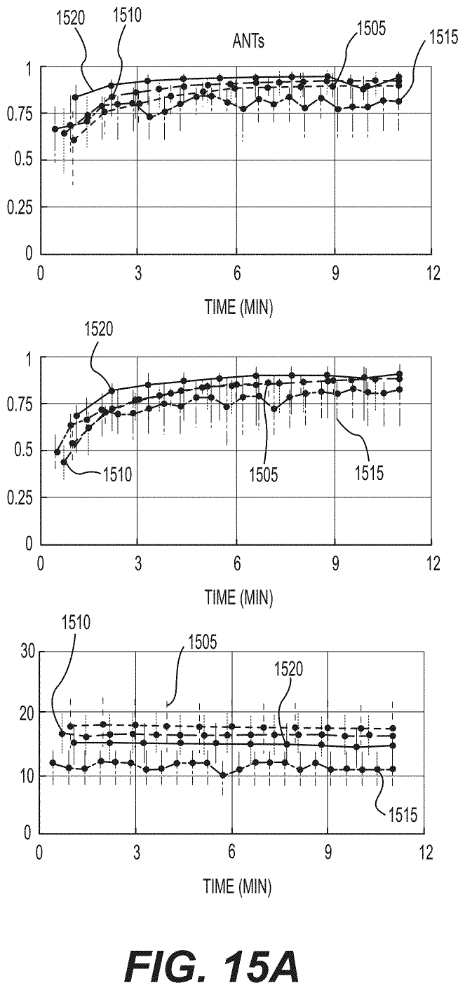

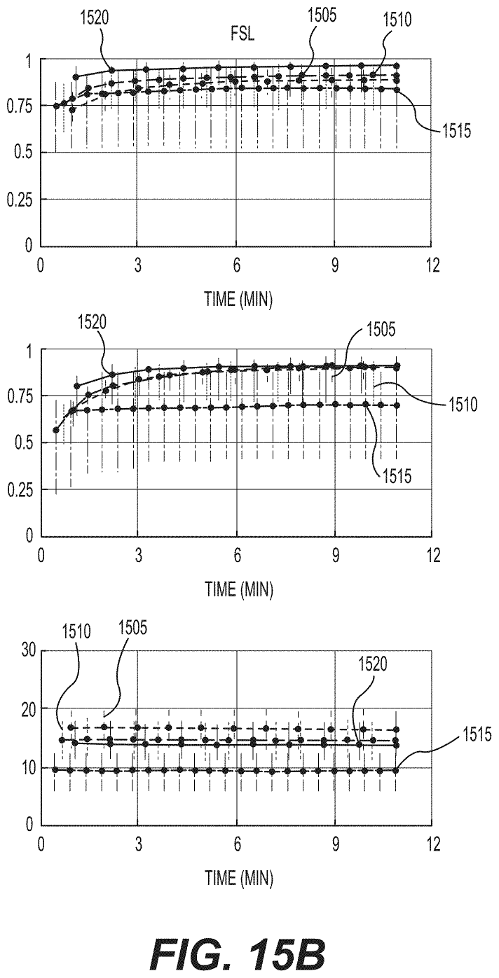

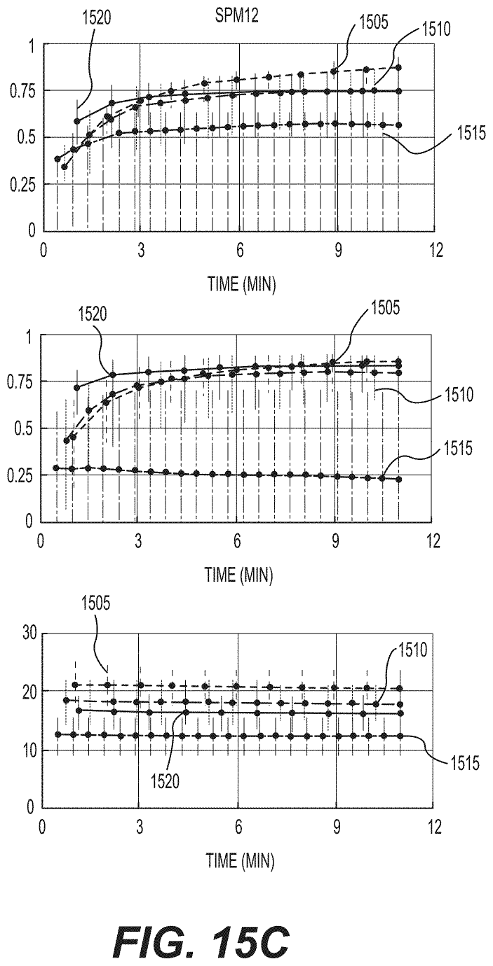

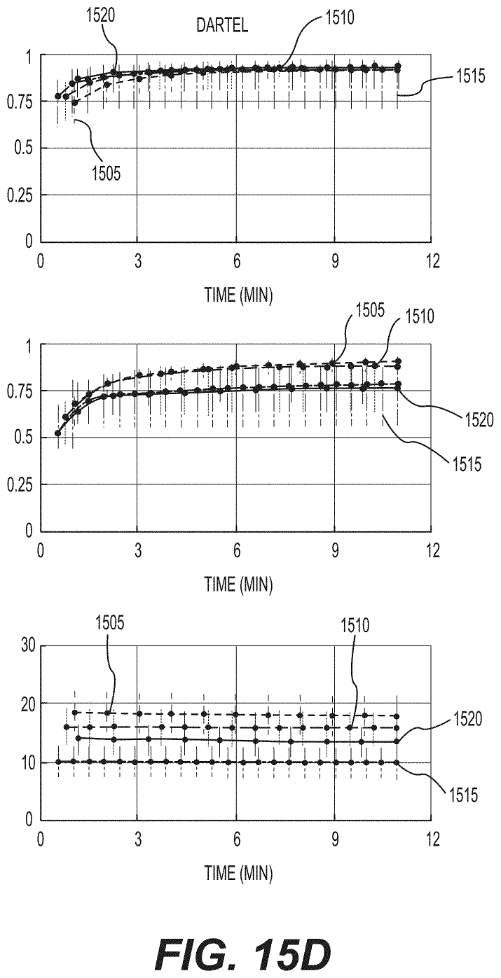

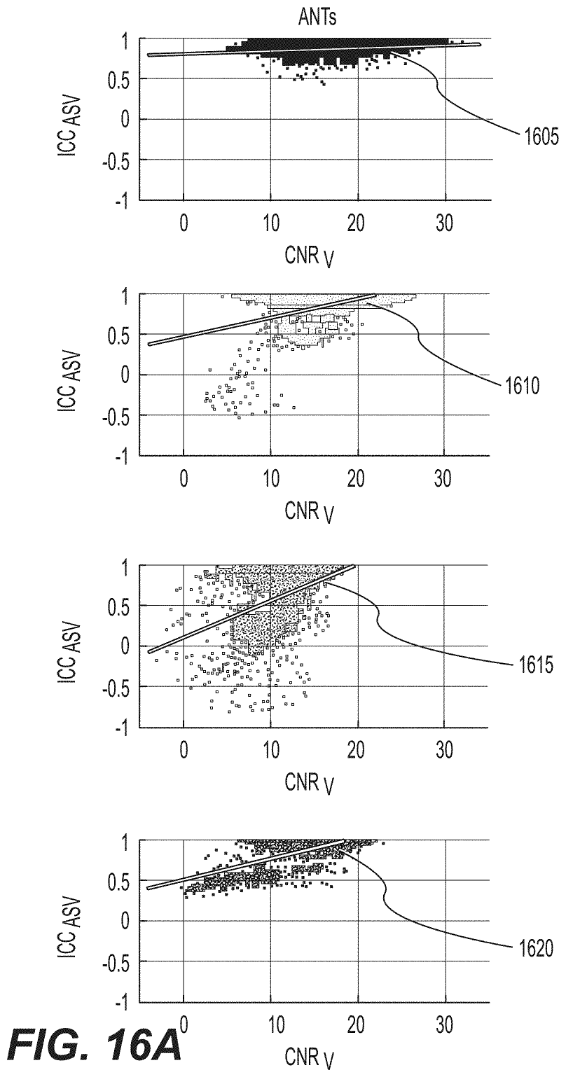

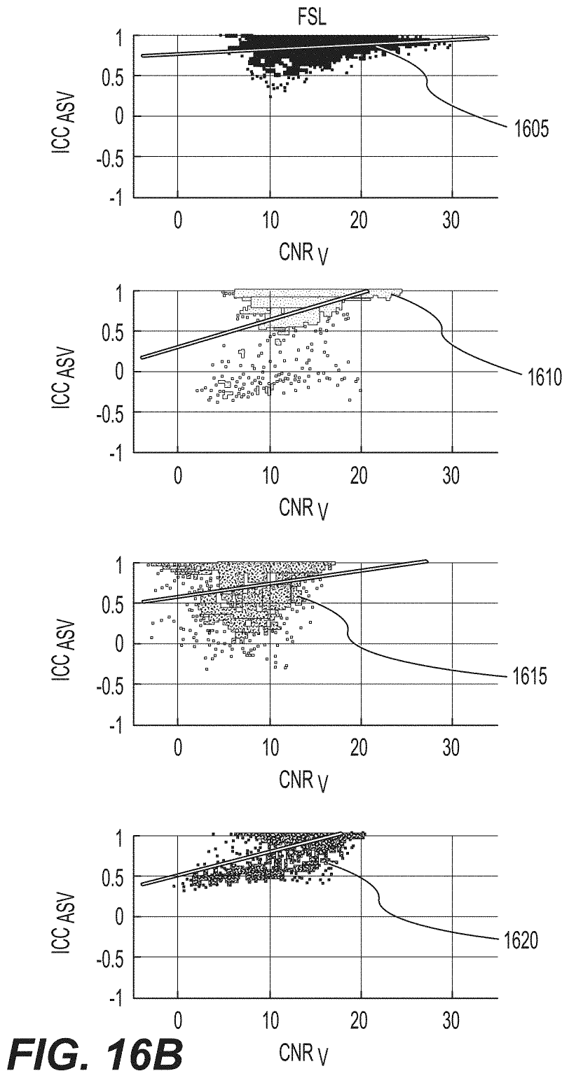

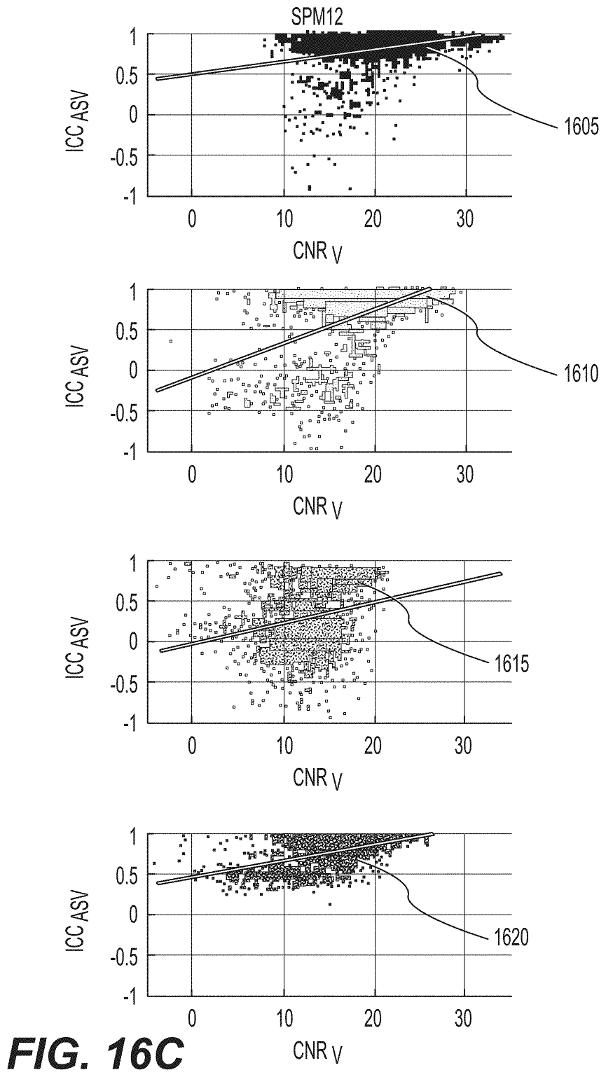

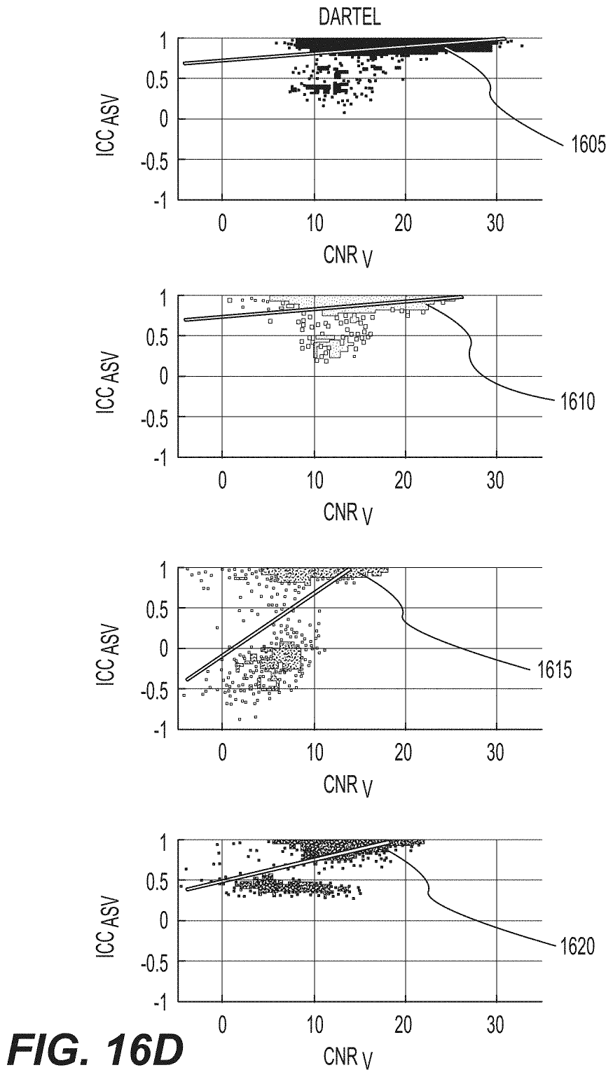

Plots of the ICC_{ASV}, TCC_{WSV}, and CNR_{V} within the manually traced mask as a function of acquisition time for each of the NM-MRI sequences and spatial normalization software are shown in the graphs of FIGS. 15A-15D, which illustrate NM-1.5 min (e.g., line 1505), NM-2 mm. (e.g., line 1510), NM-3 mm (e.g., line 1515) and NM-3 mm standard (e.g., line 1520). For example, the top graph illustrates ICC_{ASV}, the middle graph illustrates ICC_{WSV}, and the bottom graph illustrates CNR_{V} of voxels within the manually traced mask of the SN/VTA-complex (see e.g., FIG. 13B) as a function of acquisition time. Data points denote the median and error bars indicate the 25^{th} and 75^{th} percentiles. Spatial normalization software and NM-MRI sequences except for NM 3-mm achieved excellent test-retest reliability within 6 minutes of acquisition time and CNR_{ROI} was not affected by acquisition time. The NM-1.5 mm sequence had the highest CNR_{ROI} for all spatial normalization software while the NM-3 ram sequence had the lowest.

### Exemplary Choice of NM-MRI Sequence

Scatterplots of the ICC_{ASV} and CNR_{V} of each voxel within the manually traced mask for each of the NM-MRI sequences and. spatial normalization software are shown in the scatter plots shown in FIGS. 16A-16D, which illustrate NM-1.5 min (e.g., scatter plot 1605), NM-2 mm (e.g., scatter plot 1610), NM-3 mm (e.g., scatter plot 1615) and NM-3 mm standard (e.g., scatter plot 1620). Table 6 lists the 25^{th} percentile, median, and 75^{th} percentile of ICC_{ASV} and CNR_{V} values shown. in FIG. 16. The NM-1.5 mm sequence consistently showed. the highest CNR_{V}, greatest spread in CNR_{V}, lowest correlation between CNR_{V} and ICC_{ASV}, and high ICC_{ASV} across all spatial normalization software. Because the NM-1.5 mm sequence demonstrated the best performance, further optimization was performed for this sequence and the following sections only use data from this sequence.

TABLE 6
ICC_{ASV}, CNR_{V}, and Spearman’s rho of their relationship for each NM-MRI
sequence and spatial normalization software. ICC_{ASV} and CNR_{V} values are from within
the manually traced mask of the SN/VTA-complex (see, e,g., Figure 13B) and are listed
as 25^{th} percentile, median, 75^{th} percentile. Spearman’s rho values represent the
relationship between ICC_{ASV} and CNR_{V} of voxels within the manually traced mask.

| | Spatial | | | |
|---|---|---|---|---|
| | Normalization | | | |
| Sequence | Software | ICC_{ASV} | CNR_{V} | p |
| NM-1.5 mm | ANTs | 0.86, 0.91, 0.94 | 13.2, 17.3, 21.6 | 0.26 |
| | FSL | 0.82, 0.88, 0.92 | 12.6, 16.2, 19.7 | 0.22 |
| | SPM12 | 0.77, 0.86, 0.91 | 16.7, 20.4, 23.8 | 0.26 |
| | DARTEL | 0.85, 0.89, 0.92 | 15.3, 18.8, 22.3 | 0.25 |
| NM-2 mm | ANTs | 0.87, 0.92, 0.95 | 12.7, 16.1, 19.3 | 0.37 |
| | FSL | 0.73, 0.90, 0.95 | 11.5, 14.4, 17.5 | 0.49 |
| | SPM12 | 0.65, 0.81, 0.90 | 14.5, 17.9, 21.1 | 0.35 |
| | DARTEL | 0.81, 0.88, 0.92 | 14.3, 17.3, 19.9 | 0.26 |
| NM-3 mm | ANTs | 0.21, 0.77, 0.93 | 7.9, 10.6, 13.4 | 0.42 |
| | FSL | 0.51, 0.83, 0.94 | 5.9, 9.5, 12.2 | 0.33 |
| | SPM12 | −0.04, 0.24, 0.91 | 9.9, 12.4, 14.9 | 0.27 |
| | DARTEL | 0.32, 0.80, 0.86 | 7.3, 10.2, 12.9 | 0.54 |
| NM-3 mm | ANTs | 0.84, 0.95, 0.97 | 11.3, 14.6, 17.1 | 0.30 |
| Standard | FSL | 0.86, 0.94, 0.96 | 11.3, 13.7, 15.9 | 0.35 |
| | SPM12 | 0.70, 0.84, 0.89 | 13.2, 16.2, 18.6 | 0.42 |
| | DARTEL | 0.85, 0.91, 0.94 | 10.5, 13.2, 15.3 | 0.47 |

### Exemplary Choice of Spatial Normalization Software

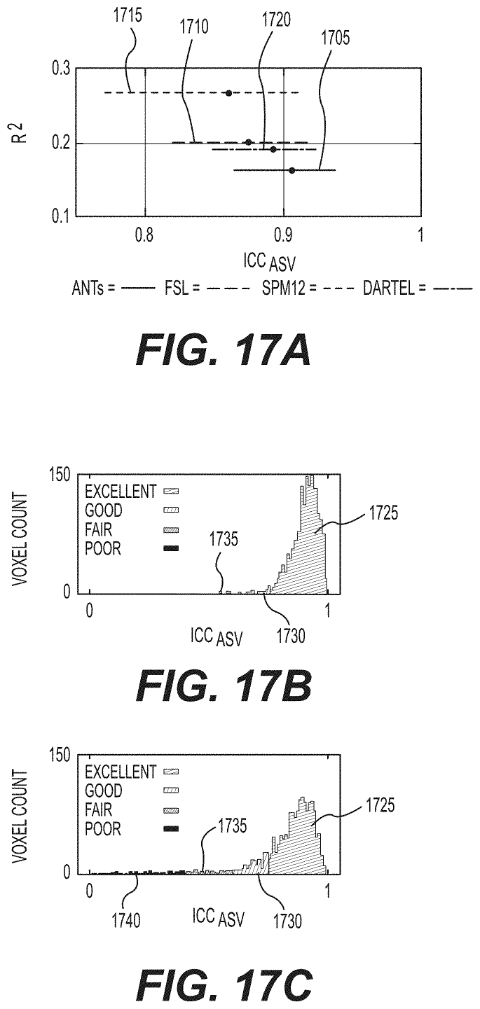

To determine which spatial normalization software can be used for voxel-wise-analysis of NM-MRI data, multiple linear regression analysis predicting ICC_{ASV} of voxels within the manually traced mask as a function of their coordinates in x (e.g., absolute distance from the midline), y, and z directions was used. With an optimal method, the ICC can be highest and homogeneous across voxels such that the voxel's anatomical location may not predict its associated ICC value. This analysis showed that ANTs achieved the best performance in that anatomical position was least predictive of ICC_{ASV} and it provided the highest ICC_{ASV}. FIG. 17A shows an exemplary graph of the predictive value (R²) of anatomical position on ICC_{ASV} and ICC_{ASV} of voxels within manually traced mask of the SNIVIA-complex (see e.g., FIG. 13B) for NM-1.5 mm sequence and each of the spatial normalization software according to an exemplary embodiment of the present disclosure.

In particular, FIG. 17A illustrates ANTₛ (e.g., line 1708), FSL (e.g., line 1710), SPM₁₂ (e.g., line 1715), and DARTEL (e.g., line 1720). Data points denote the median and error bars indicate the 25^{th} and 75^{th} percentiles. FIG. 178 shows an exemplary histogram of ICC_{ASV} of voxels within the manually traced mask for NM-1.5 mm sequence and ANTs spatial normalization software, which can be the best performing method., as shown in FIG. 17A according to an exemplary embodiment of the present disclosure. FIG. 17C shows an exemplary histogram of ICC_{ASV} of voxels within the manually traced mask for NM-1.5 mm sequence and SPM12 spatial normalization software, which can he the worst performing method as shown in FIG. 17.A. Area 1725 denotes excellent reliability (e.g., ICC over 0.75), area 1730 denotes good reliability (e.g., ICC between 0.75 and 0.6), area 1735 denotes fair reliability (e.g., ICC between 0.6 and 0.4), and area 1740 denotes poor reliability (e.g., ICC under 0.4). This result was consistent with a previous study where ANTs outperformed 13 other spatial-normalization procedures. (See, e.g., Reference 118).

### Exemplary Effect of Spatial Smoothing

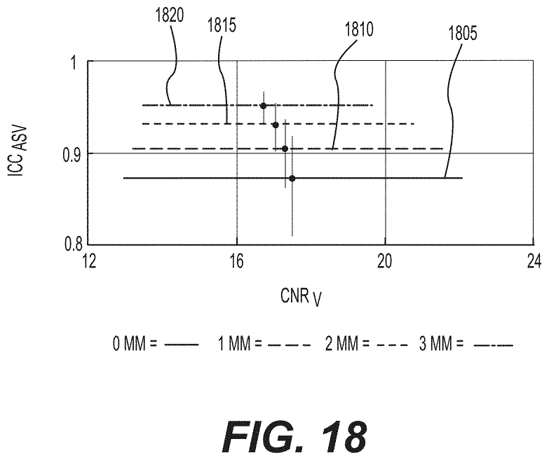

FIG. 18 shows an exemplary graph illustrating the effect of spatial smoothing on ICC_{ASV} and CNR_{V} of voxels within the manually traced mask of the SN/VTA-complex (see e.g., FIG. 13B) for different degrees of spatial smoothing tier 0 mm (e.g., line 1805), 1 mm (e.g., line 1810), 2 mm (e.g., line 1815) and 3 min (e.g., line 1820). Data points denote the median and error bars show the 25^{th} and 75^{th} percentiles. Greater amounts of spatial smoothing lead to significantly lower CNR_{V} and significantly higher ICC_{ASV} (Wilcoxon signed rank test, P<0.001 for all after correction for multiple comparisons). Relative to the one-lower degree of spatial smoothing (e.g., 2 mm vs 1 mm), spatial smoothing with 1 mm FSHM achieved the greatest increase in ICC_{ASV} and lowest decrease in CNR_{V}, 0.03 and −0.09, respectively. Although there was still a significant difference in ICC_{ASV} and CNR_{V} between spatial smoothing with FWHM of 0 mm and 1 mm, the minimal difference in CNR_{V} and overall improvement in the robustness of voxelwise-analysis and spatial normalization in particular, support the use of spatial smoothing with 1 mm FWHM.

Analyses Using Probabilistic Atlas of Dopaminergic Nuclei

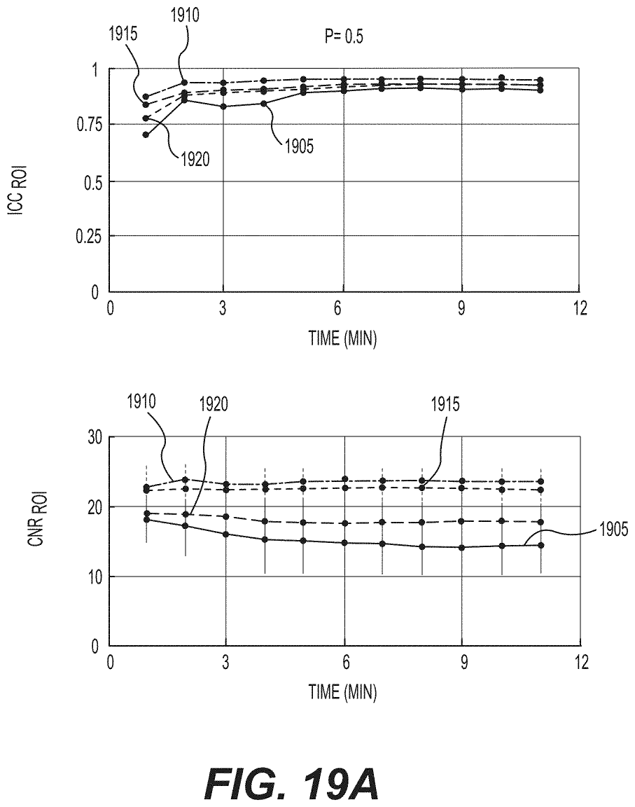

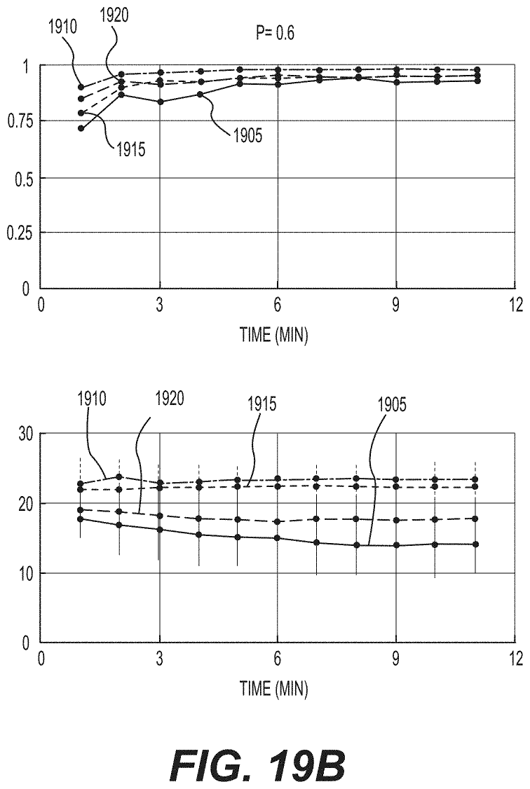

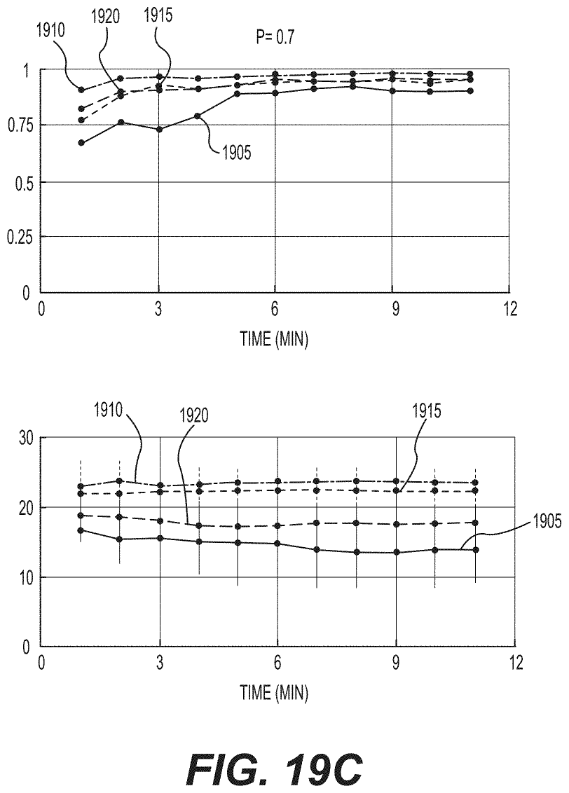

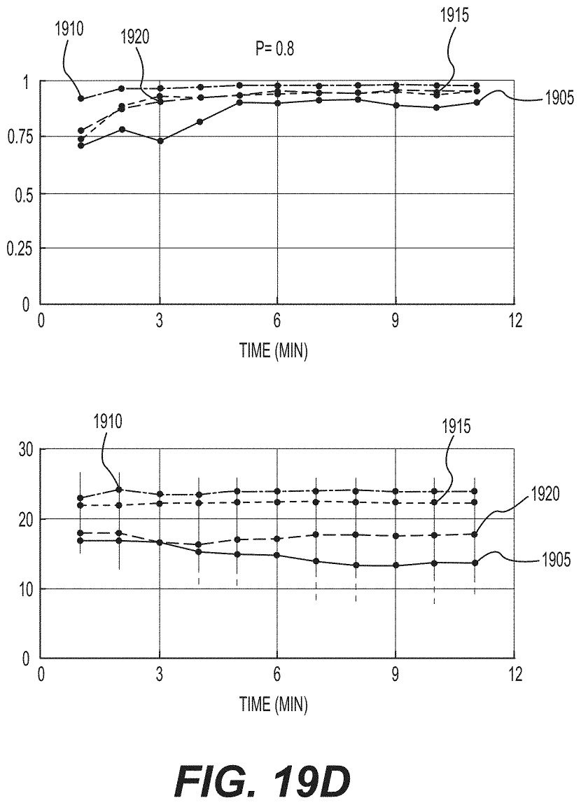

Using a recent high-resolution probabilistic atlas that identified the SN/VTA-complex nuclei, (see, e.g., Reference 130), the feasibility of obtaining reliable measures of NM-MRI signal in these nuclei were analyzed. Such measures can be valuable for basic and clinical neuroscience, particularly for the VTA. given its importance for reward learning, (see, e.g., References 125, 136 and 147), and affective processing. (See, e.g., References 104 and 108). Plots of the ICC_{ROI} and CNR_{ROI} within the probabilistic masks as a function of acquisition time for the NM-1.5 mm sequence and ANTs spatial normalization software and various probability cutoffs are shown in the graphs of FIGS. 19A-19D, which illustrate VTA (e.g., line 1905), SN_{R} (e.g., line 1910), SN_{C} (e.g., line 1915), and PBP line 1920). For example, the top graph of each of FIGS. 19A-19D illustrates ICC_{ROI} and the bottom graph illustrates CNR_{ROI} within the probabilistic masks of the SN/VTA-complex nuclei (see, e.g., FIG. 13D) with different probability cutoffs (0.5, 0,6, 0.7, and 0.8) as a function of acquisition time. Data points denote the median and error bars indicate the 25^{th} and 75^{th} percentiles

In general, excellent test-retest reliability was achieved for all nuclei and all probability cutoff values within 6 minutes of acquisition time. Similar to CNR in the manually traced mask, the CNR_{ROI} was not affected by acquisition time. The highest CNR was consistently observed in the SNr and SNc, then the PBP, and the lowest CNR in the VTA.

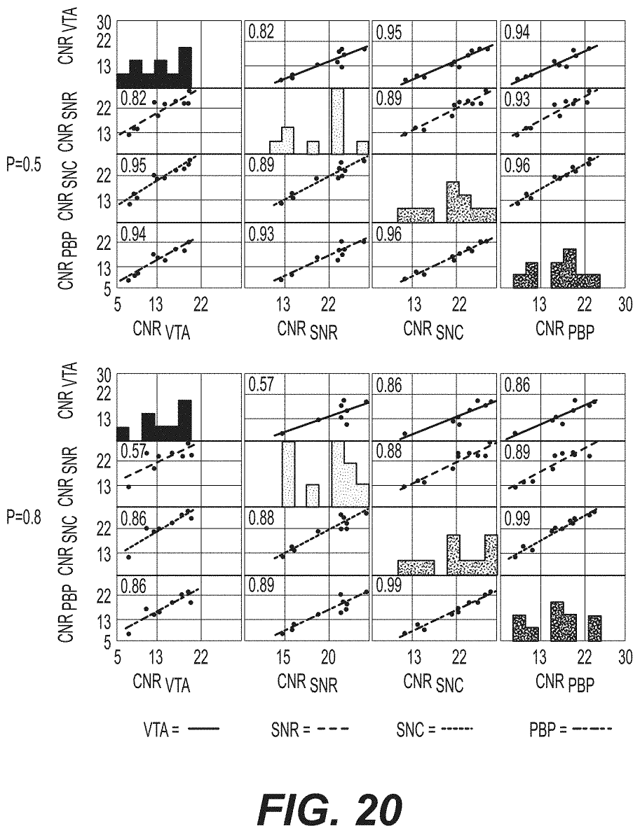

Having established the ability to reliably measure NM-MRI within SN/VTA-complex nuclei, how distinct the NM-MRI signal within each nuclei is was analyzed. The SN/VTA-complex nuclei are believed to have distinct anatomical projections and functional roles, so independently measuring signals from these nuclei can facilitate the investigation into these distinct anatomical circuits and functions, Although the nuclei are anatomically distinct, the potential for cross-contamination of the NM-MRI signal exists due to partial volume effects and spatial blurring due to MRI acquisition and the spatial normalization procedure. The independence of CNR_{ROI} values measured within the individual SN/VTA-complex nuclei was assessed by nonparametric Spearman's correlation, FIG. 20 shows a set of exemplary correlations and histograms of the CNR_{ROI} values within the 4 SN/VTA7-complex nuclei (see e.g., FIG. 13D) for the lowest (e.g., P=0.5) and highest (e.g., P=0.8) probability cutoffs. The value within each correlation plot is Spearman's rho. Overall, CNR was highly correlated across the four nuclei, particularly for ROI definitions based on a probability cutoff of P=0.5.

### Exemplary Discussion

The exemplary system, method and computer-accessible medium, according to an exemplary embodiment of the present disclosure, can utilize a volume placement protocol for NM-MRI in order to perform a test-retest study design to quantitatively derive recommendations for NM-MRI sequence parameters and preprocessing methods to achieve reproducible NM-MRI for ROI and voxelwise analyses. Additionally, by using a high-resolution probabilistic atlas, the reproducibility of NM-MRI measurements in specific nuclei within the SN/VTA-complex were determined. Overall, excellent reproducibility was observed in all ROIs investigated and for voxels within the ROIs. Based on the exemplary results, it can he beneficial to acquire at least 6 minutes of data for voxelwise-analysis or dopaminergic-nuclei-ROI-analysis and at least 3 minutes of data for standard ROI-analysis. Additionally, it can be beneficial to acquire NM-MRI data with 1.5 mm slice-thickness, using ANTs for spatial normalization, and performing spatial smoothing with a 1 mm FWHM 3D Gaussian kernel for voxelwise-analysis and no spatial smoothing for ROI-analysis, especially for analysis of nuclei.

High ICC values observed using the exemplary system, method and computer-accessible medium indicate that NM-MRI using 2D GRE-MT sequences achieve excellent reproducibility across several acquisition and preprocessing combinations. This can he in agreement with a previous report that observed an ICC_{ROI} value of 0.81 for SN in 11 healthy subjects. (See, e.g., Reference 121). Higher ICC_{ROI} values (e.g., approximately 0.92) were observed even though the. exemplary system, method and computer-accessible medium used a template defined SN mask and the two MRI scans were separated by 13±13 days instead of using a subject-specific semi-automated thresholding method for SN mask generation, (see, e.g., Reference 99). and having test-retest scans within a single session in one day (e.g., in which subjects were removed from the scanner after the first session, repositioned on the table, and scanned again). The exemplary system, method and computer-accessible medium, according to an exemplary embodiment of the present disclosure, can be used to identify anatomical landmarks to improve the reproducibility of volume placement across sessions. Previous studies focused on ROI measures, and did not measure voxel-wise ICC. In contrast, the exemplary system, method and computer-accessible medium can use voxel-wise CNR measures to obtain reliably. Another recent study measured ICC_{ASV} in 8 healthy and 8 patients with schizophrenia, also with both MRI sessions on the same day (approximately 1 hour apart). (See, e.g., Reference 97). That study observed a median ICC_{ASV} value of 0.64 and an ICC_{ROI} value of 0.96. The higher ICC_{ASV} observed in the present study could be due to the inclusion of only healthy subjects as well as the detailed volume placement protocol.

In addition to ICC values, the strength of the NM-MRI signal and the range of CNR values was used. Because correlation-based approaches can be common for voxel-wise-analysis, a greater range in CNR values within the SNNTA-complex can provide greater statistical power. Another important factor in the exemplary analysis was the relationship between CNR and ICC. To confirm that voxel-wise analysis effects are not driven solely by high, or low, CNR voxels due to lower measurement noise in those voxels, it can be beneficial to have homogeneously high ICC values independent of CNR. The exemplary ANTs-based procedure applied to NM-MRI data with 1.5 mm slice-thickness accomplishes this independence.

The exemplary system, method and computer-accessible medium, according to an exemplary embodiment of the present disclosure, was used to illustrate how the NM-MRI signal within the nuclei can be highly reproducible with approximately 6 minutes of data. Overall, the highest was observed CNR in the SNc and SNr, followed by the PBP, then the VTA. This can be consistent with reports of higher degree of NM pigmentation in the SN than the VTA. (See, e.g., References 112 and 123). However, the NM-MRI signal was highly correlated across nuclei. This finding can indicates that NM-MRI may only provide a measure of the general function of the dopamine system and may not be specific to nuclei with distinct anatomy and function. While this can be true, the exemplary study included a limited number of subjects. Additionally, it can be possible that the different functional domains of the dopamine system can be highly correlated in healthy subjects and small errors in the realignment and spatial normalization processes could cause signal from different nuclei becoming mixed. These concerns could be partially mitigated through the use of a voxelwise-analysis. (See, e.g., Reference 97).

The exemplary system, method and computer-accessible medium, according to an exemplary embodiment of the present disclosure, was used to test 2 NM-MRI sequences with 3 mm slice-thickness: NM-3 mm and NM-3 mm Standard. The main difference between these two NM-MRI sequences was the use of in-plane acceleration, the number of slices, the TE, and the TR. These parameters were changed relative to the literature standard protocol (e.g., NM-3 mm Standard) to accommodate the increased number of slices utilized for similar coverage in the higher resolution sequences (e.g, NM-1.5 mm and NM-2 mm). Although the higher resolution sequences did not seem to be affected, increased noise due to in-plane acceleration could have caused the lower reproducibility observed for the NM-3 mm sequence compared to the NM-3 mm Standard sequence. (See. e.g., Reference 132). An alternative explanation can be that the reduced number of slices results in reduced performance of the realignment and coregistration steps (resulting from less anatomical information for the procedures to work with) leading to reduced reproducibility. All images were manually inspected at each step and no obvious errors occurred but small-scale deviations in the preprocessing could impact the reproducibility.

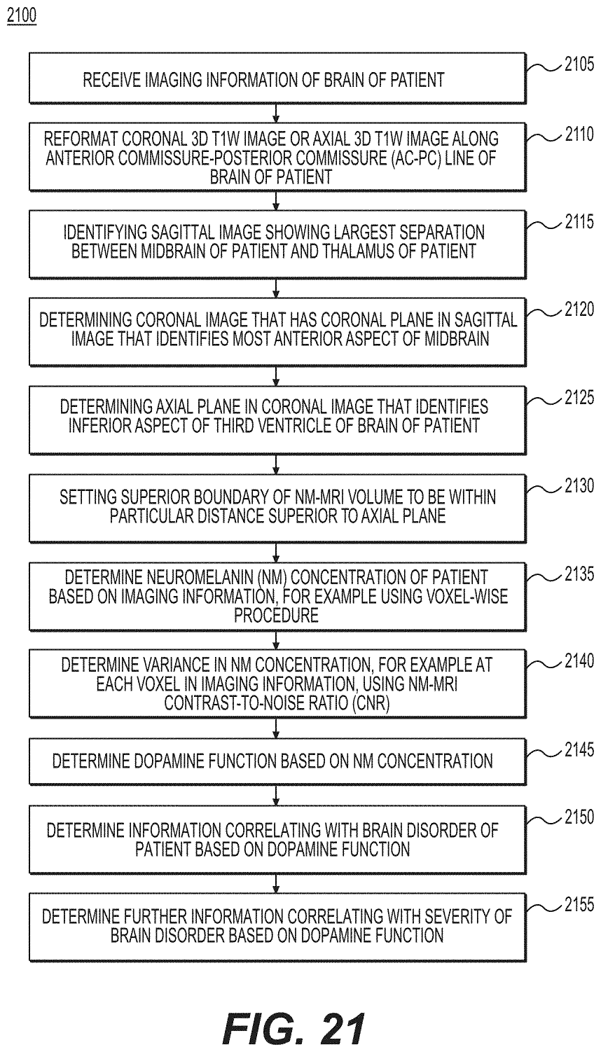

FIG. 21 shows a flow diagram of an exemplary method 2100 for determining a dopamine function of a patient according to an exemplary embodiment of the present disclosure. For example, at procedure 2105, imaging information of a brain of the patient can be received. At procedure 2110, a coronal 3D T1w image or an axial 3D T1w image can be reformatted along an anterior commissure-posterior commissure (AC-PC) line of the brain of the patient. At procedure 2115, a sagittal image showing a largest separation between a midbrain of the patient and a thalamus of the patient can be identified. At procedure 2120, a coronal image that has a coronal plane in the sagittal image that identifies a most anterior aspect of the midbrain can be determined. At procedure 2125, an axial plane in the coronal image that identifies an inferior aspect of a third ventricle of the brain of the patient can be determined. At procedure 2130, setting a superior boundary of the NM-MRI volume to be within a particular distance superior to the axial plane can be set. At procedure 2135, a Neuromelanin (NM) concentration of the patient can be determined based on the imaging information, for example using a voxel-wise procedure. At procedure 2140, a variance in the NM concentration, for example at each voxel in the imaging information, can be determined using a NM-MRI contrast-to-noise ratio (CNR). At procedure 2145, the dopamine function can be determined based on the NM concentration. At procedure 2150, information correlating with a brain disorder of the patient can be determined based on the dopamine function. At procedure 2155, further information correlating with a severity of the brain disorder can be determined based on the dopamine function.

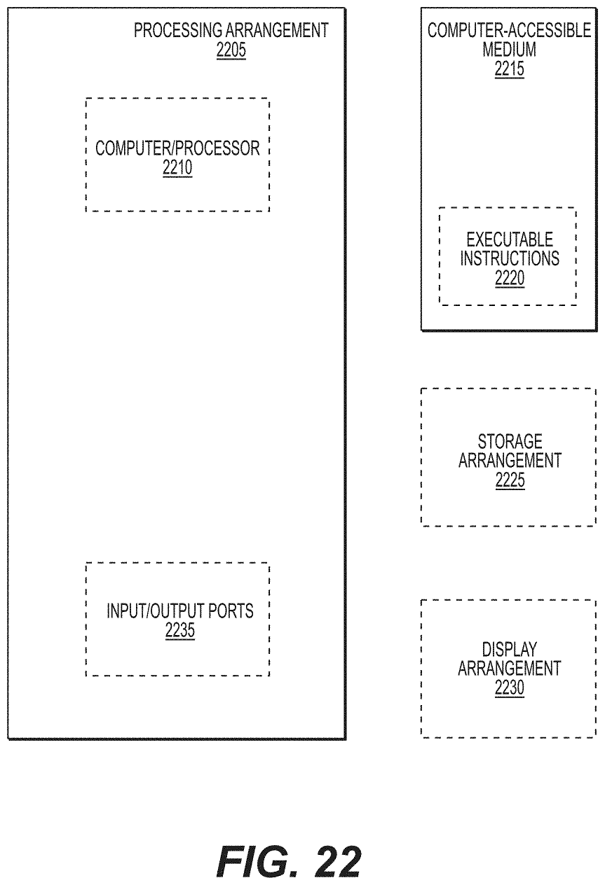

FIG. 22 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 2205. Such processing computing arrangement 2205 can be, for example entirely or a part of or include, but not limited. to, a computer/processor 2210 that can include, for example one or more microprocessors, and use instructions stored on it computer-accessible medium (e.g., RAM, ROM, hard drive, or other storage device).

As shown in FIG. 22, for example a computer-accessible medium 2215 (e.g., as described herein above, a storage device such as a hard disk, floppy disk, memory stick, CD-ROM, RAM, ROM, etc., or a collection thereof) can be provided (e.g., in communication with the processing arrangement 2205). The computer-accessible medium 2215 can contain executable instructions 2220 thereon. In addition or alternatively, a storage arrangement 2225 can be provided separately from the computer-accessible medium 2215, which can provide the instructions to the processing arrangement 2205 so as to configure the processing arrangement to execute certain exemplary procedures, processes, and methods, as described herein above, for example.

Further, the exemplary processing arrangement 2205 can be provided with or include an input/output ports 2235, which can include, for example a wired network, a wireless network, the internet, an intranet, a data collection probe, a sensor, etc. As shown in FIG. 22, the exemplary processing arrangement 2205 can be in communication with an exemplary display arrangement 2230, which, according to certain exemplary embodiments of the present disclosure, can be a touch-screen configured for inputting information to the processing arrangement in addition to outputting information from the processing arrangement, for example, Further, the exemplary display arrangement 2230 and/or a storage arrangement 2225 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information, It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are here incorporated by reference in their entireties.
1. Abi-Dargham A & Horga G (2016) The search for imaging biomarkers in psychiatric disorders. Nat Med 22(11):1248-1255.
2. Zecca L., et al. (2008) New melanic pigments in the human brain that accumulate in aging and block environmental toxic metals. Proc Natl Acad Sci USA 105(45):17567-17572.
3. Zucca F A, et al. (2014) Neuromelanin of the human substantia nigra: an update Neurotox Res 25(1): 13-23.
4. Zucca F A, et al. (2018) Neuromelanin organelles are specialized autolysosomes that accumulate undegraded proteins and lipids in aging human brain and are likely involved in Parkinson's disease. NPJ Parkinsons Dis 4:17.
5. Zhang, W, et al. (2011) Neuromelanin activates microglia and induces degeneration of dopaminergic neurons: implications for progression of Parkinson's disease. Neurotox Res 19(1):63-72.
6. Zucca F A, et al. (2017) Interactions of iron, dopamine and neuromelanin pathways in brain aging and Parkinson's disease. Prog. Neurobiol 155:96-119.
7. Zecca L, et al. (2004) The role of iron and copper molecules in the neuronal vulnerability of locus coeruleus and substantia nigra during aging. Proc Natl Acad Sci USA 101(26):9843-9848.
8. Sasaki M, et al. (2006) Neuroramelanin magnetic resonance imaging of locus ceruleus and substantia nigra in Parkinson's disease. Neutroreport 17(11):1215-1218.
9. Trujillo P, et al. (2017) Contrast mechanisms associated with neuromelanin-MRI. Magn Reson Med 78(5):1790-1800.
10. Sulzer D, et a (2018) Neuromelanin detection by magnetic resonance imaging (MRI) and its promise as a biomarker for Parkinson's disease. NPJ Parkinsons Dis 4:11.
11. Tosk J M, et al. (1992) Effects of the interaction between ferric iron and L-dopa melanin on T1 and T2 relaxation times determined by magnetic resonance imaging. Magn Reson Med 26(1):40-45.
12. Kuya K, et at (2016) Correlation between neuromelanin-sensitive MR imagine and (123)I-FP-CIT SPECT in patients with parkinsonism. Neuroradioiogy 58(4):351-356.
13. Fabbri M, et al. (2017) Substantia Nigra Neuromelanin as an imaging Biomarker of Disease Progression in Parkinson's Disease. J Parkinsons Dis 7(3):491-501.
14. Hatano T, el al. (2017) Neuromelanin MRI is useful for monitoring motor complications in Parkinson's and PARK2 disease, Journal of neural transmission (Vienna, Austria: 1996) 124(4):407-415.
15. Matsuura K, et at. (2013) Neuromelanin magnetic resonance imaging in Parkinson's disease and multiple system atrophy. Eur Neural 70(1-2):70-77.
16. Hirsch E. Graybiel A M, & Agid Y A (1988) Metallized dopaminergic neurons are differentially susceptible to degeneration in Parkinson's disease. Nature 334(6180):345-348.
17. Zecca L, et al. (2002) The absolute concentration of nigral neuromelanin, assayed by a new sensitive method, increases throe lout the life and is dramatically decreased in Parkinson's disease. FEBS Lett 510(3):216-220.
18. Cebrian C, et at (2014) MHC-1 expression renders catecholaminergic neurons susceptible to T-cell-mediated degeneration. Nat Commun 5:3633.
19. Sulzer D. et al. (2000) Neuromelanin biosynthesis is driven byexcess cytosolic catecholamines not accumulated by synaptic vesicles. Proc Natl Acad Sci USA 97(22):11869-11874.
20. Shihata E, et al. (2008) Use of neuromelanin sensitive MRI to distinguish schizophrenic and depressive patients and healthy individuals based on signal alterations in the substantia nigra and locus ceruleus. Biol Psychiatry 64(5):401-406.
21. Watanabe Y, et al. (2014) Neuromelanin magnetic resonance imaging reveals increased dopaminergic neuron activity in the substantia nigra of patients with schizophrenia, PLoS One 9(8):e104619.
22. Joel D & Weiner I (2000) The connections of the dopaminergic system with the striatum in rats and primates: an analysis with respect to the functional and compartmental organization of the striatum. Neuroscience 96(3):451-474.
23. Weinstein J J, et al. (2017) Pathway-Specific Dopamine Abnormalities in Schizophrenia. Biol Psychiatry 81(1):31-42.
24. Rice M W, Roberts R C, Melendez-Ferro M. & Perez-Costas F (2016) Mapping dopaminergic deficiencies in the substantia nigra/ventral tegmental area i.n. schizophrenia. Brain Struct Funct 221(1):185-201.
25. Chung C Y, et al. (2005) Cell type-specific gene expression of midbrain dopaminergic neurons reveals molecules involved in their vulnerability and protection. Hum Mol Genet 14(13):1709-1725.
26. Verney C, Zocevic N, & Puelles L (2001) Structure of longitudinal brain zones that provide the origin for the substantia nigra and ventral tegmental area in human embryos, as revealed by cytoarchitecture and tyrosine hydroxylase, calretinin, calbindin and GABA immunoreactions. J Comp Neural 429(1):22-44.
27. Damier P. Hirsch E C, Agid Y, & Graybiel A M (1999) The substantia nigra of the human brain. II. Patterns of loss of dopamine-containing neurons in Parkinson's disease. Brain 122 (Pt 8):1437-1448.
28. Fearnley M & Lees A J (1991) Ageing and Parkinson's disease: substantia nigra regional selectivity. Brain 114 (Pt 5):2283-2301.
29. Bogerts B, Hantsch J, & Herzer M (1983) A morphometric study of the dopamine-containing cell groups in the mesencephalon of normals, Parkinson patients, and schizophrenics. Biol Psychiatry 18(9):951-969.
30. Langley J, Huddleston D E, Liu C J, & Hu X (2017) Reproducibility of locus coeruleus and substantia nigra imaging with neuromelanin sensitive MRI. MAGMA 30(2):121-125.
31. Sulzer n (2011) How addictive drugs disrupt presynaptic dopamine neurotransmission. Neuron 69(4):628-649.
32. Sulzer D, Cragg S J, & Rice M E (2016) Striatal dopamine neurotransmission: regulation of release and uptake. Basal Ganglia 6(3):123 -148.
33. Kegeles L S. et al. (2010) Increased synaptic dopamine function in associative regions of the striatum in schizophrenia. Arch Gen Psychiatry 67(3):231-239.
34. Cha Y H, et al. (2013) Regional correlation between resting state FDG PET and pCASL perfusion MRI. J Cereb Blood Flow Metab 33(12):1909-1914.
35. Chen Y, et al. (2011) Voxel-level comparison of arterial spin-labeled perfusion MRI and FDG-PET in Alzheimer disease. Neurology 77(22): 1977-1985.
36. Detre. J A, Rao H. Wang D J, Chen Y F, & Wang Z (2012) Applications of arterial spin labeled MRI in the brain. J Magn Resan Imaging 35(5):1026-1037.
37. Schridde U, et al. (2008) Negative BOLD with large increases in neuronal activity. Cereb Cortex 18(8):1814-1827.
38. Hermes M, cit al. (2009) Latent state-trait structure of cerebral blood flow in resting state. Biol Psychol 80(2):196-202.
39. Egerton A, et al. (2013) Presynaptic striatal dopamine dysfunction in people at ultra-high risk for psychosis: findings in a second cohort. Biol Psychiatry 74(2):106-112.
40. Howes O D, et al. (201 1) Dopamine synthesis capacity before onset of psychosis: a prospective [18F]-DOPA PET imaging study. Am J Psychiatry 168(12):1311-1317.
41. Jauhar S, et al. (2017) A Test of the Transdiagnostic Dopamine Hypothesis of Psychosis Using Positron Emission Tomographic Imaging in Bipolar Affective Disorder and Schizophrenia. JAMA Psychiarry 74(12):1206-1213.
42. Endres C J, el al. (1997) Kinetic modeling of [11C]raclopride: combined PET-microdialysis studies. J Cereb Blood Flow Metab 17(9):932-942.
43. Laruelle M, et al. (1997) Microdialysis and SPECT measurements of amphetamine-induced dopamine release in nonhuman primates. Synapse 25(1):1-14.
44. Kay S R, Fiszhein A, & Opler L A (1987) The positive and negative syndrome scale (PANSS) for schizophrenia. Schizophr Bull 13(2):261-276.
45. Kay S R, Opler L A, & Lindenmayer J P (1988) Reliability and validity of the positive and negative syndrome scale for schizophrenics. Psychiatry Res 23(1):99-110.
46. Kitao S, et al. (2013) Correlation between pathology and neuromelanin MR imaging in Parkinson's disease and dementia with Lowy bodies. Neuroradiology 55(8):947-953.
47. Lee H, Back S Y, Chun S Y, Lee J H, & Cho H (2017) Specific visualization of neuromelaniniron complex and ferric iron in the human post-mortem substantia nigra using MR relaxometry at 7T. Neurimage.
48. Ito H. et al. (2017) Normative data of dopaminergic neurotransmission functions in substantia nigra measured with MRI and PET: Neuromelanin, dopamine synthesis, dopamine transporters, and dopamine D2 receptors, Neuroimage 158:12-17.
49. Brown C A, et al. (2013) Validation of midbrain positron emission tomography measures for nigrostriatal neurons in macaques. Ann Neurol 74(4):602-610.
50. Howes O D, et al. (2013) Midbrain dopamine function in schizophrenia and depression: a post-mortem and positron emission tomographic imaging study. Brain 136(Pt 11):3242-3251.
51. Mueller H T, Haroutunian V, Davis K L, & Meador-Woodruff J H (2004) Expression of the ionotropic glutamate receptor subunits and NMDA receptor-associated intracellular proteins in the substantia nigra schizophrenia. Brain Res Mol Brain Res 121(1-2):60-69.
52. Perez-Costas E, Melendez-Ferro M. Rice M W, Conley R R, Roberts R C (2012) Dopamine pathology in schizophrenia: analysis of total and phosphorylated tyrosine hydroxylase in the substantia nigra. Front Psychiatry 3:31.
53. Laruelle M. Abi-Dargham A, Gil R, Kegeles L, & Innis R (1999) Increased dopamine transmission in schizophrenia: relationship to illness phases. Biol Psychiatry 46(1):56-72.
54. Sasaki M, et al. (2010) Visual discrimination among patients with depression and schizophrenia and healthy individuals using semiquantitative color-coded fast spin-echo T1-weighted magnetic resonance imaging. Neuroradiology 52(2):83 -89.
55. Yamashita F. al. (2016) Detection of changes in the ventral tegmental area of patients with schizophrenia using neuromelanin-sensitive MRI. Neuroreport 27(5):289-294.
56. Kim F. et al. (2017) Presynaptic Dopanline Capacity in Patients with Treatment-Resistant Schizophrenia Taking Clozapine: An [(18)F]DOPA PET Study. Neuropsychopharmacology 42(4):941-950.
57. Chen S. Seeman P. & Liu F (2011) Antipsychotic drug binding in the substantia nigra: an examination of high metoclopramide binding in the brains of normal, Alzheimer's disease, Huntington's disease, and Multiple Sclerosis patients, and its relation to tardive dyskinesia. Synapse 65(2):119-124.
58. McGlashan T H, et al. (2006) Randomized, double-blind trial of olanzapine versus placebo in patients prodromally symptomatic for psychosis. Am J Psychiatry 163(5):790-799.
59. McGorry P D, et al. (2002) Randomized controlled trial of interventions designed to reduce the risk of progression to first episode psychosis in a clinical sample with subthreshold symptoms. Arch Gen Psychiatry 59(10):921-928
60. Cannon T D, et al. (2016) An individualized Risk Calculator for Research in Prodromal Psychosis. Am J Psychiatry 173(10):980-988.
61. Wakamatsu K, et al (20.15) Norepinephrine and its metabolites are involved in the synthesis of neuromelan in derived from the locus coeruleus. J Neurochem 135(4):768-776.
62. Clowett D V, Huang R, Velasco R, Lee T H, & Mather M (2018) Locus Coeruleus Activity Strengthens Prioritized Memories Under Arousal. J Neurosci 38(6):1558-1574.
63. Mather M, et al. (2017) Higher locus coeruleus MRI contrast is associated with lower parasympathetic influence over heart rate variability. Neuroimage 150:329-335.
64. Weinshenker D (2018) Long Road to Ruin: Noradrenergic, Dysfunction in Neurodegenerative Disease. Trends Neurosei.
65. Clewett D V, et al. (2016) Neuromelanin marks the spot: identifying a locus coeruleus biomarker of cognitive reserve in healthy aging. Neurobiol Aging 37:117-126.
66. Hammerer D, et al. (2018) Locus coeruleus integrity in old age is selectively related to memories linked with salient negative events. Proc Natl Acad Sci USA 115(9):2228-2233.
67. Chen X. et al. (2014) Simultaneous imaging of locus coeruleus and substantia nigra with a quantitative neuromelanin MRI approach. Magn Reson Imaging 32(10):1301-1306.
68. Ashbumer J (2007) A fast diffeomorphic image registration algorithm. Neuroimage 38(1):95-113.
69. Weinstein J J, et al. (2017) PET imaging of dopamine-D2 receptor internalization in schizophrenia. Mol Psychiatry.
70. Martinez D, et al. (2003) Imaging human mesolimbic dopamine transmission with positron emission tomography. Part II: amphetamine-induced dopamine release in the functional subdivisions of the striatum, J Cereb Blood Flow Metab 23 (3):285-300.
71. Lammertsma A A & Hume S P (1996) Simplified reference tissue model for PET receptor studies. Neuroimage 4(3 Pt 1):153-158.
72. Mawlawi O, et al. (2001) Imaging human mesolimbic dopamine transmission with positron emission tomography: I. Accuracy and precision of D(2) receptor parameter measurements in ventral striatum. J Cereb Blood Flow Metab 21(9):1034-1057.
73. Guo N, et al. (2010) impact of D2 receptor internalization on binding affinity of neuroimaging radiotracers. Neuropharmacology 35(3):806-817.
74. Ito K, Haga T, Lameh J, & Sadee W (1999) Sequestration of dopamine D2 receptors depends on coexpression of G-protein-coupled receptor kinases 2 or 5. Eur J Biochem 260(1):112-119.
75. Skinbierg M, et at (2010) D2 dopamine receptor internalization prolongs the decrease of radioligand binding after amphetamine: a PET study in a receptor internalization-deficient mouse model. Neuroimage 50(4):1402-1407.
76. Wong A M, Yan F X, & Liu H L (2014) Comparison of three-dimensional pseudo-continuous arterial spin labeling perfusion imaging with gradient-echo and spin-echo dynamic susceptibility contrast MRI. J Magn Reran Imaging 39(2):427-433.
77. Weinstein J J, et al. (2017) PET imaging of dopamine-D2 receptor internalization in schizophrenia. Mol Psychiatry.
78. Cassidy C M, et al. (2018) A Perceptual Inference Mechanism for Hallucinations Linked to Striatal Dopamine, Curr Biol 28(41:503-514 e504.
79. Skinbjerg M, et al. (2010) D2 dopamine receptor internalization prolongs the decrease of radioligand binding after amphetamine: a PET study in a receptor internalization-deficient mouse model. Neuroimage 50(4):1402-1407.
80. Guo N. et al, (2010) impact of D2 receptor internalization on binding affinity of neuroimaging radiotracers. Neuropsychopharmacology 35(3):806-817.
81. Ito K, Haga T, Lameh J, & Sadee W (1999) Sequestration of dopamine D2 receptors depends on coexpression of G-protein-coupled receptor kinases 2 or 5. Eur J Biochem 260(1):112-119.
82. Wong A M, Yan F X, & Liu H L (2014) Comparison of three-dimensional pseudo-continuous arterial spin labeling perfusion imaging with gradient-echo and spin-echo dynamic susceptibility contrast MRI. J Magn Reson Imaging 39(21:427-433.
83. First M, Spitzer R, Gibbon M, & (1995) Structured Clinical interview for DSM-IV Axis I Disorders (SCID-T/P, Version 2.0) (Biometrics Research Dept., New York State Psychiatric Institute, New York).
84. Numberger J I, Jr., et al. (1994) Diagnostic interview for genetic studies. Rationale, unique features, and training. NIMH Genetics Initiative. Arch Gen Psychiatry 51(11):849-859; discussion 863-844.
85. Kay S R, Fiszbein A, & Opler L A (1987) The positive and negative syndrome scale (PANSS) for schizophrenia. Schizophr Bull 13(2):261-276.
86. Miller T J, et al. (2003) Prodromal assessment with the structured interview for prodromal syndromes and the scale of prodromal symptoms: predictive validity, interrater reliability, and training to reliability. Schizophr Bull 29(4):703-715.
87. Hollingshead A B (1975) Four factor index of social status. (Working paper published by the author., New Haven, Conn.).
88. Shrout P E & Fleiss J L (1979) intraclass correlations: uses in assessing rater reliability. Psychol Bull 86(2):420-428.
89. Cicchetti D V (1994) Guidelines, criteria, and rules of thumb for evaluating normed and standardized assessment instruments in psychology. Psychological Assessment.6(4):pp.
90. Abi-Dargham, A., Rodenhiser, Printz, D., Zea-Ponce, Y., Gil, R., Kegcles, L, S., Weiss, R., Cooper, T. B., Mann, J. J., Van Heertum, Gorman, M., Laruelle, M., 2000. Increased baseline occupancy of D-2 receptors by dopamine in schizophrenia. Proceedings of the National Academy of Sciences of the United States of America 97, 8104-8109.
91. Aiha, Y., Sakakibara, R., Tsuyusaki, Y Oki, T., Ogata, T., Tateno, F., Terada H., Neuromelanin-sensitive MRI in the differential diagnosis of elderly white a matter lesion from Parkinson's disease. Neurology and Clinical Neuroscience.
92. Andersson, J. L., Jenkinson, M., Smith, S., 2007. Non-linear registration aka Spatial normalisation FMRIB Technial Report TR07JA2, FMRIB Analysis Group of the University of Oxford.
93. Ashburner, J., 2007. A fast diffeomorphic image registration algorithm. NeuroImage 38, 95-113.
94. Ashbumer, Friston, 2005. Unified segmentation. NeuroImage 26, 839-851.
95. Avants, B. B., Epstein, C. L., Grossman, M., Gee, J. C., 2008. Symmetric diffeoniorphic image registration with cross-correlation: evaluating automated labeling of elderly and neurodegenerative brain. Medical image analysis 12, 26-41.
96. Avants, B. B., Tustison, N., Song, G., 2009. Advanced normalization tools (ANTS). Insight j 2, 1-35.
97. Cassidy, C. M., Zucca, F. A., Girgis, R. R., Baker, S. C., Weinstein, J. J., Sharp, M. E., Bellei, C., Valmadre, A., Vanegas, N., Kegeles, L. S., Brucato, G., Jung Kang, U., Sulzer, D., Zecea, L. Abi-Dargham, A., Horga, G., 2019. Neuromelanin-sensitive MRI as a noninvasive proxy measure of dopamine function in the human brain. Proceedings of the National Academy of Sciences 116, 5108-5117.
98. Cebrián, C., Zucca. F. A., Mauri, P., Steinbeck, J. A., Studer, L., Schemer, C. R., Kanter, E., Budhu, S. Mandelbaum, J., Vonsattel, J. P., 2014. MHC-I expression renders catecholatninergic neurons susceptible to T-cell-mediated degeneration. Nature communications 5, 3633.
99. Chen, X., Huddleston, D. E., Langley, J., Ahn, S., Barnum, Factor, S. A., Levey, A. I., Hu, X., 2014. Simultaneous imaging of locus coeruleus and substantia nigra with a quantitative neuromelanin MRI approach. Magnetic resonance imaging 32, 1301-1306.
100. Cicchetti, D. V., 1994. Guidelines, criteria, and rules of thumb for evaluating normed and standardized assessment instruments in psychology. Psychological assessment 6, 284.
101. Cowen, D., 1986. The melanoneurons of the human cerebellum (nucleus pigmentosus cerebellaris) and homologues in the monkey. Journal of Neuropathology & Experiniental Neurology 45, 205-221.
102. Danner, P., Hirsch, E., Agid, Y., Graybiel. A., 1999. The substantia nigra of the human brain: II. Patterns of loss of dopamine-containing neurons in Parkinson's disease. Brain 122, 1437-1448.
103. Davis, K. L., Kahn. R. S., Ko, G., Davidson, M., 1991. Dopamine in schizophrenia—a review and reconceptualization. American Journal of Psychiatry 148, 1474-1486.
104. Depue, R. A., Collins, P. F., 1999. Neurobiology of the structure of personality: Dopamine, facilitation of incentive motivation, and extraversion. Behavioral and brain sciences 22, 491-517.
105. Dunlop, B. W., Nemerotf, C. B., 2007. The role of dopamine in the pathophysiology of depression. Archives of general psychiatry 64. 327-337.
106. Fahn, S., Sulzer, D., 2004. Neurodegeneration and neuroprotection in Parkinson disease. NeuroRx 1, 139-154.
107. Fearnley, J. M., Lees, A. J., 1991. Ageing and Parkinson's disease: substantia nigra regional selectivity. Brain 114, 2283-2301.
108. Fields, H. L., Hjelmstad, G. O., Margolis, E. B., S. M., 2007. Ventral tegmental area neurons in learned appetitive behavior and positive reinforcement. Annu. Rev. Neurosci. 30. 289-316.
109. Griswold, M. A., Jakob, P. M., Heidemann, R. M., Nittka, M., Jellus, V., Wang, J., Kiefer, B. Haase, A., 2002. Generalized autocalibrating partially parallel acquisitions (GRAPPA). Magnetic resonance in medicine 47, 1202-1210.
110. Haber, S., Ryoo, H., Cox, C., Lu, W., 1995. Subsets of midbrain dopaminergic neurons in monkeys are distinguished by different levels of mRNA for the dopamine transporter: comparison with the mRNA for the D2 receptor, tyrosine hydroxylase and calbindin immunoreactivity. Journal of Comparative Neurology 362, 400-410.
111. Hatano, T., Okuzumi, A., Kamagata, K., Daida, K., Taniguchi, D., Hori, M., Yoshino, H., Aoki, S., Hattori, N., 2017, Neuromelanin MRI is useful for monitoring motor complications in Parkinson's and PARK2 disease, Journal of Neural Transmission 124, 407-415.
112. Hirsch, F., Graybiel, A. M., Agid, Y. A., 1988. Metallized dopaminergic neurons are differentially susceptible to degeneration in Parkinson's disease. Nature 334, 345.
113. Huddleston, D. E., Langley, J., Sedlacik, J., Boelmans, K., Factor, S. A., Hu, X. P., 2017. In vivo detection of lateral—ventral tier nigral degeneration in Parkinson's disease. Human brain mapping 38, 2627-2634.
114. Isaias, I.U., Trujillo, P., Summers, P., Marotta, G., Mainardi, L., Pezzoli, G., Zecca, L., Costa, A., 2016. Neuromelanin imaging and dopaminergic loss in Parkinson's disease. Frontiers in aging neuroscience 8, 196.
115. Jenkinson, M., Beckmann, C. F., Behrens, T. E., Woolrich M. W., Smith, S. M., 2012. FSL, NeuroImage 62, 782-790.
116. Kelley, A. E., Berridge, K. C., 2002. The neuroscience of natural rewards: relevance to addictive drugs. Journal of neuroscience 22. 3306-3311.
117. Kitao, S., Matsusue, E., Fujii, S., Miyoshi, F., Kaminou, T., Kato, S., Ito, H., Ogawa, T., 2013. Correlation between pathology and neuromelanin MR imaging in Parkinson's disease and dementia with Lewy bodies. Neuroradiology 55, 947-953.
118. Klein, A., Andersson, J., Ardekani, B. A., Ashbumer, J., Avants, B., Chiang, M. C., Christensen, G. E., Collins D. L., Gee, J., Hellier, P., Song, J. H., Jenkinson, M., Lepage, C. Rueckert, D., Thompson, P., Vercauteren, T., Woods, R P., Mann J. J., Parsey R. V., 2009. Evaluation of 14 nonlinear deformation algorithms applied to human brain MRI registration, NeuroImage. 46, 786-802.
119. Koob, G. E., Sarum, P. P., Bloom, F. E., 1998. Neuroscience of addiction, Neuron 21, 467-476.
120. Langley, J., Huddleston, D. E., Chen. X., Sedlacik, J., Zachariab, N., Hu, X., 2015. A multicontrast approach for comprehensive imaging of substantia nigra. NeuroImage 112, 7-13.
121. Langley, J., Huddleston, D. E., Liu, C. J., Hu, X., 2017. Reproducibility of locus coeruleus and substantia nigra imaging with neuromelanin sensitive MRI. Magnetic Resonance Materials in Physics, Biology and Medicine 30, 121-125.
122. Laruelle, M., Abi-Dargham, A., vanDyck, Gil, R., Dsouza, C. D., Erdos, J., McCance, E., Rosenblatt, W., Fingado, C., Zoghbi, S. S., Baldwin, R. M., Seibyl, J. P., Krystal, J. H., Citarney, D. S., Innis, R. B., 1996. Single photon emission computerized tomography imaging of amphetamine-induced dopamine release in drug-free schizophrenic subjects. Proceedings of the National Academy of Sciences of the United States of America 93, 9235-9240.
123. Liang, C. L., Nelson, O., Yazdani, Pasbakhsh, P., German, D. C., 2004. Inverse relationship between the contents of neuromelanin pigment and the vesicular monoamine transporter-2: human midbrain dopamine neurons. Journal of Comparative Neurology 473, 97-106.
124. Matsuura, K., Maeda, M., Yata, K., Ichiba, Y., Yamaguchi, Kanamaru, K., Tonmimoto, H., 2013. Neuromelanin magnetic resonance imaging in Parkinson's disease and multiple system atrophy. European neurology 70, 70-77.
125. Montague, P. R., Dayan, Sejnowski, T. J., 1996. A framework for mesencephalic dopamine systems based on predictive Hebbian learning. Journal of neuroscience 16, 1936-1947.
126. Nestler, E. J., Carlezon Jr. W. A., 2006. The mesolimbic dopamine reward circuit in depression. Biological psychiatry 59, 1151-1159.
127. Ohtsuka, C., Sasaki, M., Konno, K., Kato, K., Takahashi, J., Yamashita, F., Terayama, Y., 2014. Differentiation of early-stage parkinsonisms using neuromelanin-sensitive magnetic resonance imaging. Parkinsonism & related disorders 20, 755-760.
128. Ohtsuka, C., Sasaki, M., Konno, K., Koide, M., Kato, K., Takahashi, J., Takahashi, S., Kudo, K., Yamashita, F., Terayama, Y., 2013. Changes in substantia nigra and locus coeruleus in patients with early-stage Parkinson's disease using neuromelanin-sensitive MR imaging. Neuroscience letters 541, 93-98.
129. Parker, D. L., Buswell, H. R., Goodrich, K. C., Alexander, A L., Keck, N., Tsuruda, J. S., 1995. The application of magnetization transfer to MR angiography with reduced total power. Magnetic resonance in medicine 34, 283-286.
130. Pauli, W. M. Nili, A. N., Tyszka, J. M., 2018. A high-resolution probabilistic in vivo atlas of human subcortical brain nuclei. Scientific data 5, 180063.
131. Penny, W. D., Friston, K. J., Ashburner, Kiebel, S. J., Nichols, T. E., 2011. Statistical parametric mapping: the analysis of functional brain images. Elsevier.
132. Robson, P. M., Grant, A. K., Madhuranthakam, A. J., Lattanzi, R., Sodickson, D. K., McKenzie. C. A., 2008. Comprehensive quantification of signal-to-noise ratio and g-factor tier image-based and k-space-based parallel imaging reconstructions. Magnetic resonance in medicine 60, 895-907.
133. Roeper, J., 2013. Dissecting the diversity of midbrain dopamine neurons. Trends in neurosciences 36, 336-342.
134. Sasaki, M., Shibata, E., Kudo, K., Tohyama K., 2008. Neuromelanin-sensitive MRI. Clinical Neuromdiology 18, 147-153.
135. Sasaki, M., Shibata, E., Tohyama, K., Takahashi. J., Otsuka, K., Tsuchiya K., Takahashi, S., Ehara, S., Terayama, Y., Sakai, A., 2006. Neuromelanin magnetic resonance imaging of locus ceruleus and substantia nigra in Parkinson's disease. Neuroreport 17, 1215-1218.
136. Schultz, W., 1998. Predictive regard signal of dopamine neurons. Journal of neurophysiology 80, 1-27.
137. Schultz, W., 2007, Behavioral dopamine signals. Trends in neurosciences 30, 203-210.
138. Schwarz, S., Bajaj, N., Morgan, P., Reid, S., Gowland, P., Auer, D., 2013. Magnetisation transfer contrast to enhance detection of neuromelanin loss at 3T in Parkinson's disease. Proc. Int. Soc. Mag. Reson. Med, p. 2848.
139. Schwarz, S. T., Rittman, T., Gontu, V., Morgan, P. S., Bajaj, N., Auer, D. P., 2011. T1-weighted MRI shows stage-dependent substantia nigra signal loss in Parkinson's disease. Movement Disorders 26, 1633-1638.
140. Shibata, E., Sasaki, M., Tohyania, K., Otsuka, K., Endob, J., Terayama, Y., Sakai, A., 2008. Use of neuromelanin-sensitive MRI to distinguish schizophrenic and depressive patients and healthy individuals based on signal alterations in the substantia nigra and locus ceruleus. Biological pychiatiy 64, 401-406.
141. Shrout, P. E., Fleiss, J. L., 1979. Intraclass correlations: uses in assessing rater reliability. Psychological bulletin 86, 420.
142. Sulzer, D., Bogulaysky, J., Larsen, K. E., Behr, G., Karatekin, E., Kleinman, M. H., Turro, N., Krantz, D., Edwards, R. H., Greene, L. A., 2000. Neuromelanin biosynthesis is driven by excess cytosolic catecholamines not accumulated by synaptic vesicles. Proceedings of the National Academy of Sciences 97, 11869-11874.
143. Sulzer, D., Cassidy, C., Horga, G., Kang., U. J., Faint, S., Castilla, L., Pezzoli, G., Langley, J., Hu, X. P., Zucca, F. A., 2018. Neuromelanin detection by magnetic resonance imaging (MRI) and its promise as a biomarker for Parkinson's disease, NRJ Parkinson's disease 4, 11.
144. Sulzer, D., Surmeier, D. J., 2013. Neuronal vulnerability, pathogenesis, and Parkinson's disease. Movement Disorders 28, 715-724.
145. Sulzer, D., Zeeca, L., 1999. Intraneuronal dopamine-quinone synthesis: a review. Neurotoxicity research 1, 181-195.
146. Trujillo, P., Summers, P. E., Ferrari, E., Zucca, F. A., Sturini, M., Mainardi, L. T., Cerutti, S., Smith A. K., Smith, S. A., Zecca, L., 2017. Contrast mechanisms associated with neuromelanin-MRI. Magnetic resonance in medicine 78, 1790-1800.
147. Ungless, M. A., Magill, P J., Bolam, J. P., 2004. Uniform inhibition of dopamine neurons in the ventral tegmental area by aversive stimuli. Science 303, 2040-2042.
148. Wang, J., Huang, Z., Li, Y., Ye, F., Wang, C., Zhang, Y., Cheng, X., Fei, G., Liu, K., Zeng, M. 2019. Neuromelanin-sensitive MRI of the substantia nigra: an imaging biomarker to differentiate essential tremor from tremor-dominant Parkinson's disease. Parkinsonism & related disorders 58, 3-8.
149. Wang, J., Li. Y., Huang, Z., Wan, W., Zhang, Y., Wang, C., Cheng, X., Ye, F., Liu, K., Fei, G., 2018. Neuromelanin-sensitive magnetic resonance imaging features of the substantia nigra and locus coeruleus in de novo Parkinson's disease and its phenotypes. European journal of neurology 25, 949-e973.
150. Watanabe, Y., Tanaka, H., Tsukabe, A., Kunitomi, Y., Nishizawa, M., Hashimoto, R., Yamamori, H., Fujimoto, M., Fukunaga, M., Tomiyama, N., 2014. Neuromelanin magnetic resonance imaging reveals increased dopaminergic neuron activity in the substantia nigra of patients with schizophrenia. PLoS One 9, e104619.
151. Weinstein, J. J., Chohan, M. O., Slifstein, M., Kegeles, L. S., Moore, H., Abi-Dargham, A., 2017. Pathway-specific dopamine abnormalities in schizophrenia. Biological psychiatry 81, 31-42.
152. Xing, Y., Sapuan, A., Dineen, R. A., Auer, D. P., 2018. Life span pigmentation changes of the substantia nigra detected by neuromelanin-sensitive MRI. Movement. Disorders 33, 1792-1799.
153. Yamashita, F., Sasaki, M., Fukumoto, K., Otsuka, K., Uwano, I., Kameda, H., Endoh, J., Sakai, A., 2016. Detection of changes in the ventral tegmental area of patients with schizophrenia using neuromelanin-sensitive MRI. Neuroreport 27, 289-294.
154. Zecca, L., Beilei, C., Costi, P., Albertini, A., Monzani, E., Casella, L., Gatiorini, M., Bergamaschi, L., Moscatelli, A., Turro N. J., 2008. New melanic pigments in the human brain that accumulate in aging and block environmental toxic metals. Proceedings of the National Academy of Sciences 105, 17567-17572.
155. Zecca, L., Costi, P., Mecacci, C., Ito, S., Terreni, M., Sonnino, S., 2000. Interaction of human substantia nigra neuromelanin with lipids and peptides. Journal of neurochemistry 74, 1758-1765.
156. Zecca, L., Shima, T., Stroppolo, A., Goj, C., Battiston G., Gerbasi, Sarna, T., Swartz, H., 1996. Interaction of neuromelanin and iron in substantia nigra and other areas of human brain. Neuroscience 73, 407-415.
157. Zecca, Stroppolo, A., Gatti, A., Tampe D., Toscani, M., Gallorini, M., Glaveri, G., Arosio, P., Santambrogio, P., Fariello, R. G., 2004. The role of iron and copper molecules in the neuronal vulnerability of locus coeruleus and substantia nigra during aging. Proceedings of the National Academy of Sciences 101, 9843-9848.
158. Zucca, F. A., Basso, F., Cupaioli, F. A., Ferrari, E., Sulzer, D., Casella, L., Zeeca, L., 2014. Neuromelanin of the human substantia nigra: an update, Neurotoxicity research 25, 13-23.
159. Zucca, F. A., Vanna, R., Cupaioli, F. A., Belle C., De Palma, A., Di Silvestro, D., Maud, P., Grassi, S., Prinetti, A., Casella L., 2018. Neuromelanin organelles are specialized autolysosomes that accumulate undegraded proteins and lipids in aging human brain and are likely involved in Parkinson's disease. NPJ Parkinson's disease 4, 17.

1. A non-transitory computer-accessible medium having stored thereon computer-executable instructions for determining a dopamine function of at least one patient, wherein, when a computer arrangement executes the instructions, the computer arrangement is configured to perform procedures comprising:
receiving imaging information of a brain of the at least one patient;
determining a Neuromelanin (NM) concentration of the at least one patient based on the imaging information; and
determining the dopamine function based on the NM concentration.

2. The computer-accessible medium of claim 1, wherein the computer arrangement is configured to determine the NM concentration using a voxel-wise analysis procedure.

3. The computer-accessible medium of claim 2, wherein the computer arrangement is configured to use the voxel-wise analysis procedure to determine at least one topographical pattern within a substantia nigra (SN) of the brain of the at least one patient.

4. The computer-accessible medium of claim 3, wherein the at least one topographical pattern includes at least one pattern of cell loss in the SN.

5. The computer-accessible medium of claim 1, wherein the NM concentration is based on a NM loss in the brain of the at least one patient.

6. The computer-accessible medium of claim 1, wherein the imaging information is magnetic resonance imaging (MRI) information.

7. The computer-accessible medium of claim 6, wherein the computer arrangement is further configured to determine a variance in the NM concentration using a NM-MRI contrast-to-noise ratio (CNR).

8. The computer-accessible medium of claim 7, wherein the computer arrangement is further configured to determine the NM-MRI CNR at each voxel in the imaging information.

9. The computer-accessible medium of claim 8, wherein the computer arrangement is configured to determine the NM-MRI CNR as a relative change in a NM-MRI signal intensity from a reference region of white matter tracts in the brain of the at least one patient.

10. The computer-accessible medium of claim 1, wherein the computer arrangement is further configured to determine information correlating with a brain disorder of the at least one patient based on the dopamine function.

11. The computer-accessible medium of claim 10, wherein the brain disorder includes at least one of (i) schizophrenia, (ii) bipolar disorder, or (iii) Parkinson's disease.

12. The computer-accessible medium of claim 10, wherein the computer arrangement is further configured to determine further information correlating with a severity of the brain disorder based on the dopamine function.

13. The computer-accessible medium of claim 1, wherein the imaging information includes (i) at least one sagittal three-dimensional (3D) T1w image, (ii) at least one coronal 3D T1w image, and (iii) at least one axial 3D T1w image.

14. The computer-accessible medium of claim 1, wherein the computer arrangement is further configured to determine a magnetic resonance imaging (MRI) volume placement in the imaging information.

15. The computer-accessible medium of claim 14, wherein the computer arrangement is configured to determine the MRI volume placement by:
a) identifying a sagittal image showing a largest separation between a midbrain of the at least one patient and a thalamus of the at least one patient; and
b) determining a coronal image that has a coronal plane in the sagittal image that identifies a most anterior aspect of the midbrain.

16. The computer-accessible medium of claim 15, wherein the computer arrangement is configured to determine the MRI volume placement by:
a) determining an axial plane in the coronal image that identifies an inferior aspect of a third ventricle of the brain of the at least one patient; and
b) setting a superior boundary of the NM-MRI volume to be within a particular distance superior to the axial plane.

17. The computer-accessible medium of claim 16, wherein the particular distance is about 3 mm.

18. A method for determining a dopamine function of at least one patient, comprising:
receiving imaging information of a brain of the at least one patient;
determining a Neuromelanin (NM) concentration of the at least one patient based on the imaging information; and
determining the dopamine function based on the NM concentration.

19-31. (canceled)

32. The method of claim 31, wherein the MM volume placement is determined by:
a) identifying a sagittal image showing a largest separation between a midbrain of the at least one patient and a thalamus of the at least one patient; and
b) determining a coronal image that has a coronal plane in the sagittal image that identifies a most anterior aspect of the midbrain.

33-34. (canceled)

35. A system for determining a dopamine function of at least one patient, comprising:
a computer hardware arrangement configured to:
receive imaging information of a brain of the at least one patient;
determine a Neuromelanin (NM) concentration of the at least one patient based on the imaging information; and
determine the dopamine function based on the NM concentration.

36-51. (canceled)
